SEQUENCE LISTING (84 sequences)

SEQ ID NO: 1 - protein, 7 aa, Artificial Sequence
  LCDR1
SVSSAVA

SEQ ID NO: 2 - protein, 7 aa, Artificial Sequence
  LCDR2
SASSLYS

SEQ ID NO: 3 - protein, 6 aa, Artificial Sequence
  LCDR3
QHYSLF

SEQ ID NO: 4 - protein, 6 aa, Artificial Sequence
  LCDR3
QGFAPF

SEQ ID NO: 5 - protein, 6 aa, Artificial Sequence
  LCDR3
QSYGPF

SEQ ID NO: 6 - protein, 6 aa, Artificial Sequence
  LCDR3
QSYSPF

SEQ ID NO: 7 - protein, 6 aa, Artificial Sequence
  LCDR3
QHYALF

SEQ ID NO: 8 - protein, 6 aa, Artificial Sequence
  LCDR3
QHYGLI

SEQ ID NO: 9 - protein, 6 aa, Artificial Sequence
  LCDR3
QAGWPI

SEQ ID NO: 10 - protein, 6 aa, Artificial Sequence
  LCDR3
QAYSPF

SEQ ID NO: 11 - protein, 7 aa, Artificial Sequence
  LCDR3
QHYSYPI

SEQ ID NO: 12 - protein, 6 aa, Artificial Sequence
  LCDR3
QAYALI

SEQ ID NO: 13 - protein, 10 aa, Artificial Sequence
  HCDR1
FNISYSSIHW

SEQ ID NO: 14 - protein, 10 aa, Artificial Sequence
  HCDR1
FNLYYSSIHW

SEQ ID NO: 15 - protein, 10 aa, Artificial Sequence
  HCDR1
FNLYYYSMHW

SEQ ID NO: 16 - protein, 10 aa, Artificial Sequence
  HCDR1
FNIYYSYMHW

SEQ ID NO: 17 - protein, 10 aa, Artificial Sequence
  HCDR1
FNLYYYSIHW

SEQ ID NO: 18 - protein, 10 aa, Artificial Sequence
  HCDR1
FNIYYSSMHW

SEQ ID NO: 19 - protein, 11 aa, Artificial Sequence
  HCDR1
FNISSYYSIHW

SEQ ID NO: 20 - protein, 11 aa, Artificial Sequence
  HCDR2
AYIYPYYGYTY

SEQ ID NO: 21 - protein, 11 aa, Artificial Sequence
  HCDR2
ASIYSYSSYTS

SEQ ID NO: 22 - protein, 11 aa, Artificial Sequence
  HCDR2
ASIYPYSSYTS

SEQ ID NO: 23 - protein, 11 aa, Artificial Sequence
  HCDR2
ASIYSSSGYTS

SEQ ID NO: 24 - protein, 11 aa, Artificial Sequence
  HCDR2
ASIYSSSSSTY

SEQ ID NO: 25 - protein, 7 aa, Artificial Sequence
  HCDR3
RGYGIDY

SEQ ID NO: 26 - protein, 7 aa, Artificial Sequence
  HCDR3
RSYAMDY

SEQ ID NO: 27 - protein, 7 aa, Artificial Sequence
  HCDR3
RHYAMDY

SEQ ID NO: 28 - protein, 7 aa, Artificial Sequence
  HCDR3
RGYAFNY

SEQ ID NO: 29 - protein, 7 aa, Artificial Sequence
  HCDR3
RGYAMDY

SEQ ID NO: 30 - protein, 12 aa, Artificial Sequence
  HCDR3
RGYYAGYFGIDY

SEQ ID NO: 31 - protein, 7 aa, Artificial Sequence
  HCDR3
RSFAMDY

SEQ ID NO: 32 - protein, 7 aa, Artificial Sequence
  HCDR3
RGYGLDY

SEQ ID NO: 33 - protein, 234 aa, Artificial Sequence
  Light Chain- 1095
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQHYSLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 234 aa, Artificial Sequence
  Light Chain- 1096
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQGFAPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 234 aa, Artificial Sequence
  Light Chain- 1097
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQSYGPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 234 aa, Artificial Sequence
  Light Chain- 1098
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQSYSPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 234 aa, Artificial Sequence
  Light Chain- 1099
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQHYALFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 234 aa, Artificial Sequence
  Light Chain- 1100
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQHYGLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 234 aa, Artificial Sequence
  Light Chain- 1101
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQAGWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 234 aa, Artificial Sequence
  Light Chain- 1102
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQAYSPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 235 aa, Artificial Sequence
  Light Chain- 1103
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQHYSYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 234 aa, Artificial Sequence
  Light Chain- 1104
MVLQTQVFISLLLWISGAYGSDIQMTQSPSSLSASVGDRVTITCRASQSVSSAVAWYQQKPGKAPKLLIYSASSLYSGVPSRFSGSRSGTDFTLTISSLQPEDFATYYCQQAYALITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 462 aa, Artificial Sequence
  Heavy Chain- 1095
MKHLWFFLLLVAAPRWVLSEISEVQLVESGGGLVQPGGSLRLSCAASGFNISYSSIHWVRQAPGKGLEWVAYIYPYYGYTYYADSVKGRFTISADTSKNTAYLQMNSLRAEDTAVYYCARGYGIDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYQKSLSLSPGK

SEQ ID NO: 44 - protein, 463 aa, Artificial Sequence
  Heavy Chain- 1096
MKHLWFFLLLVAAPRWVLSEISEVQLVESGGGLVQPGGSLRLSCAASGFNLYYSSIHWVRQAPGKGLEWVASIYSYSSYTSYADSVKGRFTISADTSKNTAYLQMNSLRAEDTAVYYCARSYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 45 - protein, 463 aa, Artificial Sequence
  Heavy Chain- 1097
MKHLWFFLLLVAAPRWVLSEISEVQLVESGGGLVQPGGSLRLSCAASGFNLYYYSMHWVRQAPGKGLEWVASIYSYSSYTSYADSVKGRFTISADTSKNTAYLQMNSLRAEDTAVYYCARSYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 46 - protein, 463 aa, Artificial Sequence
  Heavy Chain- 1098
MKHLWFFLLLVAAPRWVLSEISEVQLVESGGGLVQPGGSLRLSCAASGFNIYYSYMHWVRQAPGKGLEWVASIYSYSSYTSYADSVKGRFTISADTSKNTAYLQMNSLRAEDTAVYYCARHYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 47 - protein, 463 aa, Artificial Sequence
  Heavy Chain- 1099
MKHLWFFLLLVAAPRWVLSEISEVQLVESGGGLVQPGGSLRLSCAASGFNLYYYSIHWVRQAPGKGLEWVASIYPYSSYTSYADSVKGRFTISADTSKNTAYLQMNSLRAEDTAVYYCARGYAFNYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 48 - protein, 463 aa, Artificial Sequence
  Heavy Chain- 1100
MKHLWFFLLLVAAPRWVLSEISEVQLVESGGGLVQPGGSLRLSCAASGFNLYYSSIHWVRQAPGKGLEWVASIYSYSSYTSYADSVKGRFTISADTSKNTAYLQMNSLRAEDTAVYYCARGYAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 49 - protein, 468 aa, Artificial Sequence
  Heavy Chain- 1101
MKHLWFFLLLVAAPRWVLSEISEVQLVESGGGLVQPGGSLRLSCAASGFNIYYSSMHWVRQAPGKGLEWVAYISSSYGYTYYADSVKGRFTISADTSKNTAYLQMNSLRAEDTAVYYCARGYYAGYFGIDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 50 - protein, 463 aa, Artificial Sequence
  Heavy Chain- 1102
MKHLWFFLLLVAAPRWVLSEISEVQLVESGGGLVQPGGSLRLSCAASGFNIYYSSMHWVRQAPGKGLEWVASIYSYSSYTSYADSVKGRFTISADTSKNTAYLQMNSLRAEDTAVYYCARSFAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 51 - protein, 463 aa, Artificial Sequence
  Heavy Chain- 1103
MKHLWFFLLLVAAPRWVLSEISEVQLVESGGGLVQPGGSLRLSCAASGFNISYYSIHWVRQAPGKGLEWVASIYSSSGYTSYADSVKGRFTISADTSKNTAYLQMNSLRAEDTAVYYCARGYGLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 52 - protein, 463 aa, Artificial Sequence
  Heavy Chain- 1104
MKHLWFFLLLVAAPRWVLSEISEVQLVESGGGLVQPGGSLRLSCAASGFNISYYSIHWVRQAPGKGLEWVASIYSSSSSTYYADSVKGRFTISADTSKNTAYLQMNSLRAEDTAVYYCARGYGLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 53 - DNA, 1400 bp, Artificial Sequence
  Heavy Chain- 1095
atgaagcacctgtggttctttctgctgctggtggccgctccaagatgggtgctgagcgagatctctgaggtgcagctggtggagagcggaggaggactggtgcagccaggaggatccctgagactgagctgcgccgcttctggcttcaacatctcttactccagcatccattgggtgcgccaggctccaggcaagggactggagtgggtggcttacatctatccttactatggctacacctactatgccgactctgtgaagggcaggttcaccatctccgccgataccagcaagaacacagcttatctgcagatgaactccctgagggccgaggacaccgccgtgtactattgtgctcggggctacggcatcgattattggggccagggcaccctggtgacagtgtcttccgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 54 - DNA, 1400 bp, Artificial Sequence
  Heavy Chain- 1096
atgaagcacctgtggttctttctgctgctggtggctgctccaagatgggtgctgtccgagatcagcgaggtgcagctggtggagagcggaggaggactggtgcagcctggaggcagcctgagactgtcttgcgccgcttccggcttcaacctgtactattccagcatccattgggtgcgccaggctccaggcaagggactggagtgggtggcttctatctactcctattcttcctacaccagctatgccgactctgtgaagggcaggttcaccatctccgccgataccagcaagaacacagcttacctgcagatgaactccctgagggccgaggacaccgccgtgtactattgtgcccggtcctacgctatggattattggggccagggcaccctggtgacagtgagctctgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 55 - DNA, 1400 bp, Artificial Sequence
  Heavy Chain- 1097
atgaagcacctgtggttctttctgctgctggtggctgctccaagatgggtgctgagcgagatctctgaggtgcagctggtggagtctggaggaggactggtgcagcctggaggatctctgagactgtcctgcgctgcctccggcttcaacctgtactattactccatgcattgggtgcgccaggctccaggcaagggactggagtgggtggcttccatctatagctactccagctatacctcttacgccgactccgtgaagggcaggttcaccatcagcgccgatacctctaagaacacagcttatctgcagatgaactccctgagggccgaggacaccgccgtgtactactgtgcccggagctatgctatggattactggggccagggcaccctggtgacagtgtcttccgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 56 - DNA, 1400 bp, Artificial Sequence
  Heavy Chain- 1098
atgaagcacctgtggttctttctgctgctggtggctgctccaagatgggtgctgagcgagatctctgaggtgcagctggtggagtccggaggaggactggtgcagcctggaggatctctgagactgtcctgcgctgcctccggcttcaacatctactatagctacatgcattgggtgcgccaggctccaggcaagggactggagtgggtggcttccatctacagctattccagctacacctcttatgccgactccgtgaagggcaggttcaccatcagcgccgatacctctaagaacacagcttatctgcagatgaactccctgagggccgaggacaccgccgtgtactattgtgcccggcactacgctatggattattggggccagggcaccctggtgacagtgtcttccgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 57 - DNA, 1400 bp, Artificial Sequence
  Heavy Chain- 1099
atgaagcacctgtggttctttctgctgctggtggccgctccaagatgggtgctgagcgagatctctgaggtgcagctggtggagtccggaggaggactggtgcagccaggaggatccctgagactgagctgcgccgcttctggcttcaacctgtactattacagcatccattgggtgcgccaggctccaggcaagggactggagtgggtggcttctatctatccttactccagctatacctcctacgccgacagcgtgaagggcagattcacaatctctgccgatacctccaagaacacagcttatctgcagatgaactccctgagggccgaggacaccgccgtgtactactgtgcccggggctatgcttttaattactggggccagggcaccctggtgacagtgtcttccgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 58 - DNA, 1400 bp, Artificial Sequence
  Heavy Chain- 1100
atgaagcacctgtggttctttctgctgctggtggctgctccaagatgggtgctgtccgagatcagcgaggtgcagctggtggagtctggaggaggactggtgcagcctggaggaagcctgagactgtcttgcgccgcttccggcttcaacctgtactattccagcatccattgggtgcgccaggctccaggcaagggactggagtgggtggcttctatctactcctattcttcctacaccagctatgccgactctgtgaagggcaggttcaccatctccgccgataccagcaagaacacagcttacctgcagatgaactccctgagggccgaggacaccgccgtgtactattgtgcccggggctacgctatggattattggggccagggcaccctggtgacagtgagctctgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 59 - DNA, 1415 bp, Artificial Sequence
  Heavy Chain- 1101
atgaagcacctgtggttctttctgctgctggtggctgctccaagatgggtgctgagcgagatctctgaggtgcagctggtggagtccggaggaggactggtgcagcctggaggatccctgagactgagctgcgccgcttctggcttcaacatctactattccagcatgcattgggtgcgccaggctccaggcaagggactggagtgggtggcttacatctcttccagctacggctatacctactatgccgactctgtgaagggcagattcacaatctccgccgataccagcaagaacacagcttatctgcagatgaactccctgagggccgaggacaccgccgtgtactattgtgcccggggctactatgctggctactttggcatcgattattggggccagggcaccctggtgacagtgtcttccgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 60 - DNA, 1400 bp, Artificial Sequence
  Heavy Chain- 1102
atgaagcacctgtggttctttctgctgctggtggctgctccaagatgggtgctgtccgagatcagcgaggtgcagctggtggagagcggaggaggactggtgcagcctggaggcagcctgagactgtcttgcgccgcttccggcttcaacatctactattccagcatgcattgggtgcgccaggctccaggcaagggactggagtgggtggcttctatctactcctattcttcctacaccagctatgccgactctgtgaagggcagattcacaatctccgccgataccagcaagaacacagcttacctgcagatgaactccctgagggccgaggacaccgccgtgtactattgtgcccggtcctttgctatggattattggggccagggcaccctggtgacagtgagctctgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 61 - DNA, 1400 bp, Artificial Sequence
  Heavy Chain- 1103
atgaagcacctgtggttctttctgctgctggtggctgctccaagatgggtgctgtctgagatctccgaggtgcagctggtggagtccggaggaggactggtgcagcctggaggatccctgagactgagctgcgccgcttctggcttcaacatcagctactattctatccattgggtgcgccaggctccaggcaagggactggagtgggtggcctccatctactccagctctggctacacctcctatgccgacagcgtgaagggcaggttcaccatctctgccgatacctccaagaacacagcttatctgcagatgaactccctgagggccgaggacaccgccgtgtactattgtgctcggggctacggcctggattattggggccagggcaccctggtgacagtgtccagcgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 62 - DNA, 1400 bp, Artificial Sequence
  Heavy Chain- 1104
atgaagcacctgtggttctttctgctgctggtggctgctccaagatgggtgctgagcgagatctctgaggtgcagctggtggagtccggaggaggactggtgcagcctggaggatctctgagactgtcctgcgctgcctccggcttcaacatctcctactatagcatccattgggtgcgccaggctccaggcaagggactggagtgggtggcctccatctactccagctcttccagcacctactatgccgactctgtgaagggcaggttcaccatctctgccgatacctccaagaacacagcttatctgcagatgaatagcctgagggccgaggacaccgccgtgtactattgtgctcggggctacggcctggattattggggccagggcaccctggtgacagtgtcttccgctagcaccaagggcccctccgtgttccctctggccccctgtagcagaagcacaagcgagtccaccgccgccctgggctgtctggtgaaggactactttcctgagcctgtgaccgtgtcctggaatagcggcgccctgacctccggcgtgcacacatttcctgccgtgctgcagtcctccggcctgtacagcctgagcagcgtggtgacagtgcccagctccaacttcggcacacagacatacacctgcaatgtggatcacaagccttccaacacaaaggtggacaagaccgtggagagaaagtgttgcgtggagtgccctccttgccccgcccctccagtggctggaccatccgtgttcctgtttccccctaagcccaaggatacactgatgatcagcaggacccccgaggtgacatgcgtggtggtggatgtgagccacgaggaccccgaggtgcagtttaactggtacgtggacggcgtggaggtgcacaacgccaagacaaagcccagggaggagcagttcaattccaccttcagagtggtgagcgtgctgaccgtggtgcaccaggattggctgaatggcaaggagtacaagtgtaaggtgtccaacaagggcctgcccgcccccatcgagaagaccatctccaagacaaagggccagcccagggagccccaggtgtacacactgccccctagcagggaggagatgacaaagaaccaggtgtccctgacctgcctggtgaagggcttctaccctagcgacatcagcgtggagtgggagtccaatggccagcccgagaataactacaagaccaccccccctatgctggatagcgacggcagctttttcctgtactccaagctgacagtggacaagtccaggtggcagcagggcaacgtgtttagctgcagcgtgatgcacgaggccctgcacaaccactacacccagaagagcctgtccctgtcccccggcaagtgagcggccgc

SEQ ID NO: 63 - DNA, 713 bp, Artificial Sequence
  Light Chain- 1095
atggtgctgcagacccaggtgttcatcagcctgctgctgtggatctctggcgcctacggctccgacatccagatgacacagtctccatccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagcccgaggattttgctacatactattgtcagcagcactactccctgttcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 64 - DNA, 713 bp, Artificial Sequence
  Light Chain- 1096
atggtgctgcagacccaggtgttcatcagcctgctgctgtggatctctggcgcctacggctccgacatccagatgacacagtctccttccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagccagaggatttcgccacatactattgtcagcagggctttgctcccttcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 65 - DNA, 713 bp, Artificial Sequence
  Light Chain- 1097
atggtgctgcagacccaggtgttcatcagcctgctgctgtggatctctggcgcctacggctccgacatccagatgacacagtctccttccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagccagaggattttgctacatactattgtcagcagtcctacggccccttcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 66 - DNA, 713 bp, Artificial Sequence
  Light Chain- 1098
atggtgctgcagacccaggtgttcatctccctgctgctgtggatcagcggcgcctacggctctgacatccagatgacacagagcccttccagcctgtctgcctccgtgggcgatagagtgaccatcacatgcagggctagccagtccgtgtcctccgccgtggcttggtatcagcagaagcccggcaaggcccctaagctgctgatctactctgctagctctctgtattctggagtgccatccaggttctccggaagccggtctggaaccgactttaccctgacaatctccagcctgcagccagaggattttgctacatactattgtcagcagtcctacagccccttcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 67 - DNA, 713 bp, Artificial Sequence
  Light Chain- 1099
atggtgctgcagacccaggtgttcatcagcctgctgctgtggatctctggcgcctacggctccgacatccagatgacacagtctccatccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagcccgaggattttgccacatactattgtcagcagcactacgctctgttcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 68 - DNA, 713 bp, Artificial Sequence
  Light Chain- 1100
atggtgctgcagacccaggtgttcatcagcctgctgctgtggatctctggcgcctacggctccgacatccagatgacacagtctccatccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagcccgaggatttcgctacatactattgtcagcagcactacggcctgatcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 69 - DNA, 713 bp, Artificial Sequence
  Light Chain- 1101
atggtgctgcagacccaggtgttcatcagcctgctgctgtggatctctggcgcctacggctccgacatccagatgacacagtctccttccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagccagaggatttcgccacatactattgtcagcaggctggctggcccatcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 70 - DNA, 713 bp, Artificial Sequence
  Light Chain- 1102
atggtgctgcagacccaggtgttcatcagcctgctgctgtggatctctggcgcctacggctccgacatccagatgacacagtctccttccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagccagaggattttgccacatactattgtcagcaggcttactcccccttcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 71 - DNA, 716 bp, Artificial Sequence
  Light Chain- 1103
atggtgctgcagacccaggtgttcatcagcctgctgctgtggatctctggcgcctacggctccgacatccagatgacacagtctccttccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagccagaggatttcgctacatactattgtcagcagcactactcctatcccatcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 72 - DNA, 713 bp, Artificial Sequence
  Light Chain- 1104
atggtgctgcagacccaggtgttcatcagcctgctgctgtggatctctggcgcctacggctccgacatccagatgacacagtctccatccagcctgtctgcctccgtgggcgatagagtgaccatcacatgcagggctagccagtccgtgtcctccgccgtggcttggtatcagcagaagcccggcaaggcccctaagctgctgatctactccgctagctctctgtattccggagtgccaagcaggttctccggaagccggtctggaaccgactttaccctgacaatctccagcctgcagcccgaggatttcgctacatactattgtcagcaggcctacgctctgatcacctttggccagggcacaaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 73 - protein, 5 aa, Artificial Sequence
  Linker
  REPEAT (1)..(5): Repeat n times, where n is an integer of at least 1.
GSGGS

SEQ ID NO: 74 - protein, 4 aa, Artificial Sequence
  Linker
  REPEAT (1)..(4): Repeat n times, where n is an integer of at least 1.
GGGS

SEQ ID NO: 75 - protein, 5 aa, Artificial Sequence
  Linker
  REPEAT (1)..(5): Repeat n times, where n is an integer of at least 1.
GGGGS

SEQ ID NO: 76 - protein, 4 aa, Artificial Sequence
  Linker
GGSG

SEQ ID NO: 77 - protein, 5 aa, Artificial Sequence
  Linker
GGSGG

SEQ ID NO: 78 - protein, 5 aa, Artificial Sequence
  Linker
GSGSG

SEQ ID NO: 79 - protein, 5 aa, Artificial Sequence
  Linker
GSGGG

SEQ ID NO: 80 - protein, 5 aa, Artificial Sequence
  Linker
GGGSG

SEQ ID NO: 81 - protein, 5 aa, Artificial Sequence
  Linker
GSSSG

SEQ ID NO: 82 - protein, 5 aa, Artificial Sequence
  Linker
GGGGS

SEQ ID NO: 83 - protein, 15 aa, Artificial Sequence
  Linker
GGGGSGGGGSGGGGS

SEQ ID NO: 84 - DNA, 45 bp, Artificial Sequence
  Linker
ggtggcggtggctcgggcggtggtgggtcgggtggcggcggatct

(19) United States
(12) Patent Application Publication
(10) Pub. No.: US 2024/0294636 A1
(43) Pub. Date: Sep. 5, 2024

(54) UNC5B FUNCTION BLOCKING ANTIBODIES

(71) Applicants: Yale University, New Haven, CT (US); INSERM (INSTITUT NATIONAL DE LA SANTE ET DE LA RECHERCHE MEDICALE), Paris (FR)

(72) Inventors: Anne Eichmann, Branford, CT (US); Kevin Boyé, New Haven, CT (US); Laurence Pibouin-Fragner, Boulogne-Billancourt (FR); Luiz Geraldo, New Haven, CT (US)

(21) Appl. No.: 18/573,775

(22) PCT Filed: Jun. 27, 2022
(86) PCT No.: PCT/US2022/035137

§ 371 Date: Dec. 22, 2023

Related U.S. Application Data
(60) Provisional application No. 63/215,471, filed on Jun. 27, 2021.

(51) Int. Cl. C07K 16/28; A61K 39/00

(52) U.S. Cl. CPC C07K 16/2803; A61K 2039/505; C07K 2317/24; C07K 2317/55; C07K 2317/76; C07K 2317/92

(57) ABSTRACT

The present disclosure relates to antibodies and binding polypeptides specific for mouse, human, and rat Uncoordinated 5B (Unc5B) protein for use in generating temporary permeability of the blood brain barrier.

### CROSS-REFERENCE TO RELATED APPLICATIONS

The present application claims priority under 35 U.S.C. § 119(e) to U.S. Provisional Patent Application No. 63/215,471, filed Jun. 27, 2021, which is hereby incorporated by reference in its entirety herein.

### STATEMENT REGARDING FEDERALLY SPONSORED RESEARCH OR DEVELOPMENT

This invention was made with government support under grant number HL149343 awarded by the National Institutes of Health. The government has certain rights in the invention.

### SEQUENCE LISTING

The ASCII text file named “047162-7281WO1_Seq Listing,” created Jun. 26, 2022, comprising 97.6 Kbytes, is hereby incorporated by reference in its entirety

### BACKGROUND OF THE DISCLOSURE

The blood brain barrier (BBB) protects the brain from toxins and pathogens, and maintains homeostasis and proper function of the central nervous system (CNS). However, the BBB also impedes treatment of CNS pathologies, because many drugs injected into the circulation cannot reach their targets behind the BBB. The ability to open BBB “on demand,” and to restore its integrity when damaged, has long been a holy grail of therapeutics.

There is a need in the art for developing agents and other treatment strategies that can reversibly permeabilize the BBB. In certain embodiments, such agents and/or compositions comprising same would allow for more effective therapeutic targeting of CNS tissues. The present disclosure addresses this need.

### SUMMARY OF THE DISCLOSURE

As described herein the present disclosure relates in one aspect to antibodies, antigen-binding fragments thereof, and antigen-binding polypeptides specific for mouse, human, and rat Uncoordinated 5B (Unc5B) protein for use in generating temporary permeability of the blood brain barrier.

As such, in one aspect, the invention includes an isolated binding polypeptide comprising an antigen-binding domain that specifically binds to an epitope of human, mouse, and/or rat Unc5B.

In certain preferred embodiments, the antigen-binding domain comprises:

A heavy chain variable region that comprises three heavy chain complementarity determining regions (HCDRs), wherein HCDR1 comprises an amino acid sequence selected from the group comprising SEQ ID NOs: 13-19, HCDR2 comprises an amino acid sequence selected from the group comprising SEQ ID NOs: 20-24, and HCDR3 comprises an amino acid sequence selected from the group comprising SEQ ID NOs: 25-32; and
A light chain variable region that comprises three light chain complementarity determining regions (LCDRs), wherein LCDR1 comprises the amino acid sequence SVSSAVA (SEQ ID NO. 1), LCDR2 comprises the amino acid sequence SASSLYS (SEQ ID NO. 2), and LCDR3 comprises and amino acid sequence selected from the group comprising SEQ ID NOs: 3-12.

In certain embodiments, the binding polypeptide binds an Uncoordinated 5B (Unc5B) protein.

In certain embodiments, the binding polypeptide comprises an antibody or an antigen binding fragment thereof.

In certain embodiments, the antigen-binding fragment is selected from the group consisting of a Fab, a single-chain variable fragment (scFv), and a single-domain antibody.

In certain embodiments, the antibody is a full-length antibody.

In certain embodiments, the antibody or antigen-binding fragment is a humanized antibody or an antigen-binding fragment thereof.

In certain embodiments, the binding polypeptide comprises a heavy chain variable region comprising an amino acid sequence having at least 80%, 85%, 90%, 95%, 96%, 96%, 97%, 98%, or 99% identity to an amino acid sequence of the heavy chain variable region selected from the group comprising SEQ ID NOs: 43-52.

In certain embodiments, the binding polypeptide comprises a heavy chain variable region comprising an amino acid sequence selected from the group comprising SEQ ID NOS: 43-52.

In certain embodiments, the binding polypeptide consists of a heavy chain variable region consisting of an amino acid sequence selected from the group comprising SEQ ID NOs: 43-52.

In certain embodiments, the binding polypeptide comprises a heavy chain variable region encoded by a nucleotide sequence selected from the group comprising SEQ ID NOs: 53-62.

In certain embodiments, the binding polypeptide comprises a light chain variable region comprising an amino acid sequence having at least 80%, 85%, 90%, 95%, 96%, 97%, 98%, or 99% identity to an amino acid sequence selected from the group comprising SEQ ID NOs: 33-42.

In certain embodiments, the binding polypeptide comprises a light chain variable region comprising an amino acid sequence selected from the group comprising SEQ ID NOs: 33-42.

In certain embodiments, the binding polypeptide consists of a light chain variable region comprising an amino acid sequence selected from the group comprising SEQ ID NOs: 33-42.

In certain embodiments, the binding polypeptide comprises a light chain variable region encoded by a nucleotide sequence selected from the group comprising SEQ ID NOS: 63-72.

In another aspect, the invention includes a pharmaceutical composition comprising the isolated binding polypeptide of any of the above aspects or any aspect or embodiment disclosed herein.

In another aspect, the invention includes an isolated nucleic acid encoding a binding polypeptide comprising an antigen-binding domain that specifically binds an epitope of human, mouse, and/or rat Unc5b.

In certain embodiments, the antigen-binding domain comprises:

A heavy chain variable region that comprises three heavy chain complementarity determining regions (HCDRs), wherein HCDR1 comprises an amino acid sequence selected from the group comprising SEQ ID NOs: 13-19, HCDR2 comprises and amino acid sequence selected from the group comprising SEQ ID NOs: 20-24, and HCDR3 comprises an amino acid sequence selected from the group comprising SEQ ID NOs: 25-32; and
A light chain variable region that comprises three light chain complementarity determining regions (LCDRs), wherein LCDR1 comprises the amino acid sequence SVSSAVA (SEQ ID NO. 1), LCDR2 comprises the amino acid sequence SASSLYS (SEQ ID NO. 2), and LCDR3 comprises and amino acid sequence selected from the group comprising SEQ ID NOs: 3-12.

In certain embodiments, the binding polypeptide binds a human, mouse, and/or rat Uncoordinated 5 B protein (Unc5B).

In certain embodiments, the binding polypeptide comprises an antibody or an antigen-binding fragment thereof.

In certain embodiments, the antigen-binding fragment is selected from the group consisting of a Fab, a single-chain variable fragment (scFv), and a single-domain antibody.

In certain embodiments, the antibody is a full-length antibody.

In certain embodiments, the antibody or antigen-binding fragment is a humanized antibody or an antigen-binding fragment thereof.

In certain embodiments, the binding polypeptide comprises a heavy chain variable region comprising an amino acid sequence having at least 80%, 85%, 90%, 95%, 96%, 96%, 97%, 98%, or 99% identity to an amino acid sequence of the heavy chain variable region selected from the group comprising SEQ ID NOs: 43-52.

In certain embodiments, the binding polypeptide comprises a heavy chain variable region comprising an amino acid sequence selected from the group comprising SEQ ID NOS: 43-52.

In certain embodiments, the binding polypeptide consists of a heavy chain variable region consisting of an amino acid sequence selected from the group comprising SEQ ID NOs: 43-52.

In certain embodiments, the binding polypeptide comprises a heavy chain variable region encoded by a nucleotide sequence selected from the group comprising SEQ ID NOs: 53-62.

In certain embodiments, the binding polypeptide comprises a light chain variable region comprising an amino acid sequence having at least 80%, 85%, 90%, 95%, 96%, 97%, 98%, or 99% identity to an amino acid sequence selected from the group comprising SEQ ID NOs: 33-42.

In certain embodiments, the binding polypeptide comprises a light chain variable region comprising an amino acid sequence selected from the group comprising SEQ ID NOs: 33-42.

In certain embodiments, the binding polypeptide consists of a light chain variable region consisting of an amino acid sequence selected from the group comprising SEQ ID NOs: 33-42.

In certain embodiments, the binding polypeptide comprises a light chain variable region encoded by a nucleotide sequence selected from the group comprising SEQ ID NOs: 63-72.

In another aspect, the invention includes a vector comprising the isolated nucleic acid of any one of the above aspects or any other aspect or embodiment disclosed herein.

In certain embodiments, the vector is an expression vector.

In certain embodiments, the vector is selected from the group consisting of a DNA vector, an RNA vector, a plasmid, a lentiviral vector, an adenoviral vector, an adeno-associated viral vector, and a retroviral vector.

In another aspect, the invention includes a host cell comprising the vector of any one of above aspects or any aspect or embodiments disclosed herein.

In certain embodiments, the host cell is of eukaryotic or prokaryotic origin.

In certain embodiments, the host cell is of mammalian origin.

In certain embodiments, the host cell is of bacterial origin.

### BRIEF DESCRIPTION OF THE DRAWINGS

The following detailed description of embodiments of the disclosure will be better understood when read in conjunction with the appended drawings. It should be understood, however, that the disclosure is not limited to the precise arguments and instrumentalities of the embodiments shown in the drawings.

FIGS. 1A-1D illustrate certain newly generated Unc5B blocking antibodies recognize rat, mouse and human Unc5B and decrease Claudin5 protein expression in brain endothelial cells. (FIG. 1A) Unc5b immunoprecipitation on Porcine aortic endothelial cells (PAECs) transfected with rat Unc5B-GFP. (FIG. 1B) Unc5b immunoprecipitation on Mouse Brain Endothelial Cells (Bend.3). (FIG. 1C) Unc5b immunoprecipitation on Human Umbilical Vein Endothelial Cells (HUVEC). (FIG. 1D) Western-blot and protein quantification of Bend3 cells treated with CTRL or Unc5B blocking antibodies 1103 and 1104. All data are shown as mean±SEM.

FIGS. 2A-2D illustrate certain newly generated Unc5B blocking antibodies induce a Blood-Brain Barrier opening for small tracer and nanobodies. (FIGS. 2A-2B) Measurement of real-time TEER using ECISR device on HUVEC and Bend.3 cells treated with CTRL or several Unc5B blocking antibodies. (FIG. 2C) Immunofluorescence of cadaverine and blood vessels (tomato) on brain cortex vibratome sections. Note extravascular cadaverine after 1104 treatment. (FIG. 2D) Immunofluorescence of cadaverine and nanobodies on brain cortex vibratome sections. Note extravascular cadaverine and nanobodies after 1103 treatment.

FIG. 3 is a table of protein sequences of light (LC) and heavy (HC) chains for each generated antibodies.

FIG. 4 is a table of amino acid sequences of light and heavy chains variable regions (respectively CDR-L1 to -L3 and CDR-H1 to -H3) of selected antibodies (variable positions among selected antibodies boxed). CDR-L1 and CDRL2 sequences are identical in all 10 antibodies.

FIGS. 5A-5M are a series of figures demonstrating identification of Unc5B as a regulator of the blood brain barrier.

FIGS. 6A-6I illustrate non-limiting development of antibody blockade of Unc5B.

FIGS. 7A-7J illustrate that the BBB permeability induced by the blockade of Unc5B is temporary and can be reversed by cessation of antibody treatment.

FIGS. 8A-8J illustrates the finding Unc5B regulates the BBB via the Wnt/b-catenin pathway. FIG. 8A: Western blot and quantification of Vegfr2 signaling in adult Unc5Bfl/fl and Unc5BiECko brain protein extracts, n>7 mice per group. FIGS. 8B-8C: Quantification of cadaverine leak in Y949F+/+ adult mice 1 h after blocking antibody injection followed by 30 min of cadaverine injection in the tail vein (n=3 mice per group). FIG. 8D: Western blot and quantification (FIG. 8E) of Wnt signaling components in adult Unc5Bfl/fl and Unc5BiECko brain protein extracts, n>7 mice per group. FIGS. 8F-8G: Immunofluorescence and quantification of LEF1 staining on adult brain vibratome sections (150 μm). FIG. 8H: Western blot for Claudin5 and quantification of adult Unc5Bfl/fl, Unc5BiECko and Unc5BiECkoCtnnb1Flex/3 brain protein extracts. FIG. 8I: Cadaverine leak quantification of adult Unc5BiECko and Unc5BiECkoCtnnb1Flex/3 mice 30 min after dye injection in the tail-vein (n=5 mice per group). FIG. 8J: Non-limiting working model. All data are shown as mean±SEM. NS: non-significant, Mann-Whitney U test was performed for statistical analysis.

FIG. 9A: Diagram illustrating generation of the Unc5B Flox allele. FIG. 9B: qPCR analysis of all Unc5B exons on P12 lung endothelial cells, n>5 mice/group. FIG. 9C: Western blot and quantification of P5 Unc5Bfl/fl and Unc5BiECko brain protein extracts, n>7 animals/group. All data are shown as mean±SEM. NS: non-significant, Mann-Whitney U test was performed for statistical analysis.

FIG. 10A: Adult WT brain staining with the indicated antibodies on 150 μm vibratome sections. FIG. 10B: Adult Unc5Bfl/fl and Unc5BiECko brain staining for Unc5B and endomucine on 150 μm vibratome sections performed in several brain areas (cortex, hippocampus, striatum and cerebellum). Note absence of Unc5B staining in Unc5BiECko brain after TAM injection in all brain regions observed.

FIG. 11A: Whole-mount retina staining with IB4 along with several BBB components (PDGFRb, GFAP and Claudin5) on P5 Unc5Bfl/fl and Unc5BiECko. FIG. 11B: P5 Unc5Bfl/fl and Unc5BiECko immunofluorescence on 10 μm cerebral cortex section or whole-mount retina. FIG. 11C: P5 brain brightfield imaging after cadaverine tracer injection.

FIG. 12A: Immunofluorescence of blood vessels and endogenous IgG and fibrinogen on 150 μm cortex vibratome sections after antibody injection. FIG. 12B: 10 kDa dextran leak quantification on adult Unc5Bfl/fl and Unc5BiECko mice 30 min after dye injection into the tail-vein (n>4 mice/group). FIG. 12C: Immunofluorescence of cadaverine and blood vessels on 150 μm vibratome sections. Note presence of extravascular cadaverine in several brain regions in anti-Unc5B-2 injected mice. All data are shown as mean±SEM. NS: non-significant, Mann-Whitney U test was performed for statistical analysis. Mann-Whitney U test was performed for statistical analysis.

FIG. 13A: Unc5B global KO embryos were isolated at E12.5. FIG. 13B: qPCR analysis on E12.5 brain mRNA extracts, n=6 embryos/group. FIG. 13C: Western blot and quantification of E12.5 Unc5B WT and KO brain protein extracts, n=10 embryos/group.

FIG. 13D: Whole-mount hindbrain immunofluorescence of Endomucin and Claudin5 on E12.5 Unc5B WT and KO embryos. FIG. 13E: Western-blot and protein quantification of Bend3 cells treated with SiCTRL or siUnc5B, n=7. FIG. 13F: Western-blot and protein quantification of HUVEC cells infected with CTRL or Unc5B full length adenovirus. Note the increased Claudin5 expression after Unc5B overexpression, n=3. All data are shown as mean±SEM. NS: non-significant. Mann-Whitney U test was performed for statistical analysis between two groups. ANOVA followed by Bonferroni's multiple comparisons test was performed for statistical analysis between 3 groups.

FIG. 14: Western blot of P5 WT BI/6 brain protein extract 1 hour after blocking antibody injection (10 mg/kg). All data are shown as mean±SEM. NS: non-significant. Mann-Whitney U test was performed for statistical analysis between two groups.

FIG. 15A: Western blot and protein quantification of brain protein lysates from eGFP::Claudin5 mice. FIG. 15B: Immunofluorescence of blood vessels and eGFP-Cldn5 transgene on 150 μm cortex vibratome sections.

FIG. 16A: Western blot and protein quantification of brain protein lysates from Y949F mice. FIG. 16B: Immunofluorescence of cadaverine and blood vessels upon blocking antibody treatment. Note extravascular cadaverine in both anti-Unc5B-2 injected WT and Y949F mice. All data are shown as mean±SEM. NS: non-significant, ANOVA followed by Bonferroni's multiple comparisons test was performed for statistical analysis between 3 groups.

FIG. 17A: qPCR analysis on brain mRNA extracts from Unc5Bfl/fl and Unc5BiECko mice, n>4 mice/group. FIGS. 17B-17C: Western-blot and protein quantification of Bend3 cells treated with siCTRL or siUnc5B, n>4. All data are shown as mean±SEM. NS: non-significant, Mann-Whitney U test was performed for statistical analysis between two groups.

FIG. 18: Anti-Unc5B antibody 1103 induces selected BBB opening to tracer molecules up to 40 kDa.

FIG. 19: Treatment with anti-Unc5B antibody 1103 does not increase leak of the tracer into other organs, including skin, heart, kidney, and lung.

FIG. 20: Treatment with anti-Unc5B antibody 1103 does not increase leak of the 40 kDa dextran tracer into other organs, including skin, heart, kidney, and lung.

FIG. 21: Treatment with anti-Unc5B antibody 1103 allows nanobody transport across the BBB (far left), but not into other tissues.

FIG. 22: Treatment with anti-Unc5B antibody 1103 allows the transport of growth factor molecules, brain-derived neurotrophic factor (BDNF) in this case, across the BBB.

FIG. 23: Affinities of the Unc5B antibodies of the invention as determined by biacore analyses.

FIGS. 24A-24D: Netrin-1 binding to Unc5B mediates BBB integrity. Western-blot (FIG. 24A) and quantification (FIG. 24B) of ECs treated with CTRL IgG or anti-Unc5B-3 for 1 h followed by recombinant mouse Netrin-1 treatment (500 ng/ml) for 10 min or 30 min. Each dot represents one independent experiment, n=4 independent experiment. FIG. 24C. Unc5B immunoprecipitation with a commercial antibody (R&D systems) of brain protein extracts from mice i.v injected with CTRL or anti-Unc5B-3 antibodies (1 h, 10 mg/kg), and western blot with antibodies recognizing the indicated ligands. FIG. 24D. Quantification of h, n=5 CTRL anti-Unc5B-1 and n=5 anti-Unc5B-3 treated animals. Each dot represents one mouse. One control mouse was set as 1.

### DETAILED DESCRIPTION

The present disclosure is based, in one aspect, on the unexpected finding that uncoordinated 5B (Unc5B) is a protein highly expressed on endothelial cells in humans and rodents, especially on cells forming the blood brain barrier. In some embodiments of the present disclosure, inhibition of Unc5B function through genetic, pharmacologic, and/or antibody-based strategies can be used to induce transient permeability of the blood brain barrier (BBB). The BBB prevents the brain uptake of most pharmaceuticals. This property arises from the epithelial-like tight junctions within the brain capillary endothelium. Small molecule drugs may cross the BBB via lipid-mediated free diffusion, only if the drug has a molecular weight of 400 Da and forms hydrogen bonds. These chemical properties are lacking in >90% of small molecule drugs, and all large molecule drugs. The ability to open BBB “on demand” and to restore its integrity when damaged, has long been a desired goal of therapeutic design and development.

Uncoordinated 5 (Unc5) was initially discovered in a C. elegans screen for motor dysfunctions, and controls axon guidance in response to its ligand Netrin in all species examined. The vertebrate Unc5 family contains four homologues, Unc5A-D. Among those, only Unc5B is expressed in endothelial cells in mice and humans. Global Unc5B knockout in mice is embryonically lethal at mid-gestation due to vascular defects, demonstrating that Unc5B has important functions in vascular development. Whether this receptor is required for later stages of vascular development and homeostasis has remained unknown. In this disclosure, the endothelial Unc5B receptor is identified as a novel regulator of BBB integrity that can be targeted both genetically and pharmacologically with blocking antibodies to open BBB tight junctions in mice. In certain embodiments, the current disclosure includes novel monoclonal Unc5B antibodies that can be used to open the BBB.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice of and/or for the testing of the present disclosure, illustrative materials and methods are described herein. In describing and claiming the present disclosure, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instance ±0.1% from the specified value, as such variations are appropriate to perform the disclosed methods.

The term “antibody,” as used herein, refers to an immunoglobulin molecule capable of binding to an antigen. Antibodies can be intact immunoglobulins derived from natural sources or from recombinant sources and can be antigen-binding portions of intact immunoglobulins. Antibodies are typically tetramers of immunoglobulin molecules. The antibody may exist in a variety of forms where the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY: Harlow et al., 1989, In: Antibodies: A Laboratory Manual, Cold Spring Harbor, New York: Houston et al., 1988, Proc. Natl. Acad. Sci. USA 85:5879-5883; Bird et al., 1988, Science 242:423-426). As used herein, “Fab” refers to a fragment of an antibody structure that binds to an antigen but is monovalent and does not have an Fc portion, for example, an antibody digested by the enzyme papain yields two Fab fragments and an Fc fragment (e.g., a heavy (H) chain constant region designated as an Fc fragment that does not bind antigen). As used herein, “F(ab′)2” refers to an antibody fragment generated by pepsin digestion of whole IgG antibodies having two antigen-binding (ab′) (bivalent) regions, each (ab′) region comprising two separate amino acid chains, a part of a heavy (H) chain and a light (L) chain linked by a disulfide (S—S) bond which maintains the structure of the binding pocket for binding an antigen and where the remaining H chain portions are linked together. A “F(ab′)2” fragment can be split into two individual Fab′ fragments.

The term “high affinity” as used herein refers to a strong interaction of one molecule with a target molecule with K_{D} values in the nanomolar (10⁻⁹, 10⁻¹⁰) range. High affinity antibodies are generally considered to be in the low nanomolar range (10⁻⁹) with very high affinity antibodies being in the picomolar (10⁻¹²) range. Biacore analysis of binding affinity for Abs 1103 and 1097 reveal a KD of 1.3 nM (1103) and 700 pM (1097).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Effective amount” or “therapeutically effective amount” are used interchangeably herein, and refer to an amount of a compound, formulation, material, or composition, as described herein effective to achieve a particular biological result.

The term “effector function” refers to a specialized function of an immune cell.

As used herein, the term “endogenous” refers to substances or processes that originate from within a particular system, such as an organism, tissue, or cell. In contrast, the term “exogenous” as used herein refers to substances or processes that originate from outside a particular system, such as an organism, tissue, or cell.

The term “expression” as used herein is defined as the transcription and/or translation of a particular nucleotide sequence driven by a promoter.

“Expression vector” refers to a vector comprising one or more recombinant polynucleotide sequences comprising expression control elements operatively linked to a nucleotide sequence to be expressed. An expression vector comprises sufficient cis-acting elements for gene expression: other elements for expression can be supplied by the host cell or in an in vitro expression system. Expression vectors include all those known in the art, such as cosmids, plasmids (e.g., naked or contained in liposomes), retrotransposons (e.g. piggy back, sleeping beauty), and viruses (e.g., lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions: e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions: e.g., if half (e.g., five positions in a polymer ten amino acids in length) of the positions in two sequences are identical, the two sequences are 50% identical: if 90% of the positions (e.g., 9 of 10), are matched or identical, the two amino acids sequences are 90% identical.

The term “inhibition” refers to a primary response induced by binding of an inhibitory molecule with its cognate ligand, thereby mediating a signal transduction event that attenuates, terminates, or opposes biological outcomes.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “limited toxicity” as used herein, refers to the peptides, polynucleotides, cells and/or antibodies of the disclosure manifesting a lack of negative biological effects, anti-tumor effects, or negative physiological symptoms toward a healthy cell, non-tumor cell, non-diseased cell, non-target cell or population of such cells either in vitro or in vivo.

A “lentivirus” as used herein refers to a genus of the Retroviridae family. Lentiviruses are unique among the retroviruses in being able to infect non-dividing cells: they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. In some embodiments, a nucleic acid sequence is considered to have at least 95%, 96%, 97%, 98%, or 99% identity or homology to any nucleic acid sequence disclosed herein.

As used herein, the terms “peptide,” “polypeptide,” and “protein” are used interchangeably to refer to a polymer of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. In some embodiments, an amino acid sequence is considered to have at 95%, 96%, 97%, 98%, or 99% identity or homology to any amino acid sequence described herein.

The term “promoter” refers to a regulatory region of DNA located near a gene, providing a control point for regulated gene transcription. A promoter comprises specific DNA sequences recognized by proteins known as transcription factors that bind to the promoter sequence and recruit RNA polymerase, the enzyme that performs template-directed synthesis of RNA from the coding strand of the DNA.

A “tissue-specific” promoter is a nucleotide sequence which, when operably linked to a polynucleotide encoding a gene product, causes the gene product to be produced in a cell substantially only if the cell is a cell of the tissue type corresponding to the promoter.

By the term “specifically binds,” as used herein, refers to an antibody, a receptor, or a ligand capable of recognizing and binding to a cognate binding partner present in a sample, but which antibody or ligand does not substantially recognize or bind other molecules in the sample.

The term “subject” as used herein refers to any living organism in which an adaptive immune response can be elicited (e.g., mammals). In a non-limiting aspect, the subject is a human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with one or more exogenous nucleic acid molecules. The term “cell” in this context includes the primary subject cell and its progeny.

“Transmembrane domain” refers to a portion or a region of a molecule that spans a lipid bilayer membrane.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Binding Polypeptides, Antibodies, and scFvs

The binding polypeptides and antibodies of the disclosure are characterized by particular functional features or properties of the antibodies. For example, the binding polypeptides and antibodies specifically bind to Unc5B. In certain embodiments, the binding polypeptides and antibodies of the disclosure bind to mouse, rat, and/or human Unc5B with high affinity. In certain embodiments, the binding polypeptides and antibodies of the disclosure specifically recognize naturally expressed canine FAP protein on a cell and do not cross-react to other surface molecules on that cell.

In certain aspect, the disclosure provides an isolated binding polypeptide comprising an antigen-binding domain that specifically binds to an epitope of human, rat, and/or murine Unc5B extracellular domain. In certain embodiments, the antigen-binding domain comprises a heavy chain variable region that comprises three heavy chain complementarity determining regions (HCDRs) and a light chain variable region that comprises three light chain complementarity determining regions (LCDRs).

In certain aspect, the disclosure provides an isolated binding polypeptide comprising an HCDR1 comprising an amino acid sequence selected from the group consisting of FNISYSSIHW (SEQ ID NO: 13), FNLYYSSIHW (SEQ ID NO: 14), FNLYYYSMHW (SEQ ID NO: 15), FNIYYSYMHW (SEQ ID NO: 16), FNLYYYSIHW (SEQ ID NO: 17), FNIYYSSMHW (SEQ ID NO: 18), and FNISSYYSIHW (SEQ ID NO: 19).

Also provided is an isolated binding polypeptide comprising an HCDR2 comprising an amino acid sequence selected from the group consisting of AYIYPYYGYTY (SEQ ID NO: 20), ASIYSYSSYTS (SEQ ID NO: 21), ASIYPYSSYTS (SEQ ID NO: 22), ASIYSSSGYTS (SEQ ID NO: 23), ASIYSSSSSTY (SEQ ID NO: 24).

Also provided is an isolated binding polypeptide comprising an HCDR3 comprising an amino acid sequence selected from the group consisting of RGYGIDY (SEQ ID NO: 25), RSYAMDY(SEQ ID NO: 26), RHYAMDY (SEQ ID NO: 27), RGYAFNY (SEQ ID NO: 28), RGYAMDY (SEQ ID NO: 29), RGYYAGYFGIDY (SEQ ID NO: 30), RSFAMDY (SEQ ID NO: 31), and RGYGLDY (SEQ ID NO: 32).

Also provided is an isolated binding polypeptide comprising a light chain variable region that comprises an LCDR1 comprising the amino acid sequence SVSSAVA (SEQ ID NO: 1).

Also provided is an isolated binding polypeptide comprising an LCDR2 comprising the amino acid sequence SASSLYS (SEQ ID NO: 2).

Also provided is an isolated binding polypeptide comprising an LCDR3 comprising an amino acid sequence selected from the group consisting of QHYSLF (SEQ ID NO: 3), QGFAPF (SEQ ID NO: 4), QSYGPF (SEQ ID NO: 5), QSYSPF (SEQ ID NO: 6), QHYALF (SEQ ID NO: 7), QHYGLI (SEQ ID NO: 8), QAGWPI (SEQ ID NO: 9), QAYSPF (SEQ ID NO: 10), QHYSYPI (SEQ ID NO: 11), and QAYALI (SEQ ID NO: 12).

Tolerable variations of the complementarity determining regions (CDR) sequences will be known to those of skill in the art. For example, in some embodiments the polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

In some embodiments, the binding polypeptide binds an Unc5B protein. In some embodiments, the binding polypeptide comprises an antibody or an antigen-binding fragment thereof. In some embodiments, the antigen-binding fragment is selected from the group consisting of a Fab, a single-chain variable fragment (scFv), or a single-domain antibody. In further embodiments, the antibody is a full-length antibody. In yet further embodiments, the antibody or antigen-binding fragment is a mouse antibody or an antigen-binding fragment thereof. In some embodiments, the antibody or antigen-binding fragment is a humanized antibody or an antigen-binding fragment thereof.

In certain embodiments, the binding polypeptide comprises a heavy chain variable region comprising an amino acid sequence having at least 80%, 85%, 90%, 95%, 96%, 96%, 97%, 98%, 99% identity to the amino acid sequence of the heavy chain variable region set forth in SEQ ID NOs: 43-52. In certain embodiments, the binding polypeptide comprises a heavy chain variable region comprising an amino acid sequence set forth in SEQ ID NOs: 43-52. In certain embodiments, the binding polypeptide consists of a heavy chain variable region consisting of an amino acid sequence set forth in SEQ ID NOs: 43-52.

In certain embodiments, the binding polypeptide comprises a light chain variable region comprising an amino acid sequence having at least 80%, 85%, 90%, 95%, 96%, 97%, 98%, 99% identity to the amino acid sequence set forth in SEQ ID NOs: 33-42. In certain embodiments, the binding polypeptide comprises a light chain variable region comprising an amino acid sequence set forth in SEQ ID NOs: 33-42. In certain embodiments, the binding polypeptide consists of a light chain variable region comprising an amino acid sequence set forth in SEQ ID NOs: 33-42.

In certain embodiments, the disclosure includes an antibody that binds to the same epitope on human, mouse, or rat Unc5B as an antibody of the disclosure (i.e., antibodies that have the ability to cross-compete for binding to human Unc5B with any of the antibodies of the disclosure). In certain embodiments, the reference antibody for cross-competition studies can be one of the antibodies described herein (for example 1103). For example, Biacore analysis, ELISA assays or flow cytometry may be used to demonstrate cross-competition with the antibodies of the current disclosure. The ability of a test antibody to inhibit the binding of, for example, 1103, to human Unc5B demonstrates that the test antibody can compete with 1103 for binding to rat, mouse, and human Unc5B and thus is considered to bind to the same epitope of Unc5B as 1103.

An antibody of the disclosure can be prepared using an antibody having one or more of the VH and/or VL sequences disclosed herein as a starting material to engineer a modified antibody, which modified antibody may have altered properties as compared with the starting antibody. An antibody can be engineered by modifying one or more amino acids within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

In some embodiments of the disclosure, the antigen-binding domain is a single-chain variable fragment (scFv) that specifically binds to an epitope of human, rat, and/or murine Unc5B.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., FAP binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present disclosure.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6): 1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO:73), (GGGS)ₙ (SEQ ID NO:74), and (GGGGS)ₙ (SEQ ID NO:75), where n represents an integer of at least 1. Exemplary linker sequences can comprise amino acid sequences including, without limitation, GGSG (SEQ ID NO:76), GGSGG (SEQ ID NO:77), GSGSG (SEQ ID NO:78), GSGGG (SEQ ID NO:79), GGGSG (SEQ ID NO:80), GSSSG (SEQ ID NO:81), GGGGS (SEQ ID NO:82), GGGGSGGGGSGGGGS (SEQ ID NO:83) and the like. Those of skill in the art would be able to select the appropriate linker sequence for use in the present disclosure. In one embodiment, an scFv of the present disclosure comprises a heavy chain variable region (VH) and a light chain variable region (VL), wherein the VH and VL is separated by the linker sequence having the amino acid sequence GGGGSGGGGSGGGGS (SEQ ID NO:83), which may be encoded by the nucleic acid sequence

(SEQ ID NO: 84)
GGTGGCGGTGGCTCGGGCGGTGGTGGGTCGGGTGGCGGCGGATCT.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51: Peter et al., J Cachexia Sarcopenia Muscle 2012 Aug. 12: Shieh et al., J Imunol 2009 183(4):2277-85: Giomarelli et al., Thromb Haemost 2007 97(6):955-63; Fife et al., J Clin Invst 2006 116(8):2252-61: Brocks et al., Immunotechnology 1997 3(3): 173-84: Moosmayer et al., Ther Immunol 1995 2(10:31-40). Agonistic scFvs having stimulatory activity have been described (see, e.g., Peter et al., J Bioi Chem 2003 25278(38):36740-7: Xie et al., Nat Biotech 1997 15(8): 768-71: Ledbetter et al., Crit Rev Immunol 1997 17(5-6):427-55: Ho et al., BioChim Biophys Acta 2003 1638(3):257-66).

### Nucleic Acids and Expression Vectors

The present disclosure provides an isolated nucleic acid encoding a polypeptide. The nucleic acid of the present disclosure may comprise a polynucleotide sequence encoding any one of the binding polypeptides, scFv, or antibodies disclosed herein.

One aspect of the disclosure includes an isolated nucleic acid encoding a binding polypeptide comprising an antigen-binding domain that specifically binds an epitope of human, rat, and/or murine Unc5B extracellular domain.

In certain embodiments, the nucleic acid comprises an antigen binding domain comprising a heavy chain variable region that comprises three heavy chain complementarity determining regions (HCDRs) and a light chain variable region that comprises three light chain complementarity determining regions (LCDRs). In certain embodiments, the HCDR1 comprises one of the amino acid sequences FNISYSSIHW (SEQ ID NO: 13), FNLYYSSIHW (SEQ ID NO: 14), FNLYYYSMHW (SEQ ID NO: 15), FNIYYSYMHW (SEQ ID NO: 16), FNLYYYSIHW (SEQ ID NO: 17), FNIYYSSMHW (SEQ ID NO: 18), and FNISSYYSIHW (SEQ ID NO: 19), In certain embodiments, the HCDR2 comprises one of the amino acid sequences AYIYPYYGYTY (SEQ ID NO: 20), ASIYSYSSYTS (SEQ ID NO: 21), ASIYPYSSYTS (SEQ ID NO: 22), ASIYSSSGYTS (SEQ ID NO: 23), ASIYSSSSSTY (SEQ ID NO: 24), In certain embodiments, the HCDR3 comprises one of the amino acid sequence RGYGIDY (SEQ ID NO: 25), RSYAMDY(SEQ ID NO: 26), RHYAMDY (SEQ ID NO: 27), RGYAFNY (SEQ ID NO: 28), RGYAMDY (SEQ ID NO: 29), RGYYAGYFGIDY (SEQ ID NO: 30), RSFAMDY (SEQ ID NO: 31), and RGYGLDY(SEQ ID NO: 32), In certain embodiments, the LCDR1 comprises the amino acid sequence SVSSAVA (SEQ ID NO: 1), In certain embodiments, the LCDR2 comprises the amino acid sequence SASSLYS (SEQ ID NO: 2), In certain embodiments, the LCDR3 comprises one of the amino acid sequences QHYSLF (SEQ ID NO: 3), QGFAPF (SEQ ID NO: 4), QSYGPF (SEQ ID NO: 5), QSYSPF (SEQ ID NO: 6), QHYALF (SEQ ID NO: 7), QHYGLI (SEQ ID NO: 8), QAGWPI (SEQ ID NO: 9), QAYSPF (SEQ ID NO: 10), QHYSYPI (SEQ ID NO: 11), and QAYALI (SEQ ID NO: 12).

In certain embodiments, the binding polypeptide comprises an antibody or an antigen-binding fragment thereof. In certain embodiments, the antigen-binding fragment is selected from the group consisting of a Fab, a single-chain variable fragment (scFv), or a single-domain antibody. In certain embodiments, the antibody is a full-length antibody. In certain embodiments, the antibody or antigen-binding fragment is a humanized antibody or a fragment thereof.

In certain embodiments, the heavy chain variable region is encoded by a nucleic acid comprising a polynucleotide sequence having at least 80%, 85%, 90%, 95%, 96%, 96%, 97%, 98%, 99% identity to one of SEQ ID NOs: 43-52. In certain embodiments, the heavy chain variable region is encoded by a nucleic acid comprising one of the polynucleotide sequences set forth in SEQ ID NOs: 43-52. In certain embodiments, the heavy chain variable region is encoded by a nucleic acid consisting of one of the polynucleotide sequences set forth in SEQ ID NOs: 43-52. In certain embodiments, the heavy chain variable region is encoded by a nucleic acid comprising one of the sequences set forth in SEQ ID NOs: 53-62.

In certain embodiments, the light chain variable region is encoded by a nucleic acid comprising a polynucleotide sequence having at least 80%, 85%, 90%, 95%, 96%, 96%, 97%, 98%, 99% identity to the amino acid sequences of one of the light chain variable regions set forth in SEQ ID NOs: 33-42. In certain embodiments, the light chain variable region is encoded by a nucleic acid comprising one of the polynucleotide sequences set forth in SEQ ID NOs: 33-42. In certain embodiments, the light chain variable region is encoded by a nucleic acid consisting of one of the polynucleotide sequences set forth in SEQ ID NOs: 33-42. In certain embodiments, the light chain variable region is encoded by a nucleic acid comprising one of the sequences set forth in SEQ ID NOs: 63-72.

Also provided is an isolated nucleic acid encoding a binding polypeptide comprising a heavy chain variable region encoded by a nucleic acid sequence comprising one of the polynucleotide sequences set forth in SEQ ID NOs: 43-52 and one of the nucleic acid sequences set forth in SEQ ID NOs: 53-62, and a light chain variable region encoded by a nucleic acid sequence comprising one of the polynucleotide sequences set forth in SEQ ID NO: 33-42 and one of the nucleic acid sequences set forth in SEQ ID NOs: 63-72.

Another aspect of the disclosure provides a vector comprising any one of the isolated nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell may be of eukaryotic, prokaryotic, mammalian, or bacterial origin. A method of producing a binding polypeptide that binds to Unc5B is also provided herein, wherein the method comprises culturing the host cell.

In some embodiments, a nucleic acid of the present disclosure may be operably linked to a transcriptional control element, e.g., a promoter, enhancer, and so forth. Suitable promoter and enhancer elements are known to those of skill in the art.

In certain embodiments, the nucleic acid is in operable linkage with a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements: cytomegalovirus immediate early promoter: herpes simplex virus thymidine kinase promoter: early and late SV40 promoters: promoter present in long terminal repeats from a retrovirus: mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soy bean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used: see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter: see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like: or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter: a trp promoter: a lac operon promoter: a hybrid promoter, e.g., a lac/tac hybrid promoter, a tac/trc hybrid promoter, a trp/lac promoter, a T7/lac promoter: a trc promoter: a tac promoter, and the like: an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141: Mckelvie et al., Vaccine (2004) 22:3243-3255; and Chatfield et al., Biotechnol. (1992) 10:888-892): a sigma70 promoter, e.g., a consensus sigma70) promoter (see, e.g., GenBank Accession Nos. AX798980, AX798961, and AX798183): a stationary phase promoter, e.g., a dps promoter, an spy promoter, and the like: a promoter derived from the pathogenicity island SPI-2 (see, e.g., WO96/17951): an actA promoter (see, e.g., Shetron-Rama et al., Infect. Immun. (2002) 70:1087-1096): an rpsM promoter (see, e.g., Valdivia and Falkow Mol. Microbiol. (1996). 22:367): a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162): an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has 30) a conformation that binds the operator: in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp. Molecular Biology (2012) (Jones & Bartlett Publishers, Sudbury, Mass.), the disclosures of which are incorporated herein by reference.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art: many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA): pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus: poliovirus: adenovirus (see, e.g., Li et al., Invest. Opthalmol. Vis. Sci. (1994) 35: 2543-2549; Borras et al., Gene Ther. (1999) 6: 515-524: Li and Davidson, Proc. Natl. Acad. Sci. USA (1995) 92: 7700-7704; Sakamoto et al., H. Gene Ther. (1999) 5: 1088-1097: WO 94/12649, WO 93/03769; WO 93/19191: WO 94/28938: WO 95/11984 and WO 95/00655): adeno-associated virus (see, e.g., Ali et al., Hum. Gene Ther. (1998) 9: 81-86, Flannery et al., Proc. Natl. Acad. Sci. USA (1997) 94: 6916-6921: Bennett et al., Invest. Opthalmol. Vis. Sci. (1997) 38: 2857-2863: Jomary et al., Gene Ther. (1997) 4:683 690, Rolling et al., Hum. Gene Ther. (1999) 10: 641-648; Ali et al., Hum. Mol. Genet. (1996) 5: 591-594: Srivastava in WO 93/09239, Samulski et al., J. Vir. (1989) 63: 3822-3828: Mendelson et al., Virol. (1988) 166: 154-165; and Flotte et al., Proc. Natl. Acad. Sci. USA (1993) 90: 10613-10617): SV40; herpes simplex virus: human immunodeficiency virus (see, e.g., Miyoshi et al., Proc. Natl. Acad. Sci. USA (1997) 94: 10319-23: Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the nucleic acid into a host cell. Accordingly, an expression vector (e.g., a lentiviral vector) of the present disclosure may comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In one embodiment, the vector further comprises an elongation-factor-1-alpha promoter (EF-1α promoter). Use of an EF-1α promoter may increase the efficiency in expression of downstream transgenes. Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present disclosure. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present disclosure. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present disclosure further comprises a WPRE sequence. Various posttranscriptional regulator elements are known to those of skill in the art and may be incorporated into a vector (e.g., lentiviral vector) of the present disclosure. A vector of the present disclosure may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present disclosure includes a 3′ U3 deleted LTR. Accordingly, a vector (e.g., lentiviral vector) of the present disclosure may comprise any combination of the elements described herein to enhance the efficiency of functional expression of transgenes. For example, a vector (e.g., lentiviral vector) of the present disclosure may comprise a WPRE sequence, cPPT sequence, RRE sequence, 5′LTR, 3′ U3 deleted LTR′ in addition to a nucleic acid encoding for a CAR.

Vectors of the present disclosure may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present disclosure may be RNA, e.g., in vitro synthesized RNA. Methods for in vitro synthesis of RNA are known to those of skill in the art: any known method can be used to synthesize RNA comprising a sequence encoding a polypeptide of the present disclosure. Methods for introducing RNA into a host cell are known in the art. See, e.g., Zhao et al. Cancer Res. (2010) 15: 9053. Introducing RNA comprising a nucleotide sequence encoding a polypeptide of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of a polypeptide as described herein, e.g., in a host cell. In some embodiments, a nucleic acid of the present disclosure provides for amplification of the polypeptide-encoding nucleic acid.

### Methods of Treatment

The antibodies and binding polypeptides described herein may be included in a composition for treating, ameliorating, and/or preventing a disease or condition in a subject in need thereof. In certain embodiments, the subject is a human. The composition may include a pharmaceutical composition and further include a pharmaceutically acceptable carrier. A therapeutically effective amount of the pharmaceutical composition may be administered to the subject.

In certain embodiments, the method comprises administering to the subject an isolated binding polypeptide comprising a heavy chain variable region comprising an amino acid sequence at least 80%, 85%, 90%, 95%, 96%, 97%, 98%, 99%, or 100% identical to one of SEQ ID NOs: 43-52 and a light chain variable region comprising an amino acid sequence at least 80%, 85%, 90%, 95%, 96%, 97%, 98%, 99%, or 100% identical to one of SEQ ID NOs: 33-42, or any other constructs contemplated herein.

In certain embodiments, the use of Unc5B binding polypeptides and antibodies induces a temporary permeability to the BBB in a subject need thereof. The BBB protects the brain from toxins and pathogens, and maintains homeostasis and proper function of the central nervous system (CNS). However, the BBB also impedes treatment of CNS pathologies, because many drugs injected into the circulation cannot reach their targets behind the BBB. The ability to open BBB “on demand” and to restore its integrity when damaged, has long been a holy grail of therapeutics. In certain embodiments, the antibodies and binding polypeptides of the disclosure bind Unk5B to cause transient openings in the tight junctions of the BBB in the subject.

Compositions of the disclosure can be administered in dosages and routes and at times to be determined in appropriate pre-clinical and clinical experimentation and trials. Compositions may be administered multiple times at dosages within these ranges. Administration of the compositions may be combined with other methods useful to treat the desired disease or condition as determined by those of skill in the art.

### Pharmaceutical Compositions and Formulations

Also provided are pharmaceutical composition comprising any one of the binding polypeptides, scFvs, antibodies, or the antigen-binding fragments disclosed herein. Among the compositions are pharmaceutical compositions and formulations for administration, such as for treatment of a disease or disorder. Also provided are therapeutic methods for administering the pharmaceutical compositions to subjects, e.g., patients.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular composition and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids: antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone: amino acids such as glycine, glutamine, asparagine, histidine, arginine, or lysine: monosaccharides, disaccharides, and other carbohydrates including glucose, mannose, or dextrins: chelating agents such as EDTA: sugars such as sucrose, mannitol, trehalose or sorbitol: salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins: 21st ed. (May 1, 2005).

The formulations can include aqueous solutions. The formulation or composition may also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in an amount effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

The materials and methods are now described.

### Mouse Models:

Generation of the targeted Unc5b allele was performed by homologous recombination in RI ES cells. Correctly targeted cells were identified by Southern blot hybridization and injected into B6J blastocysts to generate Unc5bneo/+ mice. To remove the neo cassette, Unc5bneo/+ mice were mated to B6.129S4-Gt(ROSA)26Sortm1(FLP1)Dym/RainJ mice (The Jackson Laboratory, stock #009086). Mice were backcrossed to B6J mice for ten generations. Unc5Bfl/fl (B6-Unc5b<tml(flox)Slac/Slac) mice were then bred with Cdh5-CreERT2 mice10). eGFP::Claudin5 transgenic mice, Y949F mice, and bcatenin GOF Ctnnb1flex/3 mice were described previously (Knowland, et al., 2014, Neuron 82:603-617: Li, et al., 2016, Nat Commun 7:11017; Harada, et al., 1999, EMBO J 18:5931-5942). Gene deletion was induced by injection of tamoxifen (Sigma T5648) diluted in corn oil (Sigma C8267). Postnatal gene deletion was induced by 3 injections of 100 ug of tamoxifen at P0, P1 and P2: whereas adult gene deletion was induced by 5 injections of 2 mg of tamoxifen from P60 to P65.

### Cell Culture:

Bend3 cells were purchased from ATCC (ATCCR CRL-2299™) and cultured in Dulbecco's Modified Eagle's Medium (DMEM) (ATCCR: 30-2002™) supplemented with 10% fetal bovine serum (FBS) and 1% Penicillin Streptomycin. Cells were cultured at 37° C. and 5% CO2.

### Small Interfering RNA Knockdown Experiments:

For Unc5B Inhibition, cells were transiently transfected with siRNA (Dharmacon). ONTARGETplus Mouse Unc5b siRNA (SMARTpool, L-050737-01-0005) were used for Unc5B gene deletion in Bend3 cells. Transfection was performed using lipofectamine RNAi max (Invitrogen, 13778-075) according to the manufacturer's instruction with siRNA at a final concentration of 25pmol in OptiMem for 8h. After transfection, cells were washed with PBS and fresh complete media was added for 48h.

### Western-Blot:

Brains were dissected and frozen in liquid nitrogen. They were lysed in RIPA buffer (Research products, R26200-250.0) supplemented with protease and phosphatase inhibitor cocktails (Roche, 11836170001 and 4906845001) using a Tissue-Lyser (5 times 5 min at 30 shakes/second). For western blot on cell culture, cells were washed with PBS and lysed in RIPA buffer with protease and phosphatase inhibitors cocktails. All protein lysates were then centrifuged 15 min at 13200 RPM at 4° C. and supernatants were isolated. Protein concentration were quantified by BCA assay (Thermo Scientific, 23225) according to the manufacturer's instructions. 30 ug of protein were diluted in Laemmli buffer (Bio-Rad, 1610747) boiled at 95° C. for 5 min and loaded in 4-15% acrylamide gels (Bio-Rad). After electrophoresis, proteins were transferred on a polyvinylidene difluoride (PVDF) membrane and incubated in TBS 0.1% Tween supplemented with 5% BSA for 1 hour to block non-specific binding. The following antibodies were incubated overnight at 4° C.: Unc5B (Cell Signaling, 13851S), Unc5B (R&D, AF1006), Claudin5 (Invitrogen, 35-2500), PDGFRb (Cell Signaling, 3169S), GFAP (DAKO, Z0334), Zol (Invitrogen, 61-7300), JAMA (Invitrogen, 36-1700), Occludin (Invitrogen, 33-1500), Ubiquitin (Cell Signaling, 3936S), VEGFR2 Y949 (Cell Signaling, 4991S), VEGFR2 Y1173 (Cell Signaling, 2478S), Ve-cadherin (BD Pharmingen, 555289), pLRP6 (Cell Signaling, 2568S), LRP6 (Cell Signaling, 3395S), bcatenin (Cell Signaling, 8480S), LEF1 (Cell Signaling, 2230S) and Actin (Sigma, A1978). Then, membranes were washed 4×10 min in TBS 0.1% Tween and incubated with one of the following peroxidase-conjugated secondary antibodies diluted in TBS 0.1% Tween supplemented with 5% BSA for 2 hrs at room temperature: horse anti-mouse IgG(H+L) (Vector laboratories, PI-2000), goat anti-rabbit IgG(H+L) (Vector laboratories, PI-1000), goat anti-rat IgG(H+L) (Vector laboratories, PI-9400), horse anti-goat IgG(H+L) (Vector laboratories, 11 PI-9500). After 4×10 min wash, western blot bands were acquired using ECL western blotting system (Thermo Scientific, 32106) or west femto maximum sensitivity substrate (Thermo Scientific, 34095) on a Biorad Gel Doc EQ System with Universal Hood II imaging system equipped with Image Lab software.

### Immunoprecipitation:

Brain tissues were lysed using NP40 lysis buffer (Boston bioproducts, BP-119X) supplemented with protease and phosphatase inhibitor cocktails (Roche, 11836170001 and 4906845001) using a Tissue-Lyser (5×5 min at 30 shakes/second). Protein concentrations were quantified by BCA assay (Thermo Scientific, 23225) according to the manufacturer's instructions. 300 μg of protein were diluted in 1 ml of NP40 buffer containing protease and phosphatase inhibitors for each condition. In the meantime, protein A/G magnetic beads (Thermo fischer, 88802) were washed 5×10 min with NP40 buffer. Protein lysates were then incubated with 30 μl of A/G magnetic beads for 1 hour at 4° C. under gentle rotation. Once precleared, protein lysates were incubated overnight at 4° C. under gentle rotation with 10 μg of one of the following antibodies: Unc5B (cell signaling, 13851S), Unc5B (R&D, AF1006), Claudin5 (Invitrogen, 35-2500). The appropriate IgG was incubated and used as negative control (cell signaling). The next day, 40 μl of A/G magnetic beads were added to each protein lysate for 2 hour at 4° C. under gentle rotation. Beads were then isolated using magnetic separator (Invitrogen) and washed 5× with NP40 buffer. After the last wash, supernatants were removed and beads were resuspended in 40 μl of Laemmli buffer (Bio-Rad, 1610747), boiled at 95° C. for 5 min and loaded onto 4-15% gradient gels. Western blotting was performed as described elsewhere herein.

### Quantitative Real-Time PCR Analysis:

RNA was isolated using Trizol reagent (Life Technologies, 15596018) according to the manufacturer's instructions and quantified RNA concentrations using nanodrop 2000 (ThermoScientific). 300 ng of RNA were reverse transcribed into cDNA using iScript cDNA synthesis kit (Bio-rad, 170-8891). Real-time qPCR was then performed in duplicates using CFX-96 real timePCR device (Bio-rad). The following primers were obtained from Qiagen: mouse Unc5B(QT00167846), mouse Claudin5 (QT00254905), mouse LEF1 (QT00148834), mouse SLC2A112 (QT01044953), mouse PLVAP (QT00290584). Mouse GAPDH (QT01658692) was used as a housekeeping gene for all reactions.

### Immunostaining:

Brains were collected and placed in 3.7% formaldehyde overnight at 4° C. Brains were then washed 3 times 10 min with TNT buffer (for 100 ml: 10 ml Tris IM pH7.4, 3 ml NaCl 5M, 50 μl Triton X-100) and embedded in 2% agarose. 150 μm sections were prepared using a Leica VT 1000S vibratome and placed in TNTB buffer (TNT buffer supplemented with 5% donkey serum) for 24h at 4° C. Primary antibodies were diluted in TNTB and placed for 48h at 4° C. Then, sections were washed 5×30 min with TNT buffer and incubated for 24h at 4° C. with secondary antibodies diluted in TNTB buffer. After 5×30 min wash with TNT, sections were mounted using DAKO mounting medium (Agilent, S302380-2).

For whole-mount retina immunostaining, eyes were collected and fixed in 3.7% formaldehyde for 18 min. Retinas were then dissected and removed from the eyeball. Primary antibodies were diluted in blocking solution (100 mM Tris, pH 7.4, 150 mM NaCl, 0.05% Triton X-100) and incubated overnight at 4° C. Next, retinas were washed with PBS 3×10 min and incubated with secondary antibodies and Alexa Fluor-coupled Isolectin B4 in PBLEC buffer (1 mM PBS, 1 mM MgCl₂, 1 mM CaCl₂), 0.1 mM MnCl₂, 1% Triton X-100). After 3×10 min wash with PBS, retinas were mounted using DAKO mounting medium.

For brain endothelial cells immunostaining, Bend3 cells were seeded on 18 mm glass coverslips (Fischer Scientific, 12542A). At 95-100% confluence, cells were washed with PBS and fixed with 3.7% formaldehyde for 10 min. After 3×PBS wash, cells were incubated with 0).2% TritonX100 diluted in PBS for an additional 10 min, washed 3 times and incubated with blocking solution (2% BSA, 3% Donkey serum diluted in PBS) for 1 hour at room temperature. Primary antibodies were then diluted in blocking solution and incubated on coverslips overnight at 4° C. After 3×5 min washes, secondary antibodies diluted in blocking buffer were incubated on coverslips for 2 hour at room temperature. Coverslips were then washed 3×5 min with PBS and mounted using DAKO mounting medium.

The following antibodies were used: Podocalyxin (RD, AF1556), Unc5B (Cell signaling. 13851S), Unc5B (R&D, AF1006) Claudin5 (Invitrogen, 34-1600), Claudin5-GFP (Invitrogen, 352588), Occludin (Invitrogen, 33-1500), GFAP (Millipore, MAB360), Aquaporin4 (Millipore, AB3068), PDGFRb, LEF1 (Cell Signaling, 2230S), ERG1/2/3-647 (Abcam, 196149), Endomucin (Hycult biotech, HM1108), IB4-GFP (Invitrogen, 121411), fibrinogen (DAKO, A0080), DAPI (Thermo Fischer, 62248). All corresponding secondary antibodies were purchased from Invitrogen as Alexa Fluor (488, 568, 647) donkey anti-primary antibodies (H+L).

### Mouse Lung Endothelial Cell Isolation:

Mouse lung was collected at P5 or P12 and minced them into small pieces. Lungs were incubated in digestion buffer (5 ml of DMEM supplemented with 5 mg of collagenase I (Worthington LS004196), 10 μl of IM Ca²⁺ and 10 μl of IM Mg²⁺) for 1 hour at 37° C. with shaking every 10 min. Once fully lysed, lung lysates were filtered through a 40 μm cell strainer (Falcon, 352340) into a solution of 3 ml FBS. Samples were centrifuged for 10 min at 1500 RPM and pellets were resuspended in PBS 0.1% BSA. In the meantime, rat anti-mouse CD31 (BD Pharmigen, 553370)) was incubated with sheep anti-rat IgG magnetic dynabeads (Invitrogen, 11035) in a solution of sterile PBS 0.1% BSA (120 μl of beads, 24 μl of antibodies in 12 ml PBS 0.1% BSA). Solutions were place under gentle rotation at room temperature for 2 hours to allow proper coupling of antibodies and beads. Coupled beads were next isolated using a magnetic separator and incubated in the resuspended lung lysate for 30 min. After 5 washes with PBS 0.1% BSA, beads were separated using magnetic separator and seeded in 60 mm dishes containing mouse lung endothelial cell media (DMEM high glucose, 20% FBS, 1% Penicillin Streptomycin, 2% mitogen (Alta Aesar BT203). Purified endothelial cells were cultured at 37° C. and 5% CO₂ until confluence was reached, and then harvested.

### Intravenous Injection of Tracer and Antibodies:

All fluorescent tracers were injected intravenously into the lateral tail vein in adult mice (8-10 weeks old), and left to circulate for 30 min. Lysine-fixable Cadaverine conjugated to Alexa Fluor-555 (Invitrogen) was injected at a concentration of 100 μg Cadaverine/20g of mice diluted in saline. Lysine-fixable 10, 40 or 70 kDa dextran conjugated to tetramethylrhodamine (Invitrogen) were injected at a concentration of 250 μg dextran/20g of mice.

### BBB Leak Quantification:

To assess tracer leak, animals were perfused in the left ventricle with PBS. Brains (and other organs) were then collected, and their weight measured. Next, brains were incubated in formamide (Sigma-Aldrich, F7503) and incubated at 56° C. for 48 hours. Dye fluorescence was then measured using a spectrophotometer at the adequate emission and excitation wavelength. Results were normalized to the corresponding brain weight and reported to a standard made of known concentrations of dye diluted in formamide. Results are shown as ng of dye per mg of brain tissue.

### MRI:

Magnetic resonance imaging (MRI) was performed in mice under isoflurane anesthesia (2% inair) in a 4.7 T magnetic resonance scanner (Bruker BioSpec 47/40USR). Brain images were obtained using a Spin-Echo (SE) TI weighted sequence (TE/TR: 15/250 ms: matrix: 128×128: slice thickness: 1 mm: with no gap: 12 averages) in the axial and coronal planes after intravenous injections of 100 μL gadoteric acid (0.1 mmol/mL). Imaging was repeated every hour during the first 4 hours and at 24 hours after antibody injection.

### Live Imaging:

Craniotomy was performed by drilling a 5-mm circle between lambdoid, sagittal, and coronal sutures of the skull on ketamine/xylazine anesthetized ROSAmT/mG mice. After skull removal, the cortex was sealed with a glass coverslip cemented on top of the mouse skull. Live imaging was done 2 weeks later. For multiphoton excitation of endogenous fluorophores and injected dyes, a Leica SP8 DIVE in vivo imaging system was used equipped with 4tune spectral external hybrid detectors and an InSightX3 laser (SpectraPhysics). The microscope was equipped with in house designed mouse holding platform for intravital imaging (stereotactic frame, Narishige: gas anesthesia and body temperature monitoring/control, Minerve). Alexa Fluor 647 coupled 10,000 MW Dextran was acquired at 1200-nm wavelength, FITC coupled 2,000,000 MW Dextran was acquired at 950-nm wavelength and Hoechst was acquired at 800-nm wavelength. Mice were injected intravenously with 10 mg/kg of UNC5B blocking or control antibodies and 1 hour later with Dextran and/or Hoechst, followed by imaging every five minutes over 30 to 90 minutes.

### Confocal Microscopy:

Confocal images were acquired on a laser scanning fluorescence microscope (Zeiss LSM800) using the appropriate software (ZEN system). 20× and 63× oil immersion objectives were used for acquisition using selective laser excitation (405, 488, 547, or 647 nm).

### Transmission Electron Microscopy:

Adult mice were transcardiacaly perfused with PBS and 4% paraformaldehyde in PBS, followed by immersion fixation with a mixture of 2.5% glutaraldehyde and 2% paraformaldehyde in 0.1 M sodium cacodylate buffer (pH 7.4) overnight at 4° C. 150 μm-thick vibratome sections were prepared the next day. Mouse brain tissue was post-fixed in 1% OsO₄ at room temperature for one hour, specimens were then en bloc stained with 2% aqueous uranyl acetate for 30 min, dehydrated in a graded series of ethanol to 100%, substituted with propylene oxide and embedded in EMbed 812 resin. Sample blocks were polymerized in an oven at 60° C. overnight. Thin sections (60 nm) were cut by a Leica ultramicrotome (UC7) and post-stained with 2% uranyl acetate and lead citrate. Sections were examined with a FEI Tecnai transmission electron microscope at 80 kV accelerating voltage, digital images were recorded with an Olympus Morada CCD camera and iTEM imaging software.

### Statistical Analysis:

All in vivo experiments were done on littermates with similar body weight per condition and reproduced on at least 3 different litters. Statistical analysis was performed using GraphPad Prism 8 software. Mann-Whitney U test was performed for statistical analysis on two groups. ANOVA followed by Bonferroni's multiple comparisons test was performed for statistical analysis between 3 or more groups. Mantel-cox test was performed for survival curve statistical analysis.

TABLE 1
Amino acids sequences of light and heavy chains variable regions (respectively
CDR-L1 to -L3 and CDR-H1 to -H3), and light and heavy chains of the antibodies of the
disclosure.

| | SEQ | |
|---|---|---|
| | ID | |
| Name: | NO: | Sequence: |
| CDR-L1 | 1. | SVSSAVA |
| CDR-L2 | 2. | SASSLYS |
| CDR-L3-1095 | 3. | QHYSLF |
| CDR-L3-1096 | 4. | QGFAPF |
| CDR-L3-1097 | 5. | QSYGPF |
| CDR-L3-1098 | 6. | QSYSPF |
| CDR-L3-1099 | 7. | QHYALF |
| CDR-L3-1100 | 8. | QHYGLI |
| CDR-L3-1101 | 9. | QAGWPI |
| CDR-L3-1102 | 10. | QAYSPF |
| CDR-L3-1103 | 11 | QHYSYPI |
| CDR-L3-1104 | 12. | QAYALI |
| CDR-H1 | 13. | FNISYSSIHW |
| CDR-H1 | 14. | FNLYYSSIHW |
| CDR-H1 | 15. | FNLYYYSMHW |
| CDR-H1 | 16. | FNIYYSYMHW |
| CDR-H1 | 17. | FNLYYYSIHW |
| CDR-H1 | 18. | FNIYYSSMHW |
| CDR-H1 | 19. | FNISSYYSIHW |
| CDR-H2 | 20. | AYIYPYYGYTY |
| CDR-H2 | 21. | ASIYSYSSYTS |
| CDR-H2 | 22. | ASIYPYSSYTS |
| CDR-H2 | 23. | ASIYSSSGYTS |
| CDR-H2 | 24. | ASIYSSSSSTY |
| CDR-H3 | 25. | RGYGIDY |
| CDR-H3 | 26. | RSYAMDY |
| CDR-H3 | 27. | RHYAMDY |
| CDR-H3 | 28. | RGYAFNY |
| CDR-H3 | 29. | RGYAMDY |
| CDR-H3 | 30. | RGYYAGYFGIDY |
| CDR-H3 | 31. | RSFAMDY |
| CDR-H3 | 32. | RGYGLDY |
| Light Chain- | 33. | MVLQTQVFISLLLWISGAYGSDIQMTQSPSSLSASVGDRVTITC |
| 1095 | | RASQSVSSAVAWYQQKPGKAPKLLIYSASSLYSGVPSRFSGSR |
| | | SGTDFTLTISSLQPEDFATYYCQQHYSLFTFGQGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC* |
| Light Chain- | 34. | MVLQTQVFISLLLWISGAYGSDIQMTQSPSSLSASVGDRVTITC |
| 1096 | | RASQSVSSAVAWYQQKPGKAPKLLIYSASSLYSGVPSRFSGSR |
| | | SGTDFTLTISSLQPEDFATYYCQQGFAPFTFGQGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC* |
| Light Chain- | 35. | MVLQTQVFISLLLWISGAYGSDIQMTQSPSSLSASVGDRVTITC |
| 1097 | | RASQSVSSAVAWYQQKPGKAPKLLIYSASSLYSGVPSRFSGSR |
| | | SGTDFTLTISSLQPEDFATYYCQQSYGPFTFGQGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC |
| Light Chain- | 36. | MVLQTQVFISLLLWISGAYGSDIQMTQSPSSLSASVGDRVTITC |
| 1098 | | RASQSVSSAVAWYQQKPGKAPKLLIYSASSLYSGVPSRFSGSR |
| | | SGTDFTLTISSLQPEDFATYYCQQSYSPFTFGQGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC |
| Light Chain- | 37. | MVLQTQVFISLLLWISGAYGSDIQMTQSPSSLSASVGDRVTITC |
| 1099 | | RASQSVSSAVAWYQQKPGKAPKLLIYSASSLYSGVPSRFSGSR |
| | | SGTDFTLTISSLQPEDFATYYCQQHYALFTFGQGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC |
| Light Chain- | 38. | MVLQTQVFISLLLWISGAYGSDIQMTQSPSSLSASVGDRVTITC |
| 1100 | | RASQSVSSAVAWYQQKPGKAPKLLIYSASSLYSGVPSRFSGSR |
| | | SGTDFTLTISSLQPEDFATYYCQQHYGLITFGQGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC |
| Light Chain- | 39 | MVLQTQVFISLLLWISGAYGSDIQMTQSPSSLSASVGDRVTITC |
| 1101 | | RASQSVSSAVAWYQQKPGKAPKLLIYSASSLYSGVPSRFSGSR |
| | | SGTDFTLTISSLQPEDFATYYCQQAGWPITFGQGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC |
| Light Chain- | 40. | MVLQTQVFISLLLWISGAYGSDIQMTQSPSSLSASVGDRVTITC |
| 1102 | | RASQSVSSAVAWYQQKPGKAPKLLIYSASSLYSGVPSRFSGSR |
| | | SGTDFTLTISSLQPEDFATYYCQQAYSPFTFGQGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC |
| Light Chain- | 41. | MVLQTQVFISLLLWISGAYGSDIQMTQSPSSLSASVGDRVTITC |
| 1103 | | RASQSVSSAVAWYQQKPGKAPKLLIYSASSLYSGVPSRFSGSR |
| | | SGTDFTLTISSLQPEDFATYYCQQHYSYPITFGQGTKVEIKRTV |
| | | AAPSVFIFPPSDEQLKSGTASVVCLLNNFYPREAKVQWKVDN |
| | | ALQSGNSQESVTEQDSKDSTYSLSSTLTLSKADYEKHKVYAC |
| | | EVTHQGLSSPVTKSFNRGEC |
| Light Chain- | 42. | MVLQTQVFISLLLWISGAYGSDIQMTQSPSSLSASVGDRVTITC |
| 1104 | | RASQSVSSAVAWYQQKPGKAPKLLIYSASSLYSGVPSRFSGSR |
| | | SGTDFTLTISSLQPEDFATYYCQQAYALITFGQGTKVEIKRTVA |
| | | APSVFIFPPSDEQLKSGTASVVCLLNNFYPREAKVQWKVDNA |
| | | LQSGNSQESVTEQDSKDSTYSLSSTLTLSKADYEKHKVYACE |
| | | VTHQGLSSPVTKSFNRGEC |
| Heavy Chain- | 43. | MKHLWFFLLLVAAPRWVLSEISEVQLVESGGGLVQPGGSLRL |
| 1095 | | SCAASGENISYSSIHWVRQAPGKGLEWVAYIYPYYGYTYYAD |
| | | SVKGRFTISADTSKNTAYLQMNSLRAEDTAVYYCARGYGIDY |
| | | WGQGTLVTVSSASTKGPSVFPLAPCSRSTSESTAALGCLVKDY |
| | | FPEPVTVSWNSGALTSGVHTFPAVLQSSGLYSLSSVVTVPSSN |
| | | FGTQTYTCNVDHKPSNTKVDKTVERKCCVECPPCPAPPVAGP |
| | | SVFLFPPKPKDTLMISRTPEVTCVVVDVSHEDPEVQFNWYVD |
| | | GVEVHNAKTKPREEQFNSTFRVVSVLTVVHQDWLNGKEYKC |
| | | KVSNKGLPAPIEKTISKTKGQPREPQVYTLPPSREEMTKNQVS |
| | | LTCLVKGFYPSDISVEWESNGQPENNYKTTPPMLDSDGSFFLY |
| | | SKLTVDKSRWQQGNVFSCSVMHEALHNHYQKSLSLSPGK |
| Heavy Chain- | 44. | MKHLWFFLLLVAAPRWVLSEISEVQLVESGGGLVQPGGSLRL |
| 1096 | | SCAASGFNLYYSSIHWVRQAPGKGLEWVASIYSYSSYTSYAD |
| | | SVKGRFTISADTSKNTAYLQMNSLRAEDTAVYYCARSYAMD |
| | | YWGQGTLVTVSSASTKGPSVFPLAPCSRSTSESTAALGCLVKD |
| | | YFPEPVTVSWNSGALTSGVHTFPAVLQSSGLYSLSSVVTVPSS |
| | | NFGTQTYTCNVDHKPSNTKVDKTVERKCCVECPPCPAPPVAG |
| | | PSVFLFPPKPKDTLMISRTPEVTCVVVDVSHEDPEVQFNWYV |
| | | DGVEVHNAKTKPREEQFNSTFRVVSVLTVVHQDWLNGKEYK |
| | | CKVSNKGLPAPIEKTISKTKGQPREPQVYTLPPSREEMTKNQV |
| | | SLTCLVKGFYPSDISVEWESNGQPENNYKTTPPMLDSDGSFFL |
| | | YSKLTVDKSRWQQGNVFSCSVMHEALHNHYTQKSLSLSPGK |
| Heavy Chain- | 45. | MKHLWFFLLLVAAPRWVLSEISEVQLVESGGGLVQPGGSLRL |
| 1097 | | SCAASGFNLYYYSMHWVRQAPGKGLEWVASIYSYSSYTSYA |
| | | DSVKGRFTISADTSKNTAYLQMNSLRAEDTAVYYCARSYAM |
| | | DYWGQGTLVTVSSASTKGPSVFPLAPCSRSTSESTAALGCLVK |
| | | DYFPEPVTVSWNSGALTSGVHTFPAVLQSSGLYSLSSVVTVPS |
| | | SNFGTQTYTCNVDHKPSNTKVDKTVERKCCVECPPCPAPPVA |
| | | GPSVFLFPPKPKDTLMISRTPEVTCVVVDVSHEDPEVQFNWY |
| | | VDGVEVHNAKTKPREEQFNSTFRVVSVLTVVHQDWLNGKEY |
| | | KCKVSNKGLPAPIEKTISKTKGQPREPQVYTLPPSREEMTKNQ |
| | | VSLTCLVKGFYPSDISVEWESNGQPENNYKTTPPMLDSDGSFF |
| | | LYSKLTVDKSRWQQGNVFSCSVMHEALHNHYTQKSLSLSPG |
| | | K |
| Heavy Chain- | 46. | MKHLWFFLLLVAAPRWVLSEISEVQLVESGGGLVQPGGSLRL |
| 1098 | | SCAASGFNIYYSYMHWVRQAPGKGLEWVASIYSYSSYTSYA |
| | | DSVKGRFTISADTSKNTAYLQMNSLRAEDTAVYYCARHYAM |
| | | DYWGQGTLVTVSSASTKGPSVFPLAPCSRSTSESTAALGCLVK |
| | | DYFPEPVTVSWNSGALTSGVHTFPAVLQSSGLYSLSSVVTVPS |
| | | SNFGTQTYTCNVDHKPSNTKVDKTVERKCCVECPPCPAPPVA |
| | | GPSVFLFPPKPKDTLMISRTPEVTCVVVDVSHEDPEVQFNWY |
| | | VDGVEVHNAKTKPREEQFNSTFRVVSVLTVVHQDWLNGKEY |
| | | KCKVSNKGLPAPIEKTISKTKGQPREPQVYTLPPSREEMTKNQ |
| | | VSLTCLVKGFYPSDISVEWESNGQPENNYKTTPPMLDSDGSFF |
| | | LYSKLTVDKSRWQQGNVFSCSVMHEALHNHYTQKSLSLSPG |
| | | K |
| Heavy Chain- | 47. | MKHLWFFLLLVAAPRWVLSEISEVQLVESGGGLVQPGGSLRL |
| 1099 | | SCAASGFNLYYYSIHWVRQAPGKGLEWVASIYPYSSYTSYAD |
| | | SVKGRFTISADTSKNTAYLQMNSLRAEDTAVYYCARGYAFN |
| | | YWGQGTLVTVSSASTKGPSVFPLAPCSRSTSESTAALGCLVKD |
| | | YFPEPVTVSWNSGALTSGVHTFPAVLQSSGLYSLSSVVTVPSS |
| | | NFGTQTYTCNVDHKPSNTKVDKTVERKCCVECPPCPAPPVAG |
| | | PSVFLFPPKPKDTLMISRTPEVTCVVVDVSHEDPEVQFNWYV |
| | | DGVEVHNAKTKPREEQFNSTFRVVSVLTVVHQDWLNGKEYK |
| | | CKVSNKGLPAPIEKTISKTKGQPREPQVYTLPPSREEMTKNQV |
| | | SLTCLVKGFYPSDISVEWESNGQPENNYKTTPPMLDSDGSFFL |
| | | YSKLTVDKSRWQQGNVFSCSVMHEALHNHYTQKSLSLSPGK |
| Heavy Chain- | 48. | MKHLWFFLLLVAAPRWVLSEISEVQLVESGGGLVQPGGSLRL |
| 1100 | | SCAASGFNLYYSSIHWVRQAPGKGLEWVASIYSYSSYTSYAD |
| | | SVKGRFTISADTSKNTAYLQMNSLRAEDTAVYYCARGYAMD |
| | | YWGQGTLVTVSSASTKGPSVFPLAPCSRSTSESTAALGCLVKD |
| | | YFPEPVTVSWNSGALTSGVHTFPAVLQSSGLYSLSSVVTVPSS |
| | | NFGTQTYTCNVDHKPSNTKVDKTVERKCCVECPPCPAPPVAG |
| | | PSVFLFPPKPKDTLMISRTPEVTCVVVDVSHEDPEVQFNWYV |
| | | DGVEVHNAKTKPREEQFNSTFRVVSVLTVVHQDWLNGKEYK |
| | | CKVSNKGLPAPIEKTISKTKGQPREPQVYTLPPSREEMTKNQV |
| | | SLTCLVKGFYPSDISVEWESNGQPENNYKTTPPMLDSDGSFFL |
| | | YSKLTVDKSRWQQGNVFSCSVMHEALHNHYTQKSLSLSPGK |
| Heavy Chain- | 49. | MKHLWFFLLLVAAPRWVLSEISEVQLVESGGGLVQPGGSLRL |
| 1101 | | SCAASGFNIYYSSMHWVRQAPGKGLEWVAYISSSYGYTYYA |
| | | DSVKGRFTISADTSKNTAYLQMNSLRAEDTAVYYCARGYYA |
| | | GYFGIDYWGQGTLVTVSSASTKGPSVFPLAPCSRSTSESTAAL |
| | | GCLVKDYFPEPVTVSWNSGALTSGVHTFPAVLQSSGLYSLSS |
| | | VVTVPSSNFGTQTYTCNVDHKPSNTKVDKTVERKCCVECPPC |
| | | PAPPVAGPSVFLFPPKPKDTLMISRTPEVTCVVVDVSHEDPEV |
| | | QFNWYVDGVEVHNAKTKPREEQFNSTFRVVSVLTVVHQDW |
| | | LNGKEYKCKVSNKGLPAPIEKTISKTKGQPREPQVYTLPPSRE |
| | | EMTKNQVSLTCLVKGFYPSDISVEWESNGQPENNYKTTPPML |
| | | DSDGSFFLYSKLTVDKSRWQQGNVFSCSVMHEALHNHYTQK |
| | | SLSLSPGK |
| Heavy Chain- | 50. | MKHLWFFLLLVAAPRWVLSEISEVQLVESGGGLVQPGGSLRL |
| 1102 | | SCAASGFNIYYSSMHWVRQAPGKGLEWVASIYSYSSYTSYAD |
| | | SVKGRFTISADTSKNTAYLQMNSLRAEDTAVYYCARSFAMD |
| | | YWGQGTLVTVSSASTKGPSVFPLAPCSRSTSESTAALGCLVKD |
| | | YFPEPVTVSWNSGALTSGVHTFPAVLQSSGLYSLSSVVTVPSS |
| | | NFGTQTYTCNVDHKPSNTKVDKTVERKCCVECPPCPAPPVAG |
| | | PSVFLFPPKPKDTLMISRTPEVTCVVVDVSHEDPEVQFNWYV |
| | | DGVEVHNAKTKPREEQFNSTFRVVSVLTVVHQDWLNGKEYK |
| | | CKVSNKGLPAPIEKTISKTKGQPREPQVYTLPPSREEMTKNQV |
| | | SLTCLVKGFYPSDISVEWESNGQPENNYKTTPPMLDSDGSFFL |
| | | YSKLTVDKSRWQQGNVFSCSVMHEALHNHYTQKSLSLSPGK |
| Heavy Chain- | 51. | MKHLWFFLLLVAAPRWVLSEISEVQLVESGGGLVQPGGSLRL |
| 1103 | | SCAASGFNISYYSIHWVRQAPGKGLEWVASIYSSSGYTSYADS |
| | | VKGRFTISADTSKNTAYLQMNSLRAEDTAVYYCARGYGLDY |
| | | WGQGTLVTVSSASTKGPSVFPLAPCSRSTSESTAALGCLVKDY |
| | | FPEPVTVSWNSGALTSGVHTFPAVLQSSGLYSLSSVVTVPSSN |
| | | FGTQTYTCNVDHKPSNTKVDKTVERKCCVECPPCPAPPVAGP |
| | | SVFLFPPKPKDTLMISRTPEVTCVVVDVSHEDPEVQFNWYVD |
| | | GVEVHNAKTKPREEQFNSTFRVVSVLTVVHQDWLNGKEYKC |
| | | KVSNKGLPAPIEKTISKTKGQPREPQVYTLPPSREEMTKNQVS |
| | | LTCLVKGFYPSDISVEWESNGQPENNYKTTPPMLDSDGSFFLY |
| | | SKLTVDKSRWQQGNVFSCSVMHEALHNHYTQKSLSLSPGK |
| Heavy Chain- | 52. | MKHLWFFLLLVAAPRWVLSEISEVQLVESGGGLVQPGGSLRL |
| 1104 | | SCAASGFNISYYSIHWVRQAPGKGLEWVASIYSSSSSTYYADS |
| | | VKGRFTISADTSKNTAYLQMNSLRAEDTAVYYCARGYGLDY |
| | | WGQGTLVTVSSASTKGPSVFPLAPCSRSTSESTAALGCLVKDY |
| | | FPEPVTVSWNSGALTSGVHTFPAVLQSSGLYSLSSVVTVPSSN |
| | | FGTQTYTCNVDHKPSNTKVDKTVERKCCVECPPCPAPPVAGP |
| | | SVFLFPPKPKDTLMISRTPEVTCVVVDVSHEDPEVQFNWYVD |
| | | GVEVHNAKTKPREEQFNSTFRVVSVLTVVHQDWLNGKEYKC |
| | | KVSNKGLPAPIEKTISKTKGQPREPQVYTLPPSREEMTKNQVS |
| | | LTCLVKGFYPSDISVEWESNGQPENNYKTTPPMLDSDGSFFLY |
| | | SKLTVDKSRWQQGNVFSCSVMHEALHNHYTQKSLSLSPGK |
| Heavy Chain- | 53. | ATGAAGCACCTGTGGTTCTTTCTGCTGCTGGTGGCCGCTCC |
| 1095 | | AAGATGGGTGCTGAGCGAGATCTCTGAGGTGCAGCTGGTG |
| | | GAGAGCGGAGGAGGACTGGTGCAGCCAGGAGGATCCCTGA |
| | | GACTGAGCTGCGCCGCTTCTGGCTTCAACATCTCTTACTCC |
| | | AGCATCCATTGGGTGCGCCAGGCTCCAGGCAAGGGACTGG |
| | | AGTGGGTGGCTTACATCTATCCTTACTATGGCTACACCTAC |
| | | TATGCCGACTCTGTGAAGGGCAGGTTCACCATCTCCGCCGA |
| | | TACCAGCAAGAACACAGCTTATCTGCAGATGAACTCCCTG |
| | | AGGGCCGAGGACACCGCCGTGTACTATTGTGCTCGGGGCT |
| | | ACGGCATCGATTATTGGGGCCAGGGCACCCTGGTGACAGT |
| | | GTCTTCCGCTAGCACCAAGGGCCCCTCCGTGTTCCCTCTGG |
| | | CCCCCTGTAGCAGAAGCACAAGCGAGTCCACCGCCGCCCT |
| | | GGGCTGTCTGGTGAAGGACTACTTTCCTGAGCCTGTGACCG |
| | | TGTCCTGGAATAGCGGCGCCCTGACCTCCGGCGTGCACACA |
| | | TTTCCTGCCGTGCTGCAGTCCTCCGGCCTGTACAGCCTGAG |
| | | CAGCGTGGTGACAGTGCCCAGCTCCAACTTCGGCACACAG |
| | | ACATACACCTGCAATGTGGATCACAAGCCTTCCAACACAA |
| | | AGGTGGACAAGACCGTGGAGAGAAAGTGTTGCGTGGAGTG |
| | | CCCTCCTTGCCCCGCCCCTCCAGTGGCTGGACCATCCGTGT |
| | | TCCTGTTTCCCCCTAAGCCCAAGGATACACTGATGATCAGC |
| | | AGGACCCCCGAGGTGACATGCGTGGTGGTGGATGTGAGCC |
| | | ACGAGGACCCCGAGGTGCAGTTTAACTGGTACGTGGACGG |
| | | CGTGGAGGTGCACAACGCCAAGACAAAGCCCAGGGAGGA |
| | | GCAGTTCAATTCCACCTTCAGAGTGGTGAGCGTGCTGACCG |
| | | TGGTGCACCAGGATTGGCTGAATGGCAAGGAGTACAAGTG |
| | | TAAGGTGTCCAACAAGGGCCTGCCCGCCCCCATCGAGAAG |
| | | ACCATCTCCAAGACAAAGGGCCAGCCCAGGGAGCCCCAGG |
| | | TGTACACACTGCCCCCTAGCAGGGAGGAGATGACAAAGAA |
| | | CCAGGTGTCCCTGACCTGCCTGGTGAAGGGCTTCTACCCTA |
| | | GCGACATCAGCGTGGAGTGGGAGTCCAATGGCCAGCCCGA |
| | | GAATAACTACAAGACCACCCCCCCTATGCTGGATAGCGAC |
| | | GGCAGCTTTTTCCTGTACTCCAAGCTGACAGTGGACAAGTC |
| | | CAGGTGGCAGCAGGGCAACGTGTTTAGCTGCAGCGTGATG |
| | | CACGAGGCCCTGCACAACCACTACACCCAGAAGAGCCTGT |
| | | CCCTGTCCCCCGGCAAGTGAGCGGCCGC |
| Heavy Chain- | 54. | ATGAAGCACCTGTGGTTCTTTCTGCTGCTGGTGGCTGCTCC |
| 1096 | | AAGATGGGTGCTGTCCGAGATCAGCGAGGTGCAGCTGGTG |
| | | GAGAGCGGAGGAGGACTGGTGCAGCCTGGAGGCAGCCTGA |
| | | GACTGTCTTGCGCCGCTTCCGGCTTCAACCTGTACTATTCC |
| | | AGCATCCATTGGGTGCGCCAGGCTCCAGGCAAGGGACTGG |
| | | AGTGGGTGGCTTCTATCTACTCCTATTCTTCCTACACCAGCT |
| | | ATGCCGACTCTGTGAAGGGCAGGTTCACCATCTCCGCCGAT |
| | | ACCAGCAAGAACACAGCTTACCTGCAGATGAACTCCCTGA |
| | | GGGCCGAGGACACCGCCGTGTACTATTGTGCCCGGTCCTAC |
| | | GCTATGGATTATTGGGGCCAGGGCACCCTGGTGACAGTGA |
| | | GCTCTGCTAGCACCAAGGGCCCCTCCGTGTTCCCTCTGGCC |
| | | CCCTGTAGCAGAAGCACAAGCGAGTCCACCGCCGCCCTGG |
| | | GCTGTCTGGTGAAGGACTACTTTCCTGAGCCTGTGACCGTG |
| | | TCCTGGAATAGCGGCGCCCTGACCTCCGGCGTGCACACATT |
| | | TCCTGCCGTGCTGCAGTCCTCCGGCCTGTACAGCCTGAGCA |
| | | GCGTGGTGACAGTGCCCAGCTCCAACTTCGGCACACAGAC |
| | | ATACACCTGCAATGTGGATCACAAGCCTTCCAACACAAAG |
| | | GTGGACAAGACCGTGGAGAGAAAGTGTTGCGTGGAGTGCC |
| | | CTCCTTGCCCCGCCCCTCCAGTGGCTGGACCATCCGTGTTC |
| | | CTGTTTCCCCCTAAGCCCAAGGATACACTGATGATCAGCAG |
| | | GACCCCCGAGGTGACATGCGTGGTGGTGGATGTGAGCCAC |
| | | GAGGACCCCGAGGTGCAGTTTAACTGGTACGTGGACGGCG |
| | | TGGAGGTGCACAACGCCAAGACAAAGCCCAGGGAGGAGC |
| | | AGTTCAATTCCACCTTCAGAGTGGTGAGCGTGCTGACCGTG |
| | | GTGCACCAGGATTGGCTGAATGGCAAGGAGTACAAGTGTA |
| | | AGGTGTCCAACAAGGGCCTGCCCGCCCCCATCGAGAAGAC |
| | | CATCTCCAAGACAAAGGGCCAGCCCAGGGAGCCCCAGGTG |
| | | TACACACTGCCCCCTAGCAGGGAGGAGATGACAAAGAACC |
| | | AGGTGTCCCTGACCTGCCTGGTGAAGGGCTTCTACCCTAGC |
| | | GACATCAGCGTGGAGTGGGAGTCCAATGGCCAGCCCGAGA |
| | | ATAACTACAAGACCACCCCCCCTATGCTGGATAGCGACGG |
| | | CAGCTTTTTCCTGTACTCCAAGCTGACAGTGGACAAGTCCA |
| | | GGTGGCAGCAGGGCAACGTGTTTAGCTGCAGCGTGATGCA |
| | | CGAGGCCCTGCACAACCACTACACCCAGAAGAGCCTGTCC |
| | | CTGTCCCCCGGCAAGTGAGCGGCCGC |
| Heavy Chain- | 55. | ATGAAGCACCTGTGGTTCTTTCTGCTGCTGGTGGCTGCTCC |
| 1097 | | AAGATGGGTGCTGAGCGAGATCTCTGAGGTGCAGCTGGTG |
| | | GAGTCTGGAGGAGGACTGGTGCAGCCTGGAGGATCTCTGA |
| | | GACTGTCCTGCGCTGCCTCCGGCTTCAACCTGTACTATTAC |
| | | TCCATGCATTGGGTGCGCCAGGCTCCAGGCAAGGGACTGG |
| | | AGTGGGTGGCTTCCATCTATAGCTACTCCAGCTATACCTCT |
| | | TACGCCGACTCCGTGAAGGGCAGGTTCACCATCAGCGCCG |
| | | ATACCTCTAAGAACACAGCTTATCTGCAGATGAACTCCCTG |
| | | AGGGCCGAGGACACCGCCGTGTACTACTGTGCCCGGAGCT |
| | | ATGCTATGGATTACTGGGGCCAGGGCACCCTGGTGACAGT |
| | | GTCTTCCGCTAGCACCAAGGGCCCCTCCGTGTTCCCTCTGG |
| | | CCCCCTGTAGCAGAAGCACAAGCGAGTCCACCGCCGCCCT |
| | | GGGCTGTCTGGTGAAGGACTACTTTCCTGAGCCTGTGACCG |
| | | TGTCCTGGAATAGCGGCGCCCTGACCTCCGGCGTGCACACA |
| | | TTTCCTGCCGTGCTGCAGTCCTCCGGCCTGTACAGCCTGAG |
| | | CAGCGTGGTGACAGTGCCCAGCTCCAACTTCGGCACACAG |
| | | ACATACACCTGCAATGTGGATCACAAGCCTTCCAACACAA |
| | | AGGTGGACAAGACCGTGGAGAGAAAGTGTTGCGTGGAGTG |
| | | CCCTCCTTGCCCCGCCCCTCCAGTGGCTGGACCATCCGTGT |
| | | TCCTGTTTCCCCCTAAGCCCAAGGATACACTGATGATCAGC |
| | | AGGACCCCCGAGGTGACATGCGTGGTGGTGGATGTGAGCC |
| | | ACGAGGACCCCGAGGTGCAGTTTAACTGGTACGTGGACGG |
| | | CGTGGAGGTGCACAACGCCAAGACAAAGCCCAGGGAGGA |
| | | GCAGTTCAATTCCACCTTCAGAGTGGTGAGCGTGCTGACCG |
| | | TGGTGCACCAGGATTGGCTGAATGGCAAGGAGTACAAGTG |
| | | TAAGGTGTCCAACAAGGGCCTGCCCGCCCCCATCGAGAAG |
| | | ACCATCTCCAAGACAAAGGGCCAGCCCAGGGAGCCCCAGG |
| | | TGTACACACTGCCCCCTAGCAGGGAGGAGATGACAAAGAA |
| | | CCAGGTGTCCCTGACCTGCCTGGTGAAGGGCTTCTACCCTA |
| | | GCGACATCAGCGTGGAGTGGGAGTCCAATGGCCAGCCCGA |
| | | GAATAACTACAAGACCACCCCCCCTATGCTGGATAGCGAC |
| | | GGCAGCTTTTTCCTGTACTCCAAGCTGACAGTGGACAAGTC |
| | | CAGGTGGCAGCAGGGCAACGTGTTTAGCTGCAGCGTGATG |
| | | CACGAGGCCCTGCACAACCACTACACCCAGAAGAGCCTGT |
| | | CCCTGTCCCCCGGCAAGTGAGCGGCCGC |
| Heavy Chain- | 56. | ATGAAGCACCTGTGGTTCTTTCTGCTGCTGGTGGCTGCTCC |
| 1098 | | AAGATGGGTGCTGAGCGAGATCTCTGAGGTGCAGCTGGTG |
| | | GAGTCCGGAGGAGGACTGGTGCAGCCTGGAGGATCTCTGA |
| | | GACTGTCCTGCGCTGCCTCCGGCTTCAACATCTACTATAGC |
| | | TACATGCATTGGGTGCGCCAGGCTCCAGGCAAGGGACTGG |
| | | AGTGGGTGGCTTCCATCTACAGCTATTCCAGCTACACCTCT |
| | | TATGCCGACTCCGTGAAGGGCAGGTTCACCATCAGCGCCG |
| | | ATACCTCTAAGAACACAGCTTATCTGCAGATGAACTCCCTG |
| | | AGGGCCGAGGACACCGCCGTGTACTATTGTGCCCGGCACT |
| | | ACGCTATGGATTATTGGGGCCAGGGCACCCTGGTGACAGT |
| | | GTCTTCCGCTAGCACCAAGGGCCCCTCCGTGTTCCCTCTGG |
| | | CCCCCTGTAGCAGAAGCACAAGCGAGTCCACCGCCGCCCT |
| | | GGGCTGTCTGGTGAAGGACTACTTTCCTGAGCCTGTGACCG |
| | | TGTCCTGGAATAGCGGCGCCCTGACCTCCGGCGTGCACACA |
| | | TTTCCTGCCGTGCTGCAGTCCTCCGGCCTGTACAGCCTGAG |
| | | CAGCGTGGTGACAGTGCCCAGCTCCAACTTCGGCACACAG |
| | | ACATACACCTGCAATGTGGATCACAAGCCTTCCAACACAA |
| | | AGGTGGACAAGACCGTGGAGAGAAAGTGTTGCGTGGAGTG |
| | | CCCTCCTTGCCCCGCCCCTCCAGTGGCTGGACCATCCGTGT |
| | | TCCTGTTTCCCCCTAAGCCCAAGGATACACTGATGATCAGC |
| | | AGGACCCCCGAGGTGACATGCGTGGTGGTGGATGTGAGCC |
| | | ACGAGGACCCCGAGGTGCAGTTTAACTGGTACGTGGACGG |
| | | CGTGGAGGTGCACAACGCCAAGACAAAGCCCAGGGAGGA |
| | | GCAGTTCAATTCCACCTTCAGAGTGGTGAGCGTGCTGACCG |
| | | TGGTGCACCAGGATTGGCTGAATGGCAAGGAGTACAAGTG |
| | | TAAGGTGTCCAACAAGGGCCTGCCCGCCCCCATCGAGAAG |
| | | ACCATCTCCAAGACAAAGGGCCAGCCCAGGGAGCCCCAGG |
| | | TGTACACACTGCCCCCTAGCAGGGAGGAGATGACAAAGAA |
| | | CCAGGTGTCCCTGACCTGCCTGGTGAAGGGCTTCTACCCTA |
| | | GCGACATCAGCGTGGAGTGGGAGTCCAATGGCCAGCCCGA |
| | | GAATAACTACAAGACCACCCCCCCTATGCTGGATAGCGAC |
| | | GGCAGCTTTTTCCTGTACTCCAAGCTGACAGTGGACAAGTC |
| | | CAGGTGGCAGCAGGGCAACGTGTTTAGCTGCAGCGTGATG |
| | | CACGAGGCCCTGCACAACCACTACACCCAGAAGAGCCTGT |
| | | CCCTGTCCCCCGGCAAGTGAGCGGCCGC |
| Heavy Chain- | 57. | ATGAAGCACCTGTGGTTCTTTCTGCTGCTGGTGGCCGCTCC |
| 1099 | | AAGATGGGTGCTGAGCGAGATCTCTGAGGTGCAGCTGGTG |
| | | GAGTCCGGAGGAGGACTGGTGCAGCCAGGAGGATCCCTGA |
| | | GACTGAGCTGCGCCGCTTCTGGCTTCAACCTGTACTATTAC |
| | | AGCATCCATTGGGTGCGCCAGGCTCCAGGCAAGGGACTGG |
| | | AGTGGGTGGCTTCTATCTATCCTTACTCCAGCTATACCTCCT |
| | | ACGCCGACAGCGTGAAGGGCAGATTCACAATCTCTGCCGA |
| | | TACCTCCAAGAACACAGCTTATCTGCAGATGAACTCCCTGA |
| | | GGGCCGAGGACACCGCCGTGTACTACTGTGCCCGGGGCTA |
| | | TGCTTTTAATTACTGGGGCCAGGGCACCCTGGTGACAGTGT |
| | | CTTCCGCTAGCACCAAGGGCCCCTCCGTGTTCCCTCTGGCC |
| | | CCCTGTAGCAGAAGCACAAGCGAGTCCACCGCCGCCCTGG |
| | | GCTGTCTGGTGAAGGACTACTTTCCTGAGCCTGTGACCGTG |
| | | TCCTGGAATAGCGGCGCCCTGACCTCCGGCGTGCACACATT |
| | | TCCTGCCGTGCTGCAGTCCTCCGGCCTGTACAGCCTGAGCA |
| | | GCGTGGTGACAGTGCCCAGCTCCAACTTCGGCACACAGAC |
| | | ATACACCTGCAATGTGGATCACAAGCCTTCCAACACAAAG |
| | | GTGGACAAGACCGTGGAGAGAAAGTGTTGCGTGGAGTGCC |
| | | CTCCTTGCCCCGCCCCTCCAGTGGCTGGACCATCCGTGTTC |
| | | CTGTTTCCCCCTAAGCCCAAGGATACACTGATGATCAGCAG |
| | | GACCCCCGAGGTGACATGCGTGGTGGTGGATGTGAGCCAC |
| | | GAGGACCCCGAGGTGCAGTTTAACTGGTACGTGGACGGCG |
| | | TGGAGGTGCACAACGCCAAGACAAAGCCCAGGGAGGAGC |
| | | AGTTCAATTCCACCTTCAGAGTGGTGAGCGTGCTGACCGTG |
| | | GTGCACCAGGATTGGCTGAATGGCAAGGAGTACAAGTGTA |
| | | AGGTGTCCAACAAGGGCCTGCCCGCCCCCATCGAGAAGAC |
| | | CATCTCCAAGACAAAGGGCCAGCCCAGGGAGCCCCAGGTG |
| | | TACACACTGCCCCCTAGCAGGGAGGAGATGACAAAGAACC |
| | | AGGTGTCCCTGACCTGCCTGGTGAAGGGCTTCTACCCTAGC |
| | | GACATCAGCGTGGAGTGGGAGTCCAATGGCCAGCCCGAGA |
| | | ATAACTACAAGACCACCCCCCCTATGCTGGATAGCGACGG |
| | | CAGCTTTTTCCTGTACTCCAAGCTGACAGTGGACAAGTCCA |
| | | GGTGGCAGCAGGGCAACGTGTTTAGCTGCAGCGTGATGCA |
| | | CGAGGCCCTGCACAACCACTACACCCAGAAGAGCCTGTCC |
| | | CTGTCCCCCGGCAAGTGAGCGGCCGC |
| Heavy Chain- | 58. | ATGAAGCACCTGTGGTTCTTTCTGCTGCTGGTGGCTGCTCC |
| 1100 | | AAGATGGGTGCTGTCCGAGATCAGCGAGGTGCAGCTGGTG |
| | | GAGTCTGGAGGAGGACTGGTGCAGCCTGGAGGAAGCCTGA |
| | | GACTGTCTTGCGCCGCTTCCGGCTTCAACCTGTACTATTCC |
| | | AGCATCCATTGGGTGCGCCAGGCTCCAGGCAAGGGACTGG |
| | | AGTGGGTGGCTTCTATCTACTCCTATTCTTCCTACACCAGCT |
| | | ATGCCGACTCTGTGAAGGGCAGGTTCACCATCTCCGCCGAT |
| | | ACCAGCAAGAACACAGCTTACCTGCAGATGAACTCCCTGA |
| | | GGGCCGAGGACACCGCCGTGTACTATTGTGCCCGGGGCTA |
| | | CGCTATGGATTATTGGGGCCAGGGCACCCTGGTGACAGTG |
| | | AGCTCTGCTAGCACCAAGGGCCCCTCCGTGTTCCCTCTGGC |
| | | CCCCTGTAGCAGAAGCACAAGCGAGTCCACCGCCGCCCTG |
| | | GGCTGTCTGGTGAAGGACTACTTTCCTGAGCCTGTGACCGT |
| | | GTCCTGGAATAGCGGCGCCCTGACCTCCGGCGTGCACACAT |
| | | TTCCTGCCGTGCTGCAGTCCTCCGGCCTGTACAGCCTGAGC |
| | | AGCGTGGTGACAGTGCCCAGCTCCAACTTCGGCACACAGA |
| | | CATACACCTGCAATGTGGATCACAAGCCTTCCAACACAAA |
| | | GGTGGACAAGACCGTGGAGAGAAAGTGTTGCGTGGAGTGC |
| | | CCTCCTTGCCCCGCCCCTCCAGTGGCTGGACCATCCGTGTT |
| | | CCTGTTTCCCCCTAAGCCCAAGGATACACTGATGATCAGCA |
| | | GGACCCCCGAGGTGACATGCGTGGTGGTGGATGTGAGCCA |
| | | CGAGGACCCCGAGGTGCAGTTTAACTGGTACGTGGACGGC |
| | | GTGGAGGTGCACAACGCCAAGACAAAGCCCAGGGAGGAG |
| | | CAGTTCAATTCCACCTTCAGAGTGGTGAGCGTGCTGACCGT |
| | | GGTGCACCAGGATTGGCTGAATGGCAAGGAGTACAAGTGT |
| | | AAGGTGTCCAACAAGGGCCTGCCCGCCCCCATCGAGAAGA |
| | | CCATCTCCAAGACAAAGGGCCAGCCCAGGGAGCCCCAGGT |
| | | GTACACACTGCCCCCTAGCAGGGAGGAGATGACAAAGAAC |
| | | CAGGTGTCCCTGACCTGCCTGGTGAAGGGCTTCTACCCTAG |
| | | CGACATCAGCGTGGAGTGGGAGTCCAATGGCCAGCCCGAG |
| | | AATAACTACAAGACCACCCCCCCTATGCTGGATAGCGACG |
| | | GCAGCTTTTTCCTGTACTCCAAGCTGACAGTGGACAAGTCC |
| | | AGGTGGCAGCAGGGCAACGTGTTTAGCTGCAGCGTGATGC |
| | | ACGAGGCCCTGCACAACCACTACACCCAGAAGAGCCTGTC |
| | | CCTGTCCCCCGGCAAGTGAGCGGCCGC |
| Heavy Chain- | 59. | ATGAAGCACCTGTGGTTCTTTCTGCTGCTGGTGGCTGCTCC |
| 1101 | | AAGATGGGTGCTGAGCGAGATCTCTGAGGTGCAGCTGGTG |
| | | GAGTCCGGAGGAGGACTGGTGCAGCCTGGAGGATCCCTGA |
| | | GACTGAGCTGCGCCGCTTCTGGCTTCAACATCTACTATTCC |
| | | AGCATGCATTGGGTGCGCCAGGCTCCAGGCAAGGGACTGG |
| | | AGTGGGTGGCTTACATCTCTTCCAGCTACGGCTATACCTAC |
| | | TATGCCGACTCTGTGAAGGGCAGATTCACAATCTCCGCCGA |
| | | TACCAGCAAGAACACAGCTTATCTGCAGATGAACTCCCTG |
| | | AGGGCCGAGGACACCGCCGTGTACTATTGTGCCCGGGGCT |
| | | ACTATGCTGGCTACTTTGGCATCGATTATTGGGGCCAGGGC |
| | | ACCCTGGTGACAGTGTCTTCCGCTAGCACCAAGGGCCCCTC |
| | | CGTGTTCCCTCTGGCCCCCTGTAGCAGAAGCACAAGCGAGT |
| | | CCACCGCCGCCCTGGGCTGTCTGGTGAAGGACTACTTTCCT |
| | | GAGCCTGTGACCGTGTCCTGGAATAGCGGCGCCCTGACCTC |
| | | CGGCGTGCACACATTTCCTGCCGTGCTGCAGTCCTCCGGCC |
| | | TGTACAGCCTGAGCAGCGTGGTGACAGTGCCCAGCTCCAA |
| | | CTTCGGCACACAGACATACACCTGCAATGTGGATCACAAG |
| | | CCTTCCAACACAAAGGTGGACAAGACCGTGGAGAGAAAGT |
| | | GTTGCGTGGAGTGCCCTCCTTGCCCCGCCCCTCCAGTGGCT |
| | | GGACCATCCGTGTTCCTGTTTCCCCCTAAGCCCAAGGATAC |
| | | ACTGATGATCAGCAGGACCCCCGAGGTGACATGCGTGGTG |
| | | GTGGATGTGAGCCACGAGGACCCCGAGGTGCAGTTTAACT |
| | | GGTACGTGGACGGCGTGGAGGTGCACAACGCCAAGACAAA |
| | | GCCCAGGGAGGAGCAGTTCAATTCCACCTTCAGAGTGGTG |
| | | AGCGTGCTGACCGTGGTGCACCAGGATTGGCTGAATGGCA |
| | | AGGAGTACAAGTGTAAGGTGTCCAACAAGGGCCTGCCCGC |
| | | CCCCATCGAGAAGACCATCTCCAAGACAAAGGGCCAGCCC |
| | | AGGGAGCCCCAGGTGTACACACTGCCCCCTAGCAGGGAGG |
| | | AGATGACAAAGAACCAGGTGTCCCTGACCTGCCTGGTGAA |
| | | GGGCTTCTACCCTAGCGACATCAGCGTGGAGTGGGAGTCC |
| | | AATGGCCAGCCCGAGAATAACTACAAGACCACCCCCCCTA |
| | | TGCTGGATAGCGACGGCAGCTTTTTCCTGTACTCCAAGCTG |
| | | ACAGTGGACAAGTCCAGGTGGCAGCAGGGCAACGTGTTTA |
| | | GCTGCAGCGTGATGCACGAGGCCCTGCACAACCACTACAC |
| | | CCAGAAGAGCCTGTCCCTGTCCCCCGGCAAGTGAGCGGCC |
| | | GC |
| Heavy Chain- | 60. | ATGAAGCACCTGTGGTTCTTTCTGCTGCTGGTGGCTGCTCC |
| 1102 | | AAGATGGGTGCTGTCCGAGATCAGCGAGGTGCAGCTGGTG |
| | | GAGAGCGGAGGAGGACTGGTGCAGCCTGGAGGCAGCCTGA |
| | | GACTGTCTTGCGCCGCTTCCGGCTTCAACATCTACTATTCC |
| | | AGCATGCATTGGGTGCGCCAGGCTCCAGGCAAGGGACTGG |
| | | AGTGGGTGGCTTCTATCTACTCCTATTCTTCCTACACCAGCT |
| | | ATGCCGACTCTGTGAAGGGCAGATTCACAATCTCCGCCGAT |
| | | ACCAGCAAGAACACAGCTTACCTGCAGATGAACTCCCTGA |
| | | GGGCCGAGGACACCGCCGTGTACTATTGTGCCCGGTCCTTT |
| | | GCTATGGATTATTGGGGCCAGGGCACCCTGGTGACAGTGA |
| | | GCTCTGCTAGCACCAAGGGCCCCTCCGTGTTCCCTCTGGCC |
| | | CCCTGTAGCAGAAGCACAAGCGAGTCCACCGCCGCCCTGG |
| | | GCTGTCTGGTGAAGGACTACTTTCCTGAGCCTGTGACCGTG |
| | | TCCTGGAATAGCGGCGCCCTGACCTCCGGCGTGCACACATT |
| | | TCCTGCCGTGCTGCAGTCCTCCGGCCTGTACAGCCTGAGCA |
| | | GCGTGGTGACAGTGCCCAGCTCCAACTTCGGCACACAGAC |
| | | ATACACCTGCAATGTGGATCACAAGCCTTCCAACACAAAG |
| | | GTGGACAAGACCGTGGAGAGAAAGTGTTGCGTGGAGTGCC |
| | | CTCCTTGCCCCGCCCCTCCAGTGGCTGGACCATCCGTGTTC |
| | | CTGTTTCCCCCTAAGCCCAAGGATACACTGATGATCAGCAG |
| | | GACCCCCGAGGTGACATGCGTGGTGGTGGATGTGAGCCAC |
| | | GAGGACCCCGAGGTGCAGTTTAACTGGTACGTGGACGGCG |
| | | TGGAGGTGCACAACGCCAAGACAAAGCCCAGGGAGGAGC |
| | | AGTTCAATTCCACCTTCAGAGTGGTGAGCGTGCTGACCGTG |
| | | GTGCACCAGGATTGGCTGAATGGCAAGGAGTACAAGTGTA |
| | | AGGTGTCCAACAAGGGCCTGCCCGCCCCCATCGAGAAGAC |
| | | CATCTCCAAGACAAAGGGCCAGCCCAGGGAGCCCCAGGTG |
| | | TACACACTGCCCCCTAGCAGGGAGGAGATGACAAAGAACC |
| | | AGGTGTCCCTGACCTGCCTGGTGAAGGGCTTCTACCCTAGC |
| | | GACATCAGCGTGGAGTGGGAGTCCAATGGCCAGCCCGAGA |
| | | ATAACTACAAGACCACCCCCCCTATGCTGGATAGCGACGG |
| | | CAGCTTTTTCCTGTACTCCAAGCTGACAGTGGACAAGTCCA |
| | | GGTGGCAGCAGGGCAACGTGTTTAGCTGCAGCGTGATGCA |
| | | CGAGGCCCTGCACAACCACTACACCCAGAAGAGCCTGTCC |
| | | CTGTCCCCCGGCAAGTGAGCGGCCGC |
| Heavy Chain- | 61. | ATGAAGCACCTGTGGTTCTTTCTGCTGCTGGTGGCTGCTCC |
| 1103 | | AAGATGGGTGCTGTCTGAGATCTCCGAGGTGCAGCTGGTG |
| | | GAGTCCGGAGGAGGACTGGTGCAGCCTGGAGGATCCCTGA |
| | | GACTGAGCTGCGCCGCTTCTGGCTTCAACATCAGCTACTAT |
| | | TCTATCCATTGGGTGCGCCAGGCTCCAGGCAAGGGACTGG |
| | | AGTGGGTGGCCTCCATCTACTCCAGCTCTGGCTACACCTCC |
| | | TATGCCGACAGCGTGAAGGGCAGGTTCACCATCTCTGCCG |
| | | ATACCTCCAAGAACACAGCTTATCTGCAGATGAACTCCCTG |
| | | AGGGCCGAGGACACCGCCGTGTACTATTGTGCTCGGGGCT |
| | | ACGGCCTGGATTATTGGGGCCAGGGCACCCTGGTGACAGT |
| | | GTCCAGCGCTAGCACCAAGGGCCCCTCCGTGTTCCCTCTGG |
| | | CCCCCTGTAGCAGAAGCACAAGCGAGTCCACCGCCGCCCT |
| | | GGGCTGTCTGGTGAAGGACTACTTTCCTGAGCCTGTGACCG |
| | | TGTCCTGGAATAGCGGCGCCCTGACCTCCGGCGTGCACACA |
| | | TTTCCTGCCGTGCTGCAGTCCTCCGGCCTGTACAGCCTGAG |
| | | CAGCGTGGTGACAGTGCCCAGCTCCAACTTCGGCACACAG |
| | | ACATACACCTGCAATGTGGATCACAAGCCTTCCAACACAA |
| | | AGGTGGACAAGACCGTGGAGAGAAAGTGTTGCGTGGAGTG |
| | | CCCTCCTTGCCCCGCCCCTCCAGTGGCTGGACCATCCGTGT |
| | | TCCTGTTTCCCCCTAAGCCCAAGGATACACTGATGATCAGC |
| | | AGGACCCCCGAGGTGACATGCGTGGTGGTGGATGTGAGCC |
| | | ACGAGGACCCCGAGGTGCAGTTTAACTGGTACGTGGACGG |
| | | CGTGGAGGTGCACAACGCCAAGACAAAGCCCAGGGAGGA |
| | | GCAGTTCAATTCCACCTTCAGAGTGGTGAGCGTGCTGACCG |
| | | TGGTGCACCAGGATTGGCTGAATGGCAAGGAGTACAAGTG |
| | | TAAGGTGTCCAACAAGGGCCTGCCCGCCCCCATCGAGAAG |
| | | ACCATCTCCAAGACAAAGGGCCAGCCCAGGGAGCCCCAGG |
| | | TGTACACACTGCCCCCTAGCAGGGAGGAGATGACAAAGAA |
| | | CCAGGTGTCCCTGACCTGCCTGGTGAAGGGCTTCTACCCTA |
| | | GCGACATCAGCGTGGAGTGGGAGTCCAATGGCCAGCCCGA |
| | | GAATAACTACAAGACCACCCCCCCTATGCTGGATAGCGAC |
| | | GGCAGCTTTTTCCTGTACTCCAAGCTGACAGTGGACAAGTC |
| | | CAGGTGGCAGCAGGGCAACGTGTTTAGCTGCAGCGTGATG |
| | | CACGAGGCCCTGCACAACCACTACACCCAGAAGAGCCTGT |
| | | CCCTGTCCCCCGGCAAGTGAGCGGCCGC |
| Heavy Chain- | 62. | ATGAAGCACCTGTGGTTCTTTCTGCTGCTGGTGGCTGCTCC |
| 1104 | | AAGATGGGTGCTGAGCGAGATCTCTGAGGTGCAGCTGGTG |
| | | GAGTCCGGAGGAGGACTGGTGCAGCCTGGAGGATCTCTGA |
| | | GACTGTCCTGCGCTGCCTCCGGCTTCAACATCTCCTACTAT |
| | | AGCATCCATTGGGTGCGCCAGGCTCCAGGCAAGGGACTGG |
| | | AGTGGGTGGCCTCCATCTACTCCAGCTCTTCCAGCACCTAC |
| | | TATGCCGACTCTGTGAAGGGCAGGTTCACCATCTCTGCCGA |
| | | TACCTCCAAGAACACAGCTTATCTGCAGATGAATAGCCTGA |
| | | GGGCCGAGGACACCGCCGTGTACTATTGTGCTCGGGGCTA |
| | | CGGCCTGGATTATTGGGGCCAGGGCACCCTGGTGACAGTG |
| | | TCTTCCGCTAGCACCAAGGGCCCCTCCGTGTTCCCTCTGGC |
| | | CCCCTGTAGCAGAAGCACAAGCGAGTCCACCGCCGCCCTG |
| | | GGCTGTCTGGTGAAGGACTACTTTCCTGAGCCTGTGACCGT |
| | | GTCCTGGAATAGCGGCGCCCTGACCTCCGGCGTGCACACAT |
| | | TTCCTGCCGTGCTGCAGTCCTCCGGCCTGTACAGCCTGAGC |
| | | AGCGTGGTGACAGTGCCCAGCTCCAACTTCGGCACACAGA |
| | | CATACACCTGCAATGTGGATCACAAGCCTTCCAACACAAA |
| | | GGTGGACAAGACCGTGGAGAGAAAGTGTTGCGTGGAGTGC |
| | | CCTCCTTGCCCCGCCCCTCCAGTGGCTGGACCATCCGTGTT |
| | | CCTGTTTCCCCCTAAGCCCAAGGATACACTGATGATCAGCA |
| | | GGACCCCCGAGGTGACATGCGTGGTGGTGGATGTGAGCCA |
| | | CGAGGACCCCGAGGTGCAGTTTAACTGGTACGTGGACGGC |
| | | GTGGAGGTGCACAACGCCAAGACAAAGCCCAGGGAGGAG |
| | | CAGTTCAATTCCACCTTCAGAGTGGTGAGCGTGCTGACCGT |
| | | GGTGCACCAGGATTGGCTGAATGGCAAGGAGTACAAGTGT |
| | | AAGGTGTCCAACAAGGGCCTGCCCGCCCCCATCGAGAAGA |
| | | CCATCTCCAAGACAAAGGGCCAGCCCAGGGAGCCCCAGGT |
| | | GTACACACTGCCCCCTAGCAGGGAGGAGATGACAAAGAAC |
| | | CAGGTGTCCCTGACCTGCCTGGTGAAGGGCTTCTACCCTAG |
| | | CGACATCAGCGTGGAGTGGGAGTCCAATGGCCAGCCCGAG |
| | | AATAACTACAAGACCACCCCCCCTATGCTGGATAGCGACG |
| | | GCAGCTTTTTCCTGTACTCCAAGCTGACAGTGGACAAGTCC |
| | | AGGTGGCAGCAGGGCAACGTGTTTAGCTGCAGCGTGATGC |
| | | ACGAGGCCCTGCACAACCACTACACCCAGAAGAGCCTGTC |
| | | CCTGTCCCCCGGCAAGTGAGCGGCCGC |
| Light Chain- | 63. | ATGGTGCTGCAGACCCAGGTGTTCATCAGCCTGCTGCTGTG |
| 1095 | | GATCTCTGGCGCCTACGGCTCCGACATCCAGATGACACAGT |
| | | CTCCATCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCCGAGGATTTTGCTACATACTATTGT |
| | | CAGCAGCACTACTCCCTGTTCACCTTTGGCCAGGGCACAAA |
| | | GGTGGAGATCAAGcgtacgGTGGCTGCACCTTCTGTGTTCATC |
| | | TTCCCTCCATCTGATGAGCAGCTGAAGTCTGGAACCGCATC |
| | | TGTCGTCTGTCTGCTGAACAACTTTTACCCCAGGGAGGCTA |
| | | AGGTCCAATGGAAGGTGGACAACGCCCTGCAGTCTGGTAA |
| | | TAGCCAGGAAAGCGTGACCGAACAGGATTCCAAGGACTCC |
| | | ACCTACTCCCTGTCCTCCACACTGACACTGAGCAAAGCCGA |
| | | CTATGAAAAGCACAAAGTGTATGCCTGCGAGGTCACTCAT |
| | | CAGGGCCTGTCCAGCCCCGTGACTAAAAGCTTTAATAGGG |
| | | GGGAGTGCTGAGCGGCCGC |
| Light Chain- | 64. | ATGGTGCTGCAGACCCAGGTGTTCATCAGCCTGCTGCTGTG |
| 1096 | | GATCTCTGGCGCCTACGGCTCCGACATCCAGATGACACAGT |
| | | CTCCTTCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCAGAGGATTTCGCCACATACTATTGT |
| | | CAGCAGGGCTTTGCTCCCTTCACCTTTGGCCAGGGCACAAA |
| | | GGTGGAGATCAAGcgtacgGTGGCTGCACCTTCTGTGTTCATC |
| | | TTCCCTCCATCTGATGAGCAGCTGAAGTCTGGAACCGCATC |
| | | TGTCGTCTGTCTGCTGAACAACTTTTACCCCAGGGAGGCTA |
| | | AGGTCCAATGGAAGGTGGACAACGCCCTGCAGTCTGGTAA |
| | | TAGCCAGGAAAGCGTGACCGAACAGGATTCCAAGGACTCC |
| | | ACCTACTCCCTGTCCTCCACACTGACACTGAGCAAAGCCGA |
| | | CTATGAAAAGCACAAAGTGTATGCCTGCGAGGTCACTCAT |
| | | CAGGGCCTGTCCAGCCCCGTGACTAAAAGCTTTAATAGGG |
| | | GGGAGTGCTGAGCGGCCGC |
| Light Chain- | 65. | ATGGTGCTGCAGACCCAGGTGTTCATCAGCCTGCTGCTGTG |
| 1097 | | GATCTCTGGCGCCTACGGCTCCGACATCCAGATGACACAGT |
| | | CTCCTTCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCAGAGGATTTTGCTACATACTATTGT |
| | | CAGCAGTCCTACGGCCCCTTCACCTTTGGCCAGGGCACAAA |
| | | GGTGGAGATCAAGcgtacgGTGGCTGCACCTTCTGTGTTCATC |
| | | TTCCCTCCATCTGATGAGCAGCTGAAGTCTGGAACCGCATC |
| | | TGTCGTCTGTCTGCTGAACAACTTTTACCCCAGGGAGGCTA |
| | | AGGTCCAATGGAAGGTGGACAACGCCCTGCAGTCTGGTAA |
| | | TAGCCAGGAAAGCGTGACCGAACAGGATTCCAAGGACTCC |
| | | ACCTACTCCCTGTCCTCCACACTGACACTGAGCAAAGCCGA |
| | | CTATGAAAAGCACAAAGTGTATGCCTGCGAGGTCACTCAT |
| | | CAGGGCCTGTCCAGCCCCGTGACTAAAAGCTTTAATAGGG |
| | | GGGAGTGCTGAGCGGCCGC |
| Light Chain- | 66. | ATGGTGCTGCAGACCCAGGTGTTCATCTCCCTGCTGCTGTG |
| 1098 | | GATCAGCGGCGCCTACGGCTCTGACATCCAGATGACACAG |
| | | AGCCCTTCCAGCCTGTCTGCCTCCGTGGGCGATAGAGTGAC |
| | | CATCACATGCAGGGCTAGCCAGTCCGTGTCCTCCGCCGTGG |
| | | CTTGGTATCAGCAGAAGCCCGGCAAGGCCCCTAAGCTGCT |
| | | GATCTACTCTGCTAGCTCTCTGTATTCTGGAGTGCCATCCA |
| | | GGTTCTCCGGAAGCCGGTCTGGAACCGACTTTACCCTGACA |
| | | ATCTCCAGCCTGCAGCCAGAGGATTTTGCTACATACTATTG |
| | | TCAGCAGTCCTACAGCCCCTTCACCTTTGGCCAGGGCACAA |
| | | AGGTGGAGATCAAGcgtacgGTGGCTGCACCTTCTGTGTTCAT |
| | | CTTCCCTCCATCTGATGAGCAGCTGAAGTCTGGAACCGCAT |
| | | CTGTCGTCTGTCTGCTGAACAACTTTTACCCCAGGGAGGCT |
| | | AAGGTCCAATGGAAGGTGGACAACGCCCTGCAGTCTGGTA |
| | | ATAGCCAGGAAAGCGTGACCGAACAGGATTCCAAGGACTC |
| | | CACCTACTCCCTGTCCTCCACACTGACACTGAGCAAAGCCG |
| | | ACTATGAAAAGCACAAAGTGTATGCCTGCGAGGTCACTCA |
| | | TCAGGGCCTGTCCAGCCCCGTGACTAAAAGCTTTAATAGGG |
| | | GGGAGTGCTGAGCGGCCGC |
| Light Chain- | 67. | ATGGTGCTGCAGACCCAGGTGTTCATCAGCCTGCTGCTGTG |
| 1099 | | GATCTCTGGCGCCTACGGCTCCGACATCCAGATGACACAGT |
| | | CTCCATCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCCGAGGATTTTGCCACATACTATTGT |
| | | CAGCAGCACTACGCTCTGTTCACCTTTGGCCAGGGCACAAA |
| | | GGTGGAGATCAAGcgtacgGTGGCTGCACCTTCTGTGTTCATC |
| | | TTCCCTCCATCTGATGAGCAGCTGAAGTCTGGAACCGCATC |
| | | TGTCGTCTGTCTGCTGAACAACTTTTACCCCAGGGAGGCTA |
| | | AGGTCCAATGGAAGGTGGACAACGCCCTGCAGTCTGGTAA |
| | | TAGCCAGGAAAGCGTGACCGAACAGGATTCCAAGGACTCC |
| | | ACCTACTCCCTGTCCTCCACACTGACACTGAGCAAAGCCGA |
| | | CTATGAAAAGCACAAAGTGTATGCCTGCGAGGTCACTCAT |
| | | CAGGGCCTGTCCAGCCCCGTGACTAAAAGCTTTAATAGGG |
| | | GGGAGTGCTGAGCGGCCGC |
| Light Chain- | 68. | ATGGTGCTGCAGACCCAGGTGTTCATCAGCCTGCTGCTGTG |
| 1100 | | GATCTCTGGCGCCTACGGCTCCGACATCCAGATGACACAGT |
| | | CTCCATCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCCGAGGATTTCGCTACATACTATTGT |
| | | CAGCAGCACTACGGCCTGATCACCTTTGGCCAGGGCACAA |
| | | AGGTGGAGATCAAGcgtacgGTGGCTGCACCTTCTGTGTTCAT |
| | | CTTCCCTCCATCTGATGAGCAGCTGAAGTCTGGAACCGCAT |
| | | CTGTCGTCTGTCTGCTGAACAACTTTTACCCCAGGGAGGCT |
| | | AAGGTCCAATGGAAGGTGGACAACGCCCTGCAGTCTGGTA |
| | | ATAGCCAGGAAAGCGTGACCGAACAGGATTCCAAGGACTC |
| | | CACCTACTCCCTGTCCTCCACACTGACACTGAGCAAAGCCG |
| | | ACTATGAAAAGCACAAAGTGTATGCCTGCGAGGTCACTCA |
| | | TCAGGGCCTGTCCAGCCCCGTGACTAAAAGCTTTAATAGGG |
| | | GGGAGTGCTGAGCGGCCGC |
| Light Chain- | 69. | ATGGTGCTGCAGACCCAGGTGTTCATCAGCCTGCTGCTGTG |
| 1101 | | GATCTCTGGCGCCTACGGCTCCGACATCCAGATGACACAGT |
| | | CTCCTTCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCAGAGGATTTCGCCACATACTATTGT |
| | | CAGCAGGCTGGCTGGCCCATCACCTTTGGCCAGGGCACAA |
| | | AGGTGGAGATCAAGcgtacgGTGGCTGCACCTTCTGTGTTCAT |
| | | CTTCCCTCCATCTGATGAGCAGCTGAAGTCTGGAACCGCAT |
| | | CTGTCGTCTGTCTGCTGAACAACTTTTACCCCAGGGAGGCT |
| | | AAGGTCCAATGGAAGGTGGACAACGCCCTGCAGTCTGGTA |
| | | ATAGCCAGGAAAGCGTGACCGAACAGGATTCCAAGGACTC |
| | | CACCTACTCCCTGTCCTCCACACTGACACTGAGCAAAGCCG |
| | | ACTATGAAAAGCACAAAGTGTATGCCTGCGAGGTCACTCA |
| | | TCAGGGCCTGTCCAGCCCCGTGACTAAAAGCTTTAATAGGG |
| | | GGGAGTGCTGAGCGGCCGC |
| Light Chain- | 70. | ATGGTGCTGCAGACCCAGGTGTTCATCAGCCTGCTGCTGTG |
| 1102 | | GATCTCTGGCGCCTACGGCTCCGACATCCAGATGACACAGT |
| | | CTCCTTCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCAGAGGATTTTGCCACATACTATTGT |
| | | CAGCAGGCTTACTCCCCCTTCACCTTTGGCCAGGGCACAAA |
| | | GGTGGAGATCAAGcgtacgGTGGCTGCACCTTCTGTGTTCATC |
| | | TTCCCTCCATCTGATGAGCAGCTGAAGTCTGGAACCGCATC |
| | | TGTCGTCTGTCTGCTGAACAACTTTTACCCCAGGGAGGCTA |
| | | AGGTCCAATGGAAGGTGGACAACGCCCTGCAGTCTGGTAA |
| | | TAGCCAGGAAAGCGTGACCGAACAGGATTCCAAGGACTCC |
| | | ACCTACTCCCTGTCCTCCACACTGACACTGAGCAAAGCCGA |
| | | CTATGAAAAGCACAAAGTGTATGCCTGCGAGGTCACTCAT |
| | | CAGGGCCTGTCCAGCCCCGTGACTAAAAGCTTTAATAGGG |
| | | GGGAGTGCTGAGCGGCCGC |
| Light Chain- | 71. | ATGGTGCTGCAGACCCAGGTGTTCATCAGCCTGCTGCTGTG |
| 1103 | | GATCTCTGGCGCCTACGGCTCCGACATCCAGATGACACAGT |
| | | CTCCTTCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCAGAGGATTTCGCTACATACTATTGT |
| | | CAGCAGCACTACTCCTATCCCATCACCTTTGGCCAGGGCAC |
| | | AAAGGTGGAGATCAAGcgtacgGTGGCTGCACCTTCTGTGTTC |
| | | ATCTTCCCTCCATCTGATGAGCAGCTGAAGTCTGGAACCGC |
| | | ATCTGTCGTCTGTCTGCTGAACAACTTTTACCCCAGGGAGG |
| | | CTAAGGTCCAATGGAAGGTGGACAACGCCCTGCAGTCTGG |
| | | TAATAGCCAGGAAAGCGTGACCGAACAGGATTCCAAGGAC |
| | | TCCACCTACTCCCTGTCCTCCACACTGACACTGAGCAAAGC |
| | | CGACTATGAAAAGCACAAAGTGTATGCCTGCGAGGTCACT |
| | | CATCAGGGCCTGTCCAGCCCCGTGACTAAAAGCTTTAATAG |
| | | GGGGGAGTGCTGAGCGGCCGC |
| Light Chain- | 72. | ATGGTGCTGCAGACCCAGGTGTTCATCAGCCTGCTGCTGTG |
| 1104 | | GATCTCTGGCGCCTACGGCTCCGACATCCAGATGACACAGT |
| | | CTCCATCCAGCCTGTCTGCCTCCGTGGGCGATAGAGTGACC |
| | | ATCACATGCAGGGCTAGCCAGTCCGTGTCCTCCGCCGTGGC |
| | | TTGGTATCAGCAGAAGCCCGGCAAGGCCCCTAAGCTGCTG |
| | | ATCTACTCCGCTAGCTCTCTGTATTCCGGAGTGCCAAGCAG |
| | | GTTCTCCGGAAGCCGGTCTGGAACCGACTTTACCCTGACAA |
| | | TCTCCAGCCTGCAGCCCGAGGATTTCGCTACATACTATTGT |
| | | CAGCAGGCCTACGCTCTGATCACCTTTGGCCAGGGCACAA |
| | | AGGTGGAGATCAAGcgtacgGTGGCTGCACCTTCTGTGTTCAT |
| | | CTTCCCTCCATCTGATGAGCAGCTGAAGTCTGGAACCGCAT |
| | | CTGTCGTCTGTCTGCTGAACAACTTTTACCCCAGGGAGGCT |
| | | AAGGTCCAATGGAAGGTGGACAACGCCCTGCAGTCTGGTA |
| | | ATAGCCAGGAAAGCGTGACCGAACAGGATTCCAAGGACTC |
| | | CACCTACTCCCTGTCCTCCACACTGACACTGAGCAAAGCCG |
| | | ACTATGAAAAGCACAAAGTGTATGCCTGCGAGGTCACTCA |
| | | TCAGGGCCTGTCCAGCCCCGTGACTAAAAGCTTTAATAGGG |
| | | GGGAGTGCTGAGCGGCCGC |

Selected results of the experiments are now described.

### Example 1: Identification of Unc5B as a Regulator of the Blood Brain Barrier

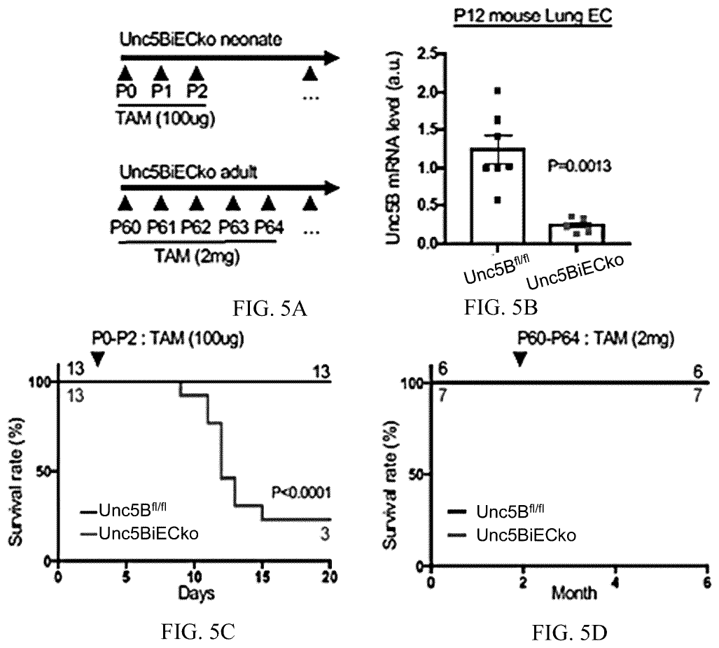

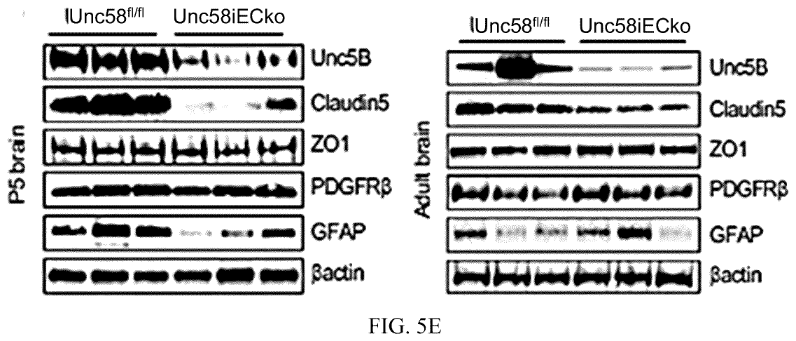

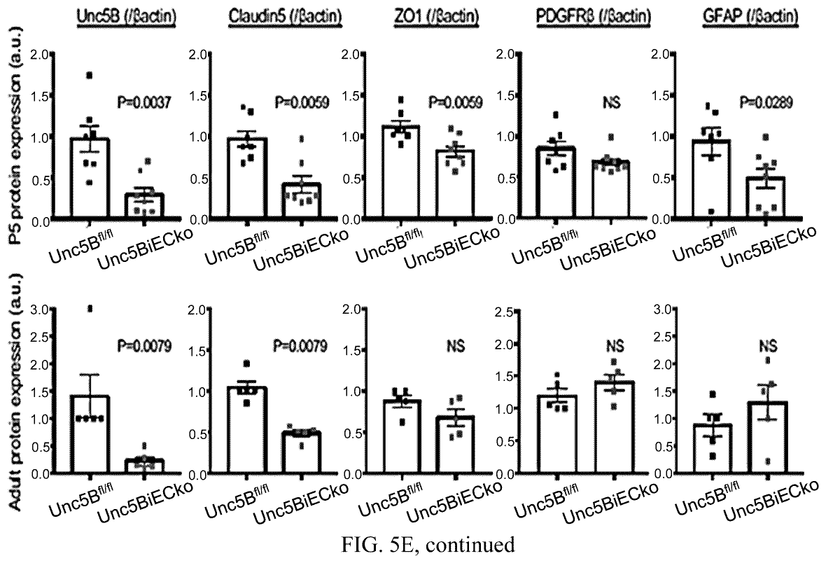

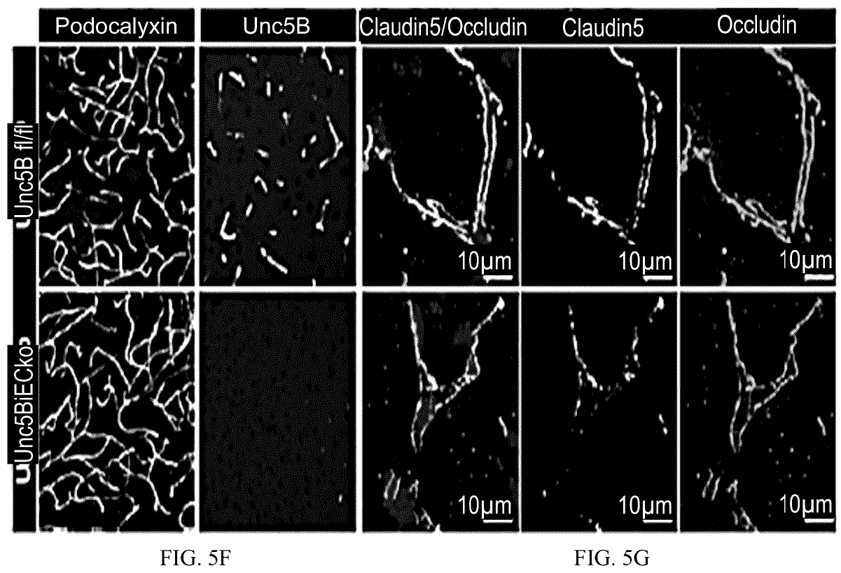

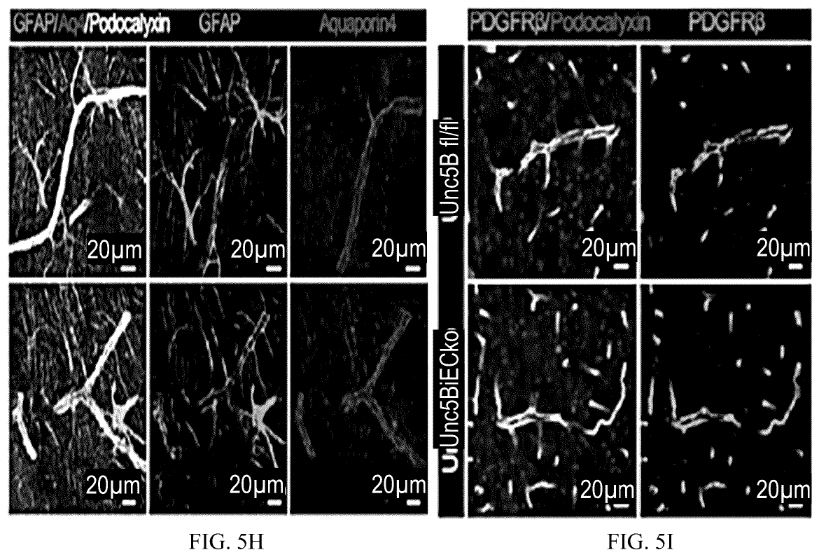

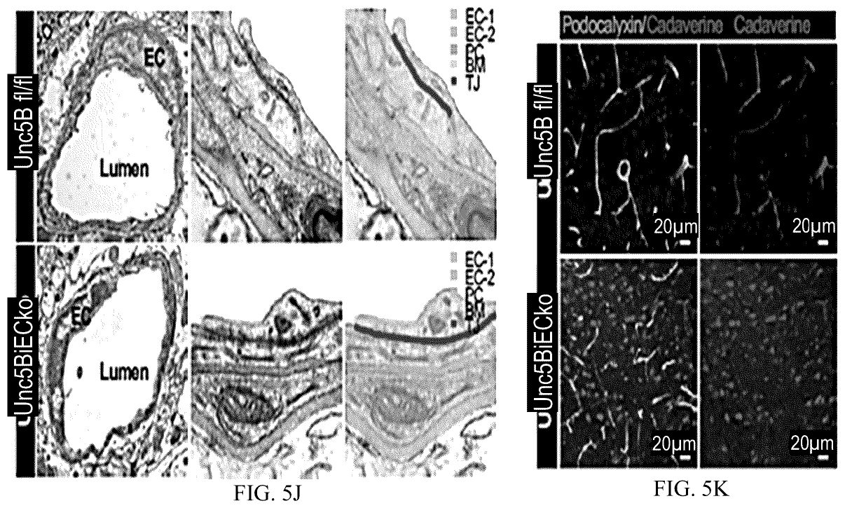

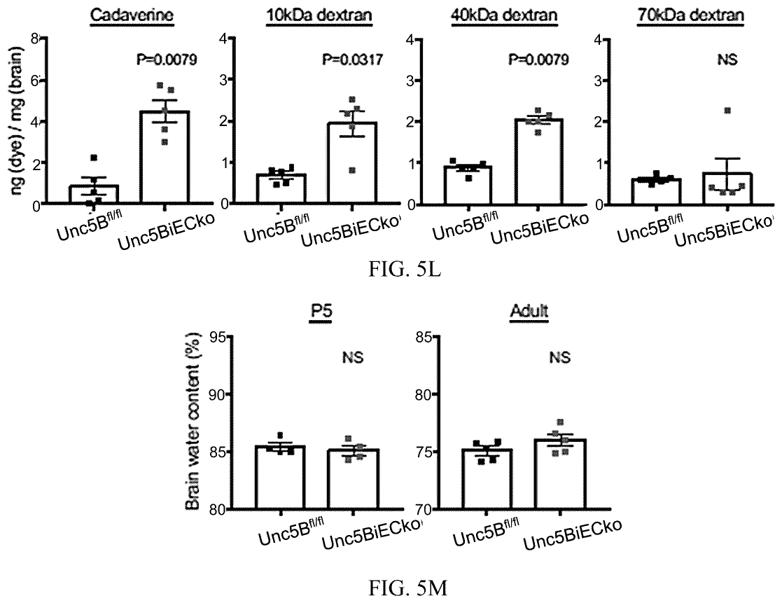

To investigate postnatal and adult Unc5B function, temporally inducible, endothelial specific Unc5B knockout mice were generated by crossing Unc5Bfl/fl mice with Cdh5CreERT2 mice (hereafter Unc5BiECko). Gene deletion was induced by Tamoxifen (TAM) injection in neonates starting at postnatal day (P) 0-2, or in adult mice (FIG. 5A), and was efficient as revealed by qPCR, Western blot and antibody staining (FIGS. 5B, 5E, 5F). Neonatal Unc5B deletion induced severe neurological defects and lethality around p15 (FIG. 5C), whereas adult Unc5B deletion had no effect on mouse weight or survival (FIG. 5D).

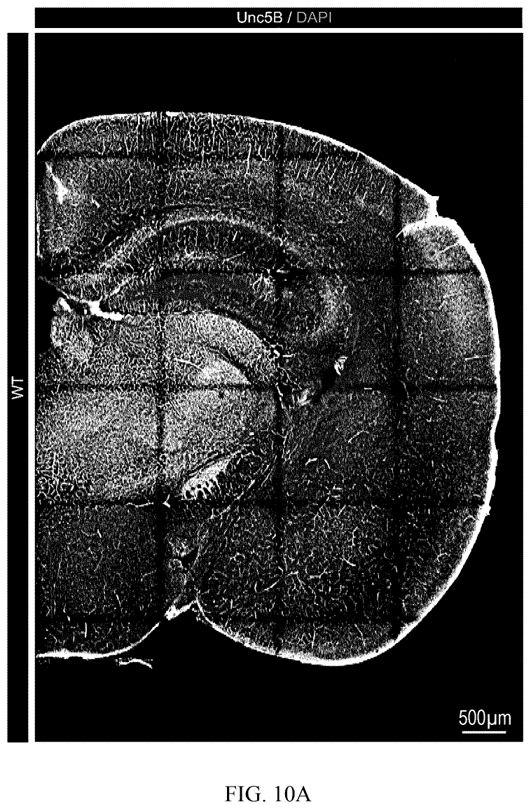

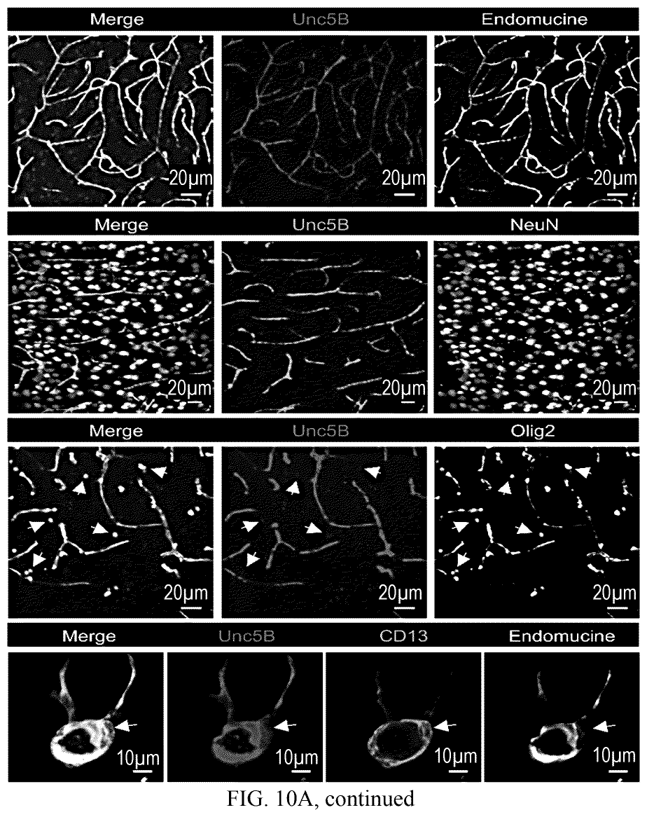

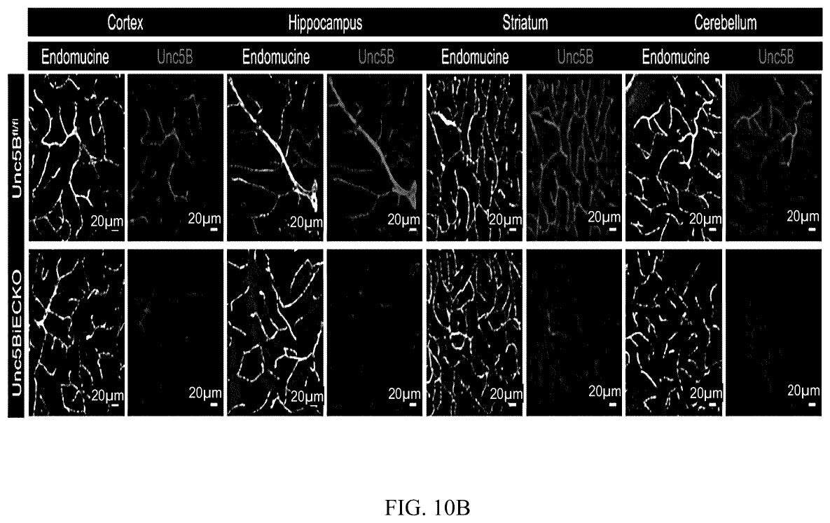

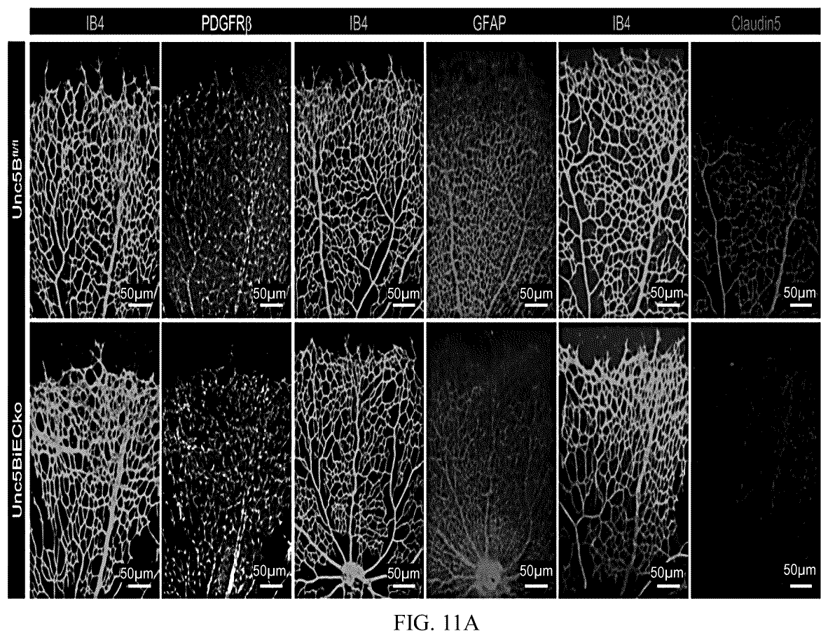

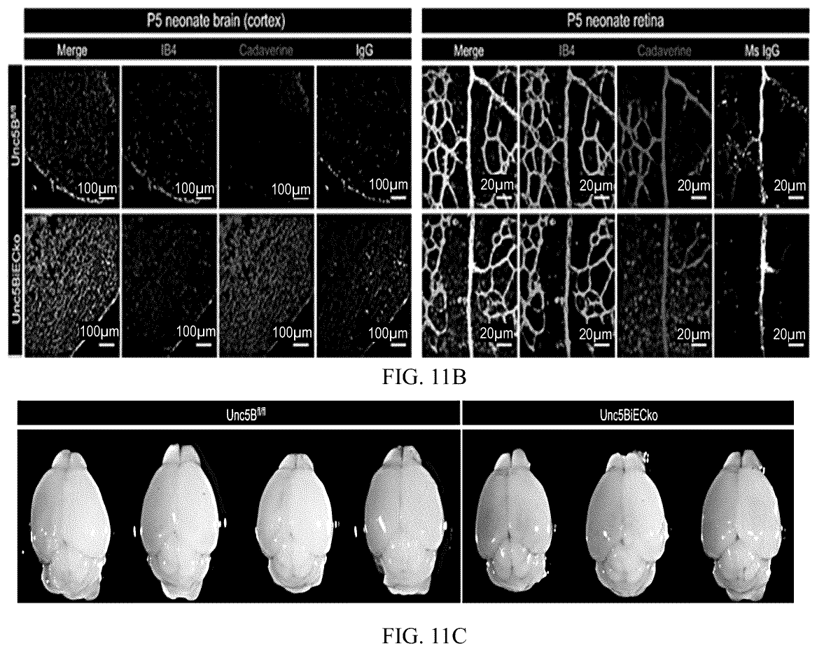

To understand the effects of Unc5B in postnatal and adult brain, its expression in the CNS vasculature was examined. In adult control brains, an anti-Unc5B antibody preferentially labeled arterial endothelial cells but was also found at lower levels in veins and capillaries in various regions including the cortex, hippocampus, striatum and cerebellum (FIG. 10A). The anti-Unc5B staining was strongly reduced in Unc5BiECko mice (FIG. 5F and FIG. 10B). Neonatal Unc5B deletion slightly increased retinal vessel sprouting (FIG. 11A). Adult Unc5B deletion did not affect the brain vascular architecture (FIG. 5F). However, the levels of Claudin5 were strongly reduced at all analyzed stages, whereas Occludin staining was similar between groups (FIG. 5G). TJs were examined by transmission electron microscopy (TEM) and found that TJs were present in their normal location in endothelial cells. However, they exhibited a more diffuse and electron-dense appearance, similar to the morphology reported in Claudin-5 knockout mice (FIG. 5J).

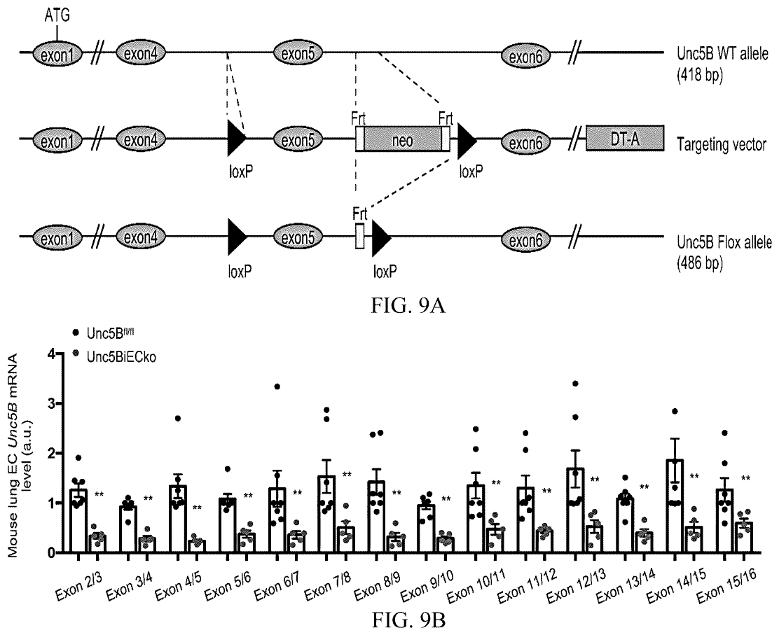

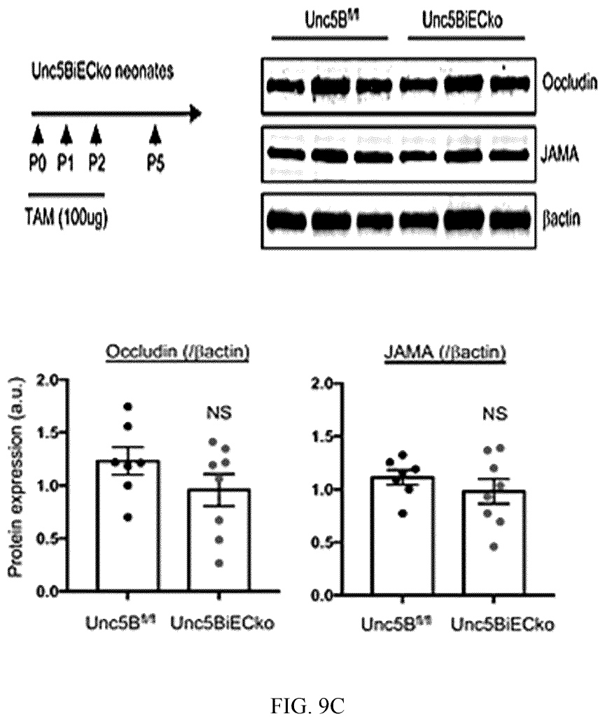

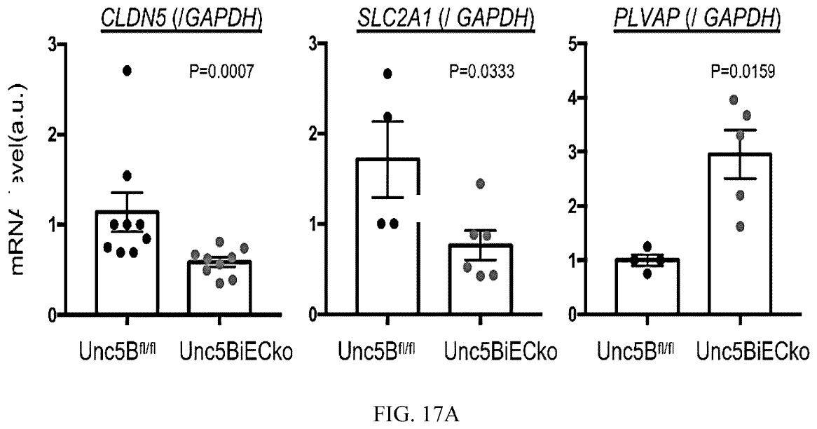

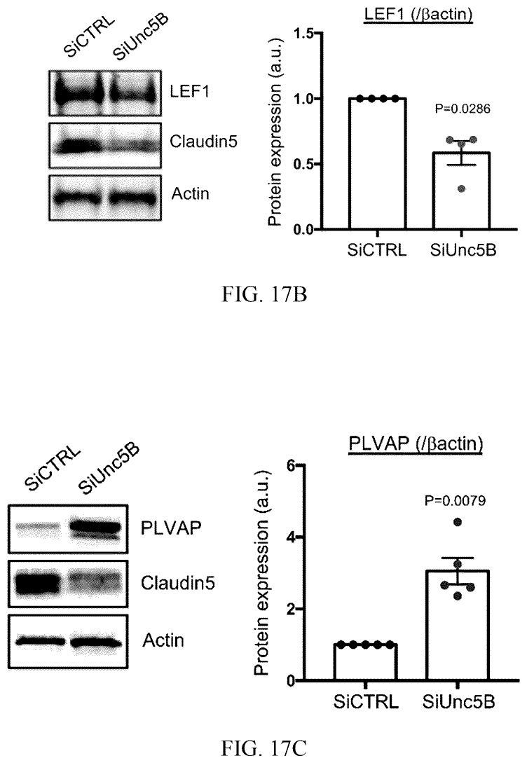

Next, studies examined the consequences of endothelial Unc5B deletion on the assembly of the neurovascular unit. GFAP and Aquaporin-4 expressed by astrocytes and astrocyte end feet, respectively, and PDGFRb expressed by pericytes, were observed, and they were all similar between the two genotypes in adult mice (FIGS. 5H-51). TEM also showed that astrocytes, pericytes and basal lamina were present in adult Unc5BiECko mice and their ultrastructure appeared normal (FIG. 5J). Overall, this analysis revealed that endothelial Unc5B deletion mainly reduced expression of Claudin-5 at the BBB TJs.

To determine if the observed molecular changes translated into a BBB defect, BBB integrity was probed by i.v. injection of tracers with different molecular weights. Leakage of cadaverine (MW 950 Da) into the postnatal and adult Unc5BiECko brains and retinas was significantly increased when compared to controls (FIG. 5K and FIGS. 11B-11C), demonstrating that loss of Unc5B compromised both postnatal and adult BBB integrity. To test if the BBB defect was size selective, mice were injected with fluorescent Dextrans of increasing molecular weights. It was found that 10 kDa and 40 kDa Dextrans also leaked more into the brain of Unc5BiECko mice compared to controls, whereas 70 kDa Dextran did not leak into the mutant mice, suggesting a size exclusion for proteins greater than 40 kDa (FIG. 5L). Endogenous IgG (150 kDa) also remained confined to vessels in both controls and mutants (FIG. 11B). Moreover, no intracranial bleeding was observed upon Unc5B deletion, and brain water content was similar between Cre negative and Cre positive mice at 1 week after induction of gene deletion (FIG. 5M). Hence, genetic loss of Unc5B function induced a size-selective BBB defect in postnatal and adult mice.

### Example 2: Development of Antibody Blockade of Unc5B

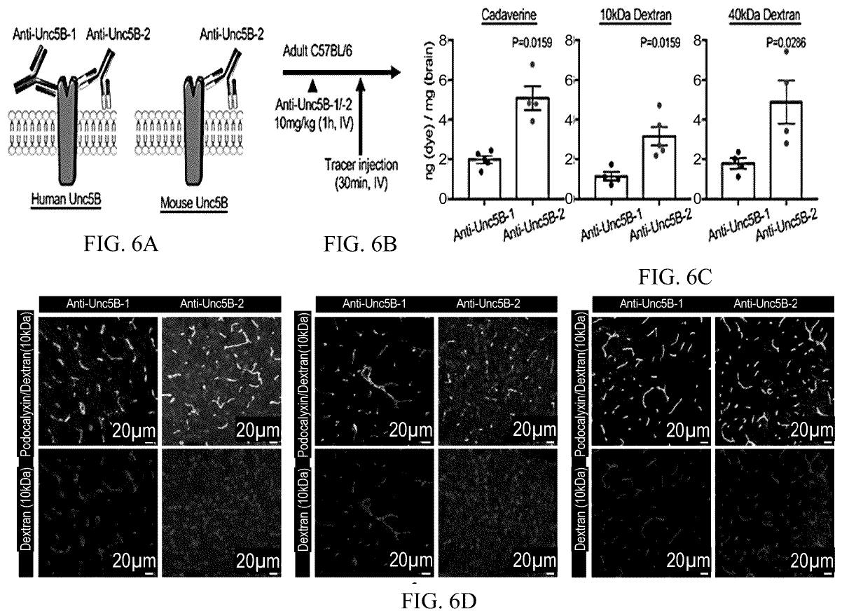

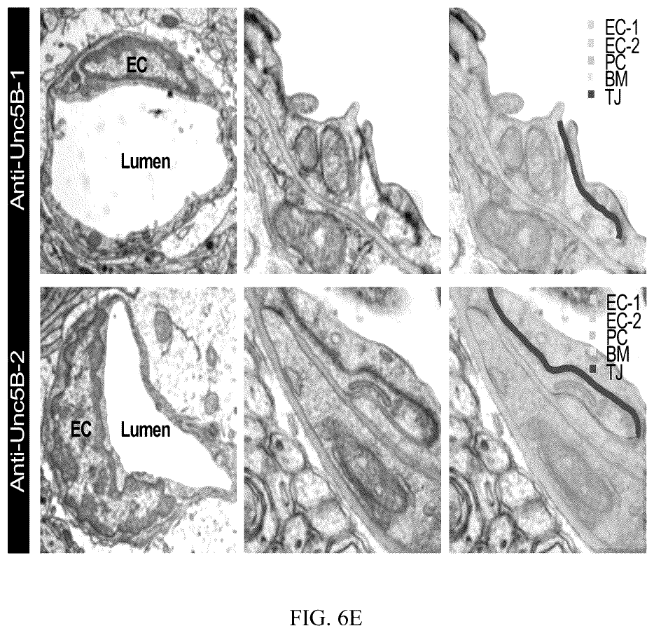

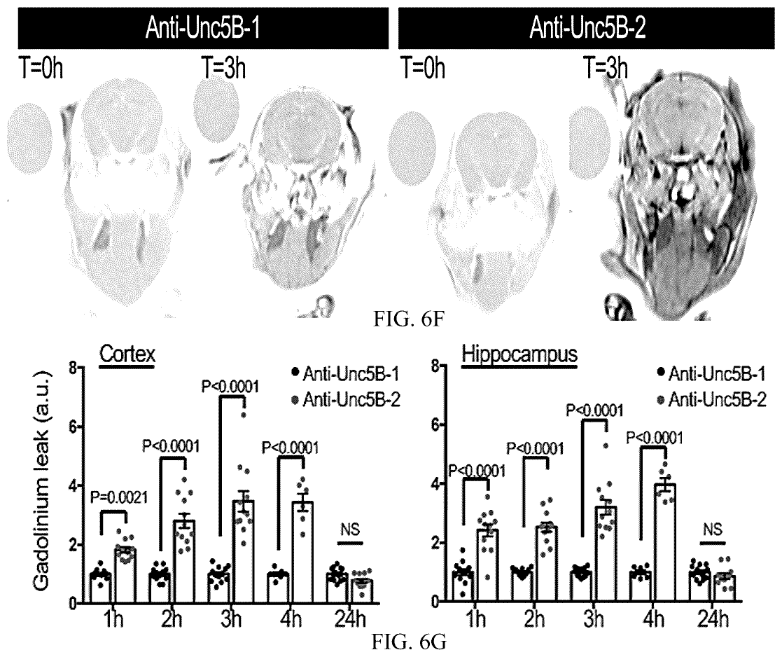

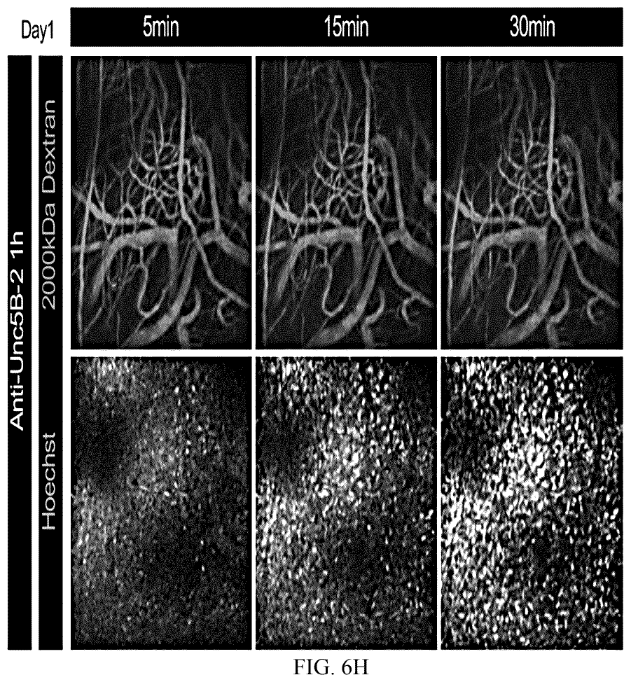

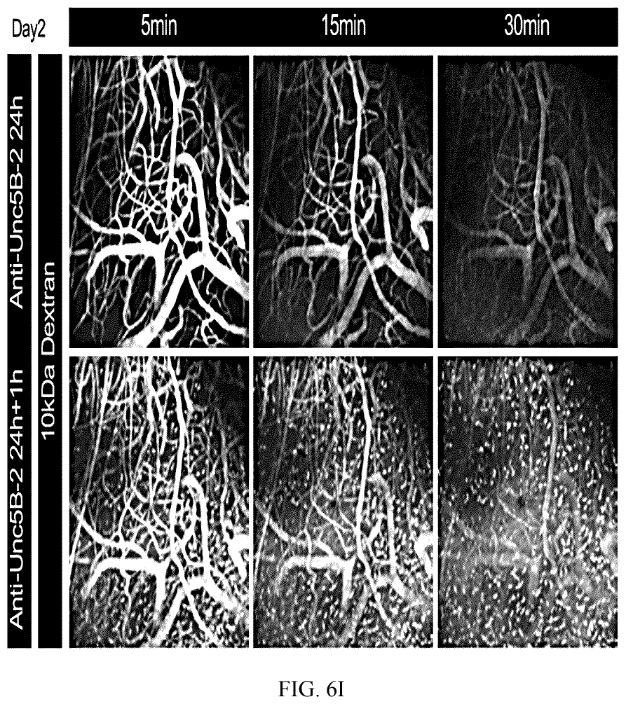

Without wishing to be bound by theory, it was hypothesized that antibody mediated Unc5B blockade could be an approach to transiently block receptor function for on-demand BBB opening. To test this idea, two monoclonal antibodies were used, one control antibody recognizing human but not mouse Unc5B (anti-Unc5B-1), and one blocking mouse and human Unc5B function (anti-Unc5B-2) (FIG. 6A). These antibodies bind with high affinity to the Unc5B extracellular domain and induce receptor internalization, thereby blocking Unc5B function.

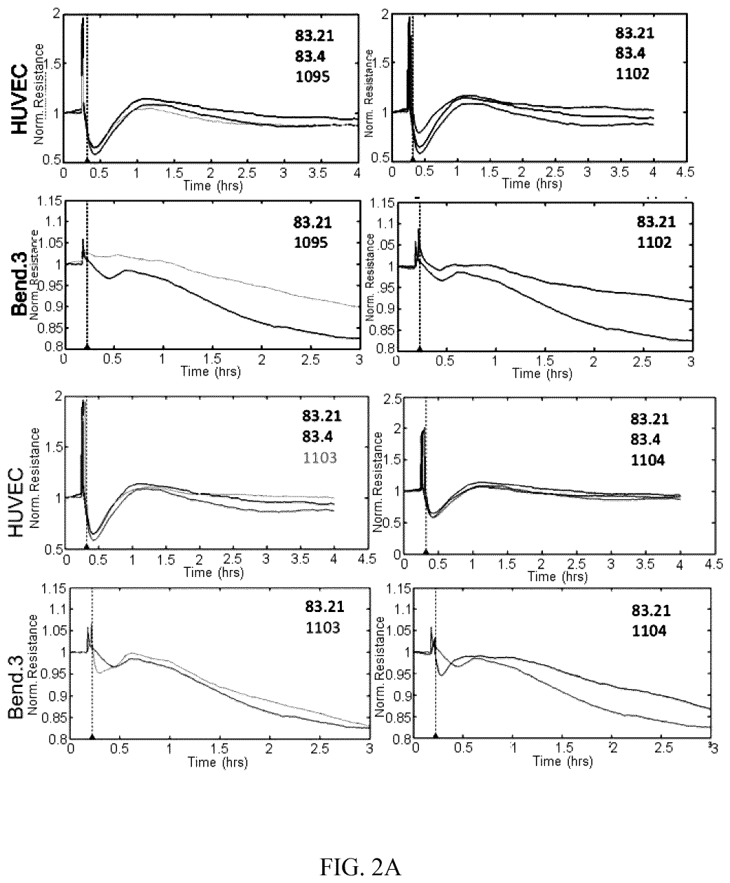

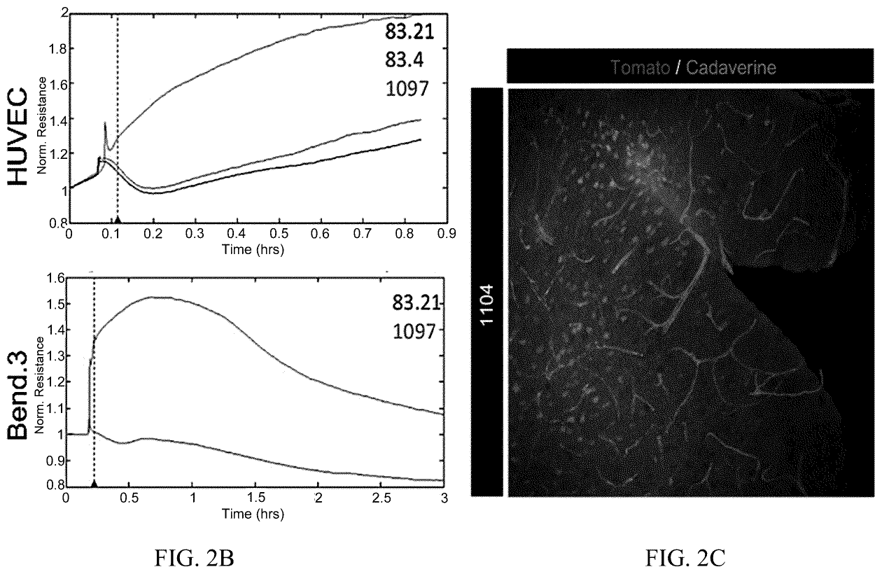

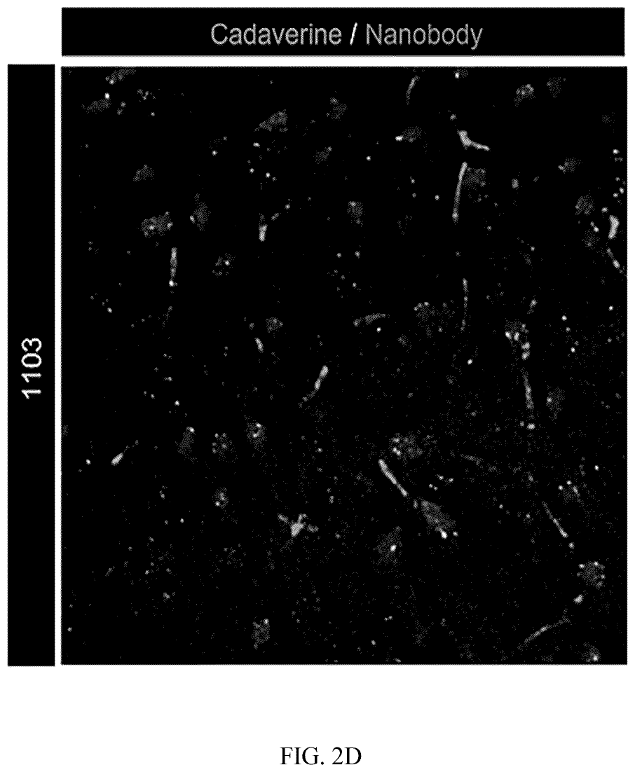

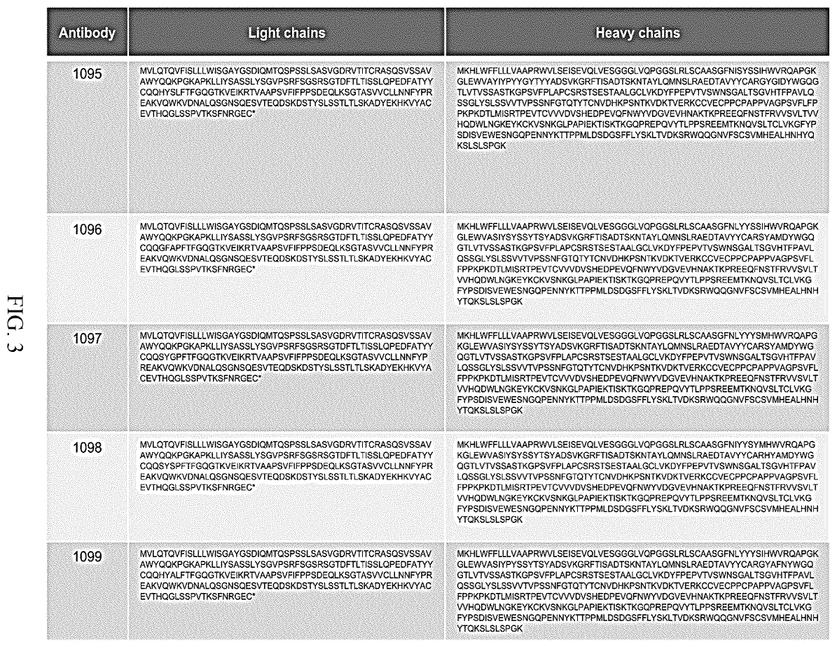

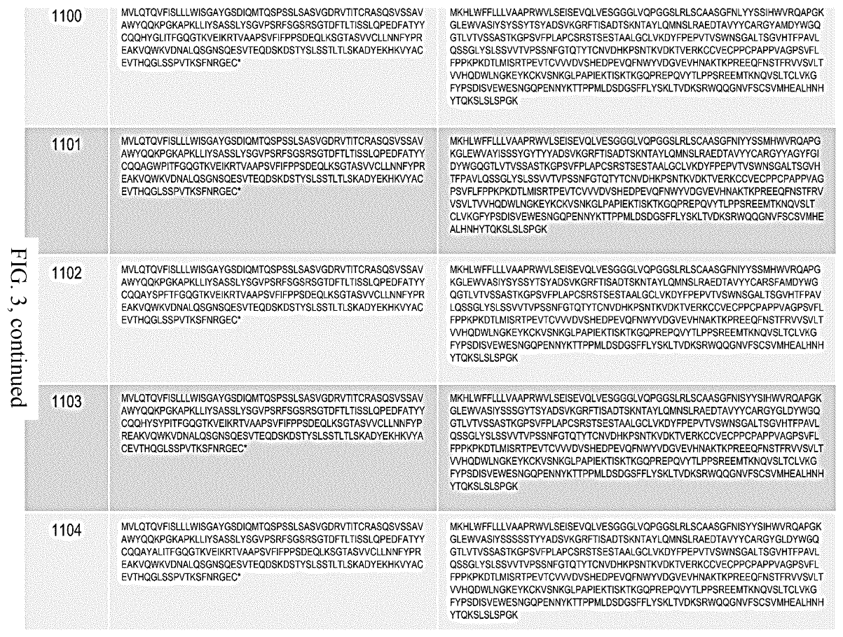

Adult WT mice were injected intravenously (iv) with anti-Unc5B-1 and -2 (10 mg/kg) for 1 hr, followed by iv injection of tracers of various molecular weights 30 min before sacrifice and analysis (FIG. 2B). In mice treated with control anti-Unc5B-1, there were no signs of BBB disruption and all injected tracers remained confined within the neurovasculature (FIGS. 6C-6D).

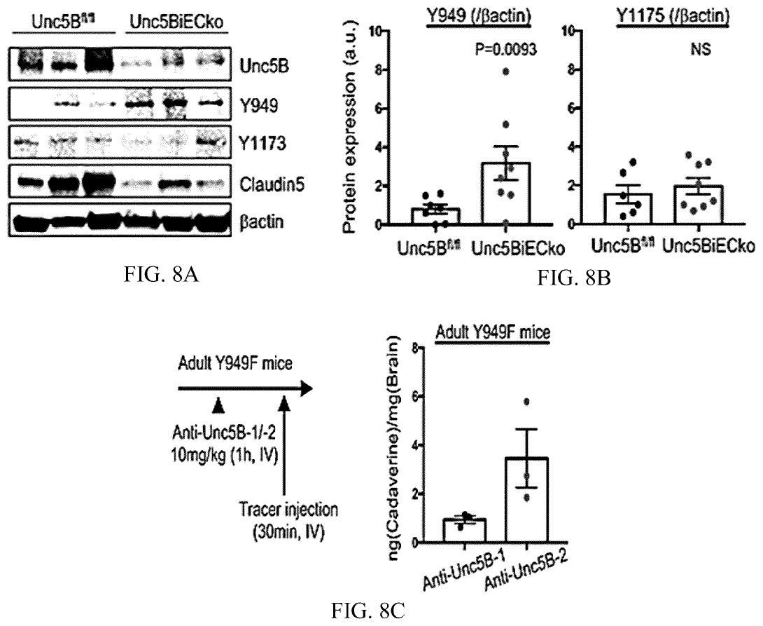

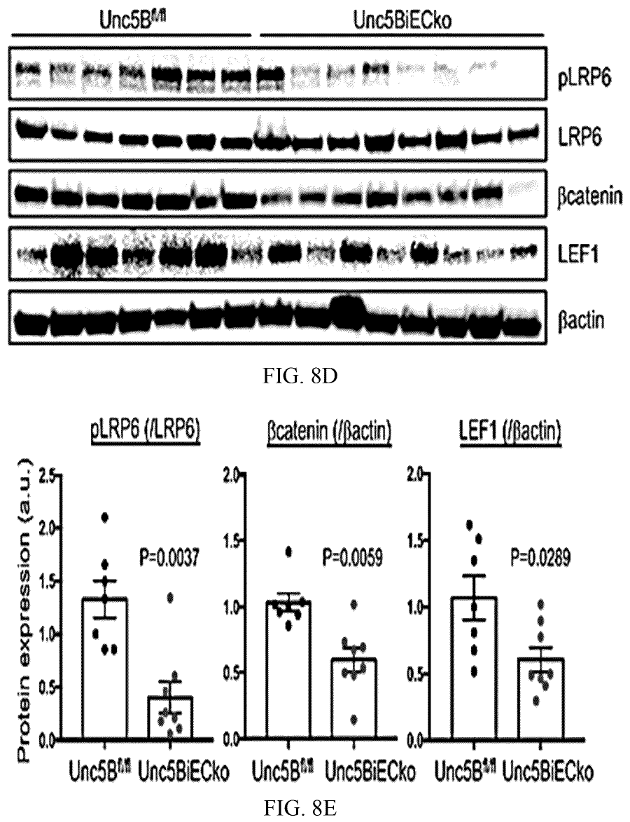

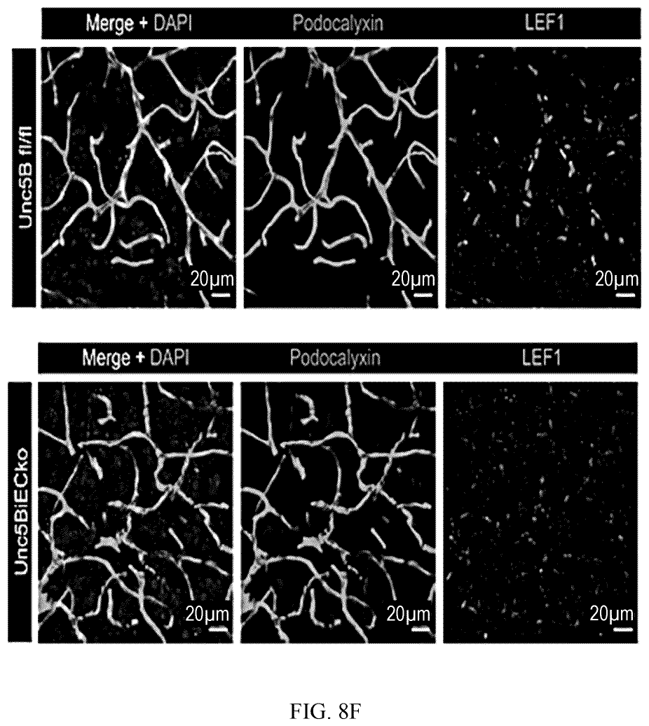

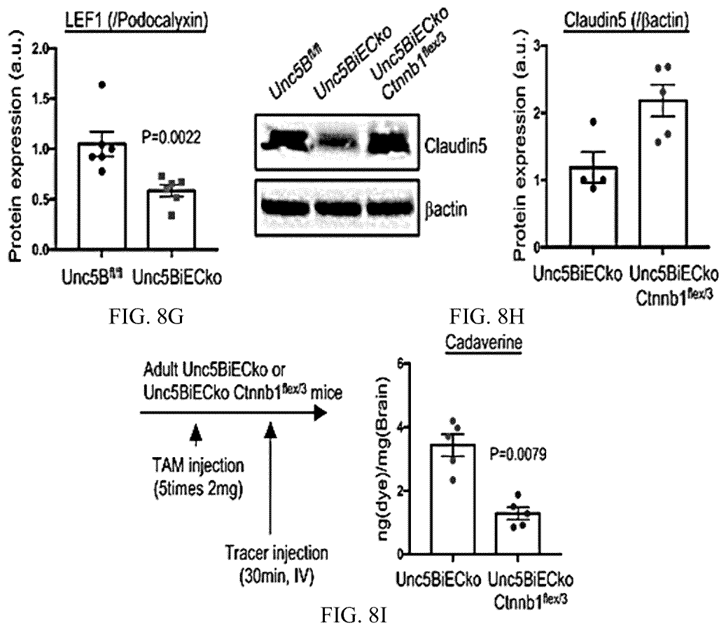

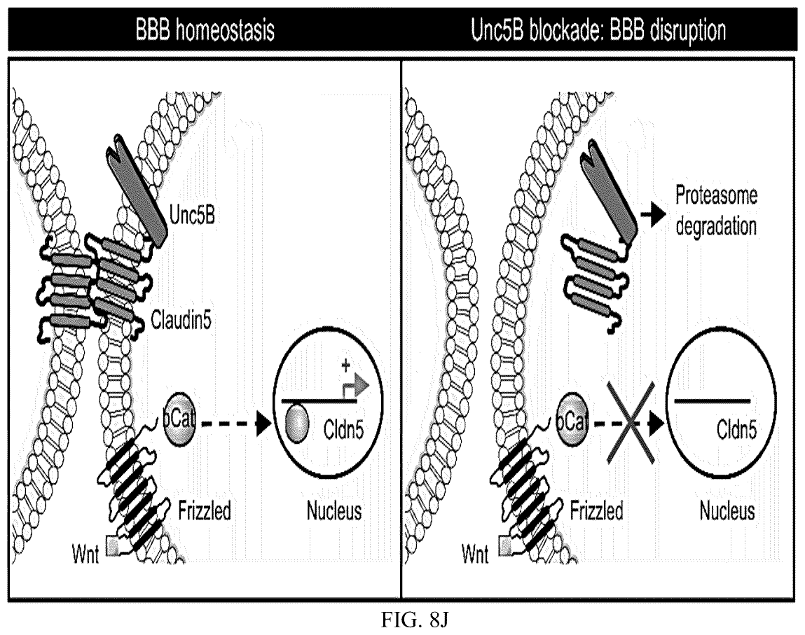

In contrast, mice treated with anti-Unc5B-2 showed a significant leakage of injected Cadaverine into the brain parenchyma (FIGS. 6C-6D). Anti-Unc5B-2 also induced leakage of 10- and 40, but not 70 kDa Dextran or endogenous IgG and fibrinogen (FIG. 8A). Thus, the 30) size-selectivity of the BBB leakage was similar between genetic and antibody-mediated Unc5B inhibition. The vascular barrier-disrupting effect of Unc5B was specific to the brain, as 10 kDa Dextran leak in other organs such as the lung, kidney, thymus, heart, liver and stomach was similar in anti-Unc5B-1 and -2 injected mice (FIG. 8B). Moreover, tracer leakage was found in all CNS areas examined including cortex, striatum and cerebellum (FIG. 8C). TEM was performed to examine ultrastructure of the blood vessels in anti-Unc5B-1 and -2 treated brains from multiple animals. The result show that morphology of pericytes, astrocytes and basement membrane are very similar between groups (FIG. 8E). TJs in anti-Unc5B treated mice resembled those of Unc5BiECko mice (FIGS. 5J and 6E).

It was next determined if and how fast BBB resealing occurred after an anti-Unc5B antibody injection, using both MRI and two-photon live imaging through cranial windows. Mice received one iv injection of anti-Unc5B-1 or -2 (10 mg/kg) and were imaged by contrast MRI after iv injection of gadoteric acid (MW 558 Da) at 1, 2, 3, 4 and 24 hours following antibody treatment. Quantification showed a significant tracer leakage in the cortex and hippocampus between 1-46 hours after anti-Unc5B-2 delivery, which returned to baseline levels after 24 hours (FIGS. 6F-6G), indicating that the neurovascular barrier had resealed.

Two photon live imaging showed that a 2000 kDa FITC-dextran did not leak at any time point after anti-Unc5B-2 treatment, but outlined the brain vasculature (FIG. 6H). By contrast, Hoechst (MW 560 Da) started to extravasate from superficial cortical vessels within 5 min after injection and continued to leak over 30 minutes in mice that were treated with the anti-Unc5B-2 antibody one hour prior to tracer injection (FIG. 6H). When 10 kDa dextran was injected intravenously in mice that were treated with the anti-Unc5B-2 antibody 24 hours earlier, tracer leakage was not observed (FIG. 6I), indicating that the BBB had resealed. However, re-administration of a second dose of anti-Unc5B-2 re-opened the barrier within an hour leading to 10 kDa dextran extravasation over the next 30 min (FIG. 6I). Altogether, these data showed that anti-Unc5B-2 could be used to reversibly open the BBB to tracers up to 40 kDa, which is compatible with delivery of small molecules and antibody therapeutics such as nanobodies.

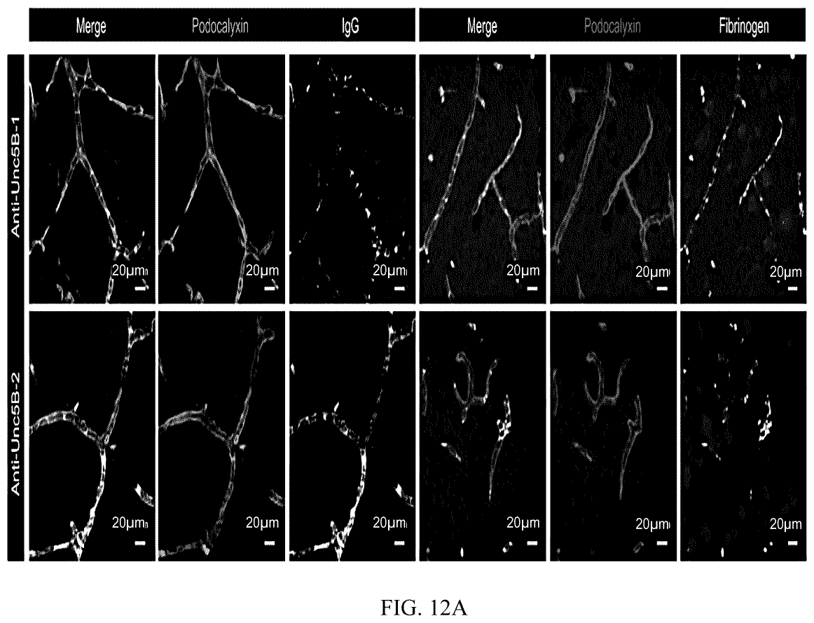

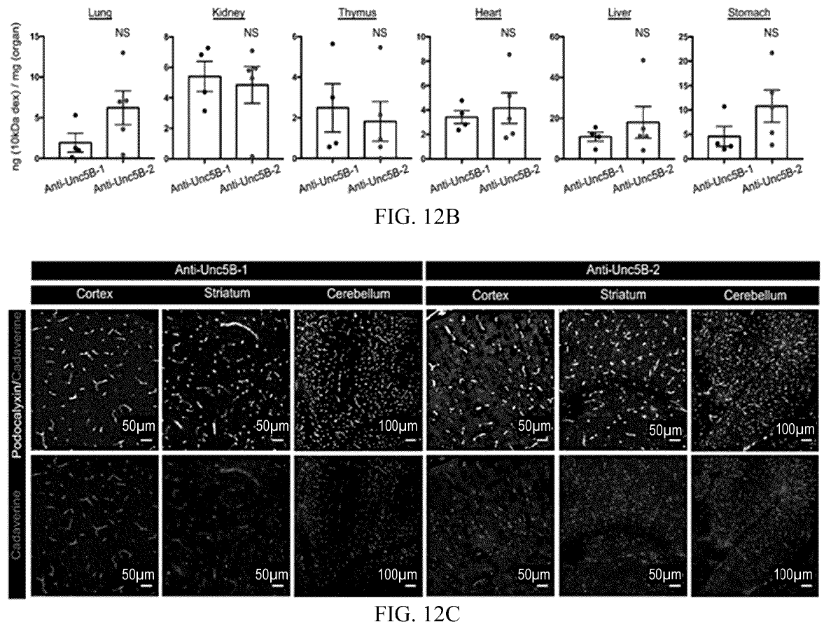

Mechanisms driving this effect were then evaluated. Because Claudin5 is an essential TJ component that regulates paracellular BBB properties, Unc5B might regulate BBB integrity at least in part by maintaining Claudin5 expression. Although Claudin5 is much more abundant than Unc5B18, in certain embodiments Unc5B may regulate Claudin5 expression levels. In one instance, Claudin5 levels are reduced in both the endothelial inducible Unc5B mice (FIG. 5E) and in embryonic brains of global Unc5B mutants8 (FIGS. 12A-12D). In another instance, siRNA knockdown of Unc5B in cultured mouse brain endothelial cells reduced Claudin5 expression (FIG. 12E). In all of these loss-of-function approaches, about a 40% reduction of Claudin5 levels was observed when Unc5B was absent. In yet another instance, overexpression of Unc5B increased Claudin5 levels (FIG. 12F).

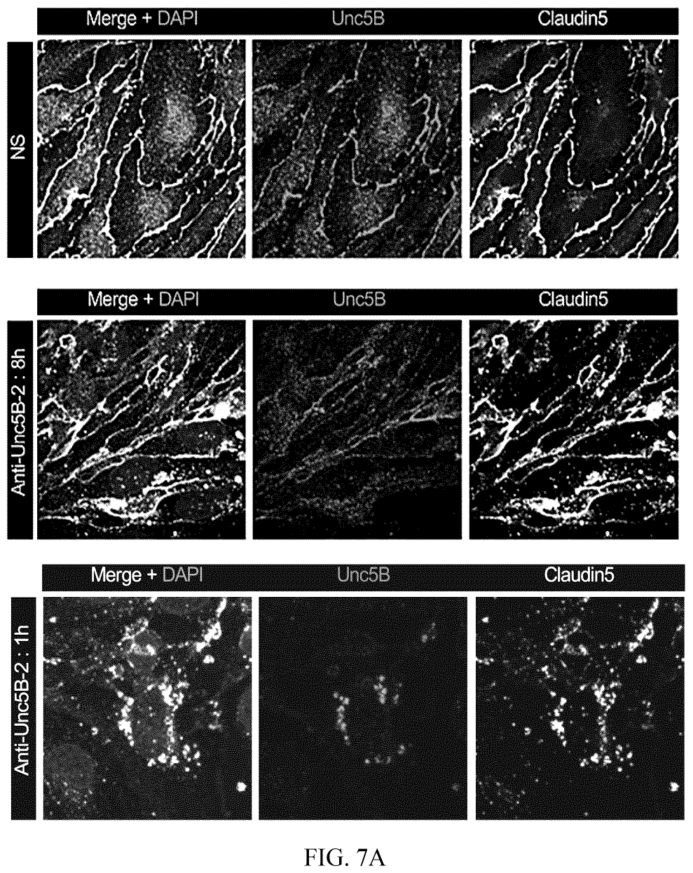

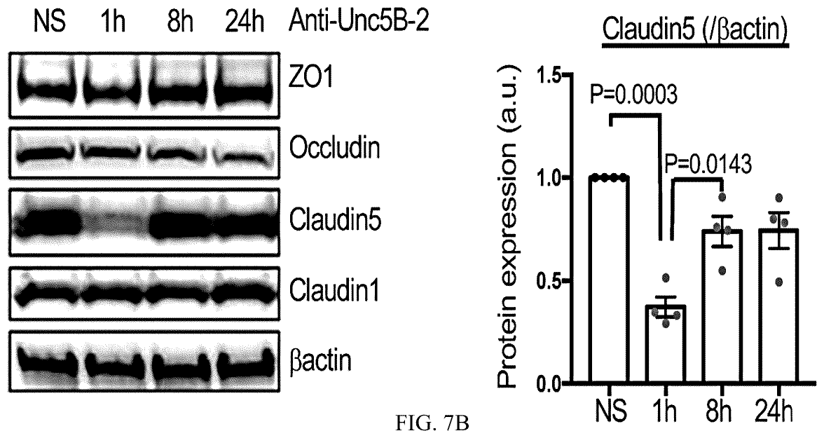

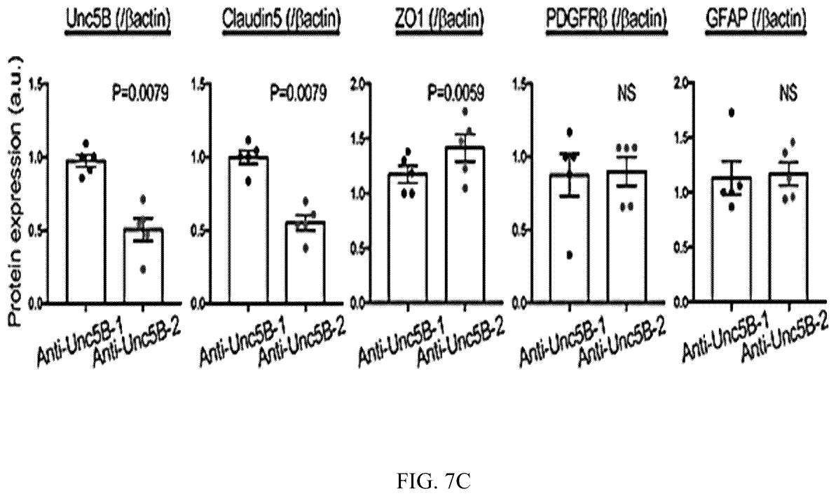

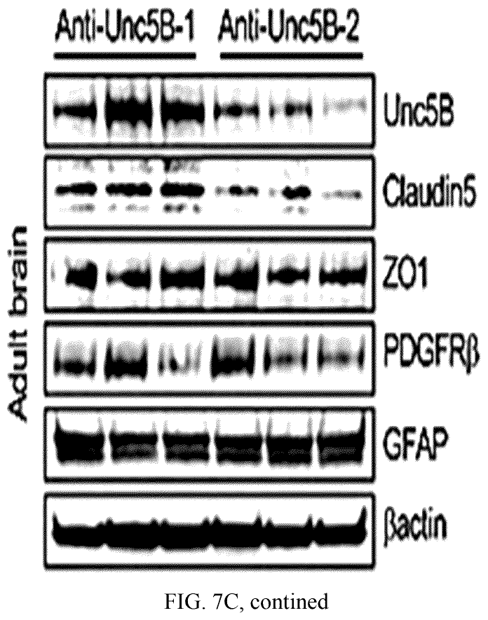

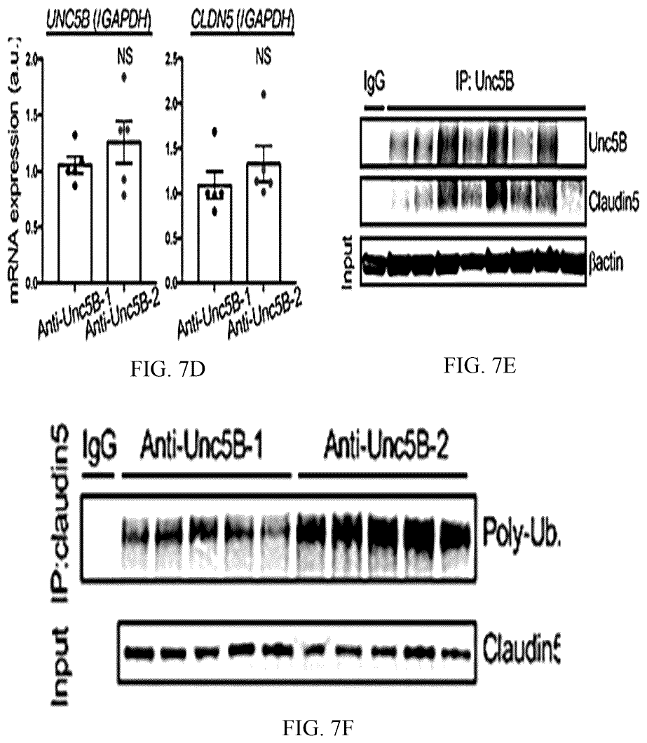

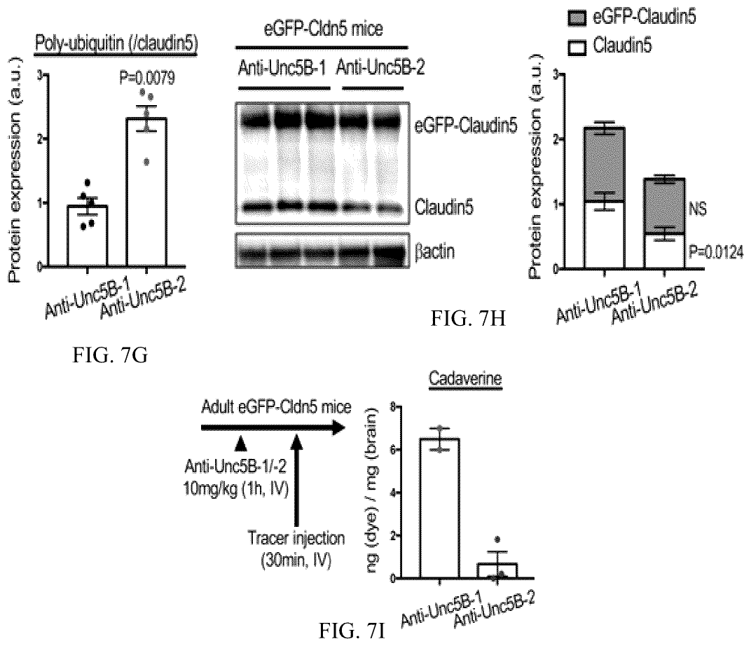

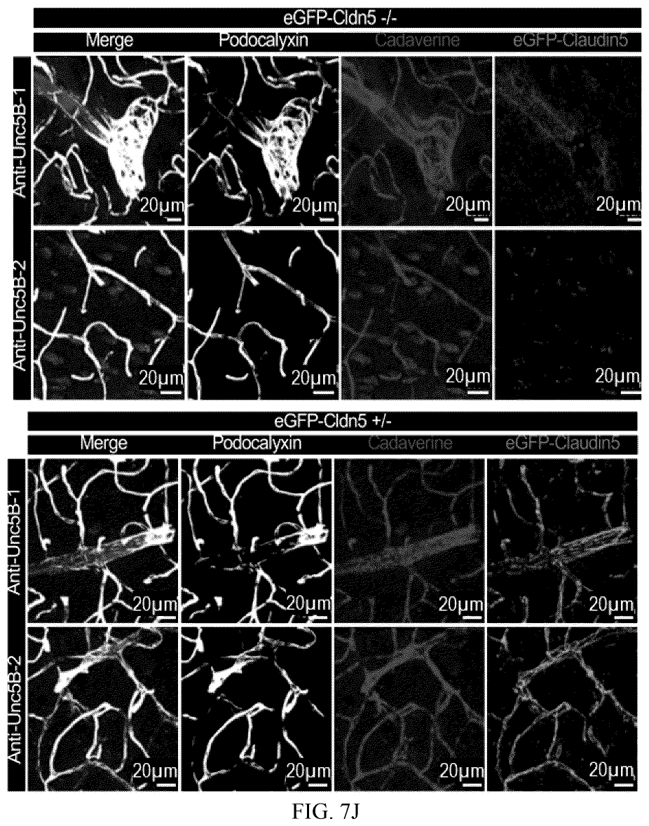

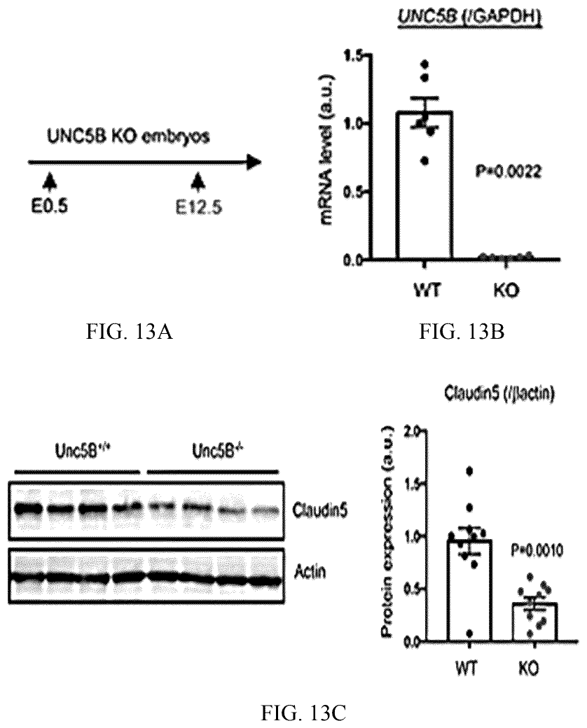

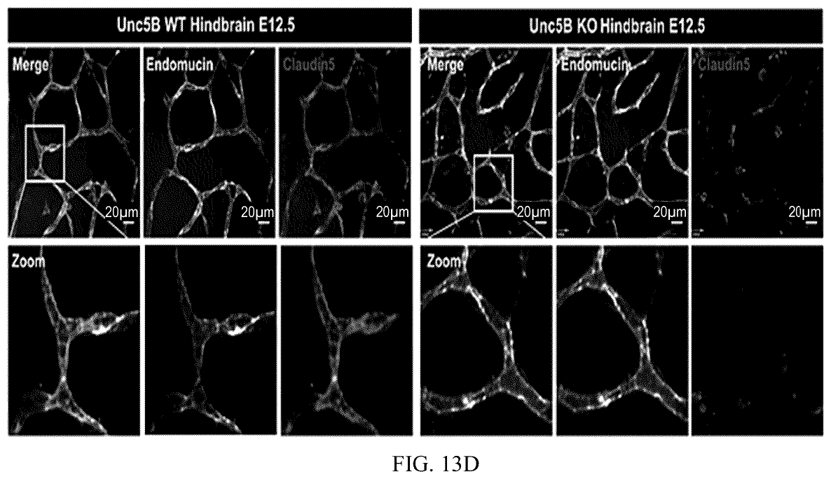

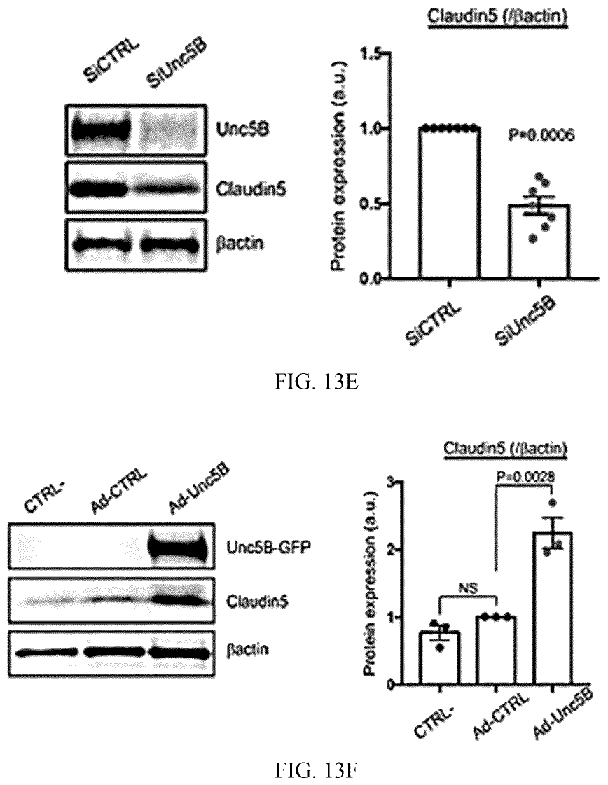

To probe how Unc5B regulated Claudin5, cultured mouse brain endothelial cells were treated with anti-Unc5B-2, which led to transient Unc5B internalization after 1 hour, followed by its return to the cell membrane 8 hours later (FIG. 7A). Claudin5 was internalized along with Unc5B, and also returned to the surface along with Unc5B after 8 hrs (FIG. 7A). Claudin5 internalization was accompanied by a transient reduction in Claudin5 protein levels in anti-Unc5B-2 treated cells, suggesting that some of the internalized molecules could be degraded (FIG. 7B). Unc5B and Claudin5 protein levels in both adult and neonatal brain lysates were also decreased 1h after anti-Unc5B-2 treatment, while levels of PDGFRb. GFAP and ZO1 were similar to controls treated with anti-Unc5B-1 (FIGS. 7C and 13). mRNA levels of Claudin5 and Unc5B remained unchanged by antibody treatment (FIG. 7D), suggesting that Unc5B regulated Claudin5 protein levels. Immunoprecipitation of Unc5B and Western blotting for Claudin5 showed that Unc5B co-immunoprecipitated with Claudin5 in WT brain protein lysate (FIG. 7E), consistent with a protein-protein interaction. Immunoprecipitation of Claudin5 and Western blotting for poly-Ubiquitin showed enhanced ubiquitination of Claudin5 in anti-Unc5B-treated brain lysates when compared to anti-Unc5B-1 treated lysates (FIGS. 7F-7G). These findings suggest that Unc5B stabilized Claudin5 at TJs by a potential physical interaction, thereby protecting it from proteasomal degradation.

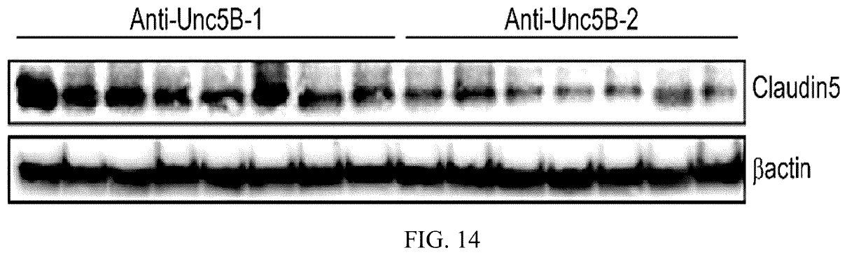

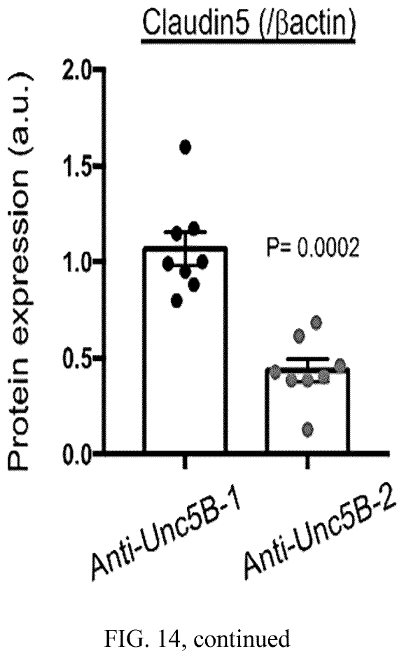

If Claudin5 levels were functionally relevant to Unc5B action at the BBB, raising Claudin5 levels should protect mice from anti-Unc5B-2 mediated BBB leakage. To test this hypothesis, eGFP::Claudin5 transgenic mice were used that express 2-fold higher Claudin5 levels compared to wildtype littermates (FIGS. 14A. 14B). Anti-Unc5B-2 treatment decreased levels of endogenous Claudin5 but did not affect significantly the eGFP::Claudin5 transgene levels. Overall, anti-Unc5B-2 treatment reduced Claudin5 expression to WT levels (FIG. 7H). Injection of anti-Unc5B-2 into these mice prevented Cadaverine leak (FIGS. 7I-7J), demonstrating functional involvement of Claudin5 in Unc5B actions at the BBB.

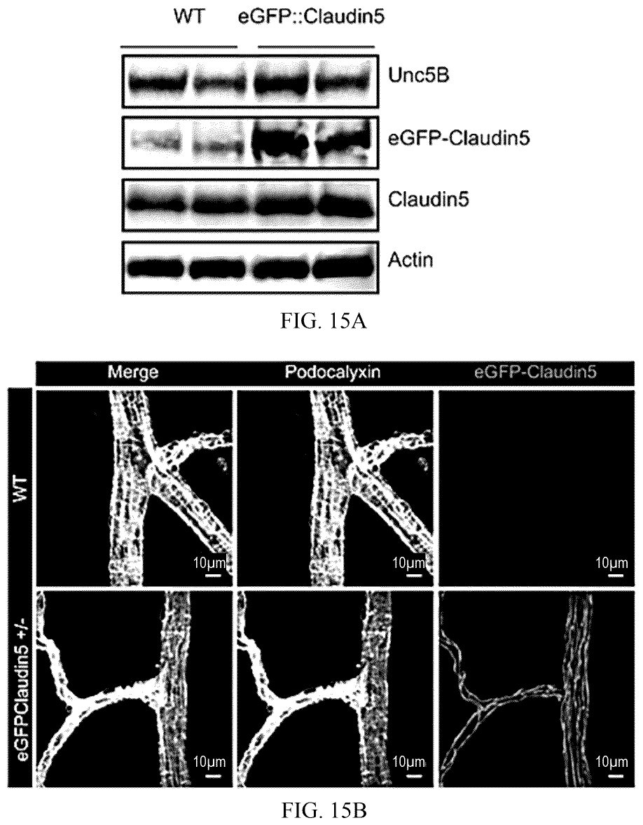

Because tracer leak in Claudin5−/− mice was restricted to molecules of less than 1 kDa 16, but Unc5B loss of function induced leakage of larger tracers up to 40 kDa. Unc5B can in one aspect affect additional BBB pathways. Unc5B inhibits Vegfr2-mediated 30) permeability signaling in cultured endothelial cells by selectively reducing phosphorylation of the Y949 residue (Y951 in humans), which activates VE-Cadherin phosphorylation and triggers disassembly of adherent junctions, in turn followed by Claudin5 downregulation. Increased Vegfr2-Y949 permeability signaling in the absence of Unc5B can in one aspect contribute to BBB opening. Western blotting of Unc5BiECko brain lysates indeed revealed increased Vegfr2-Y949 phosphorylation compared to Cre-negative littermate controls (FIG. 8A). Vegfr2-Y1173 phosphorylation, which is critical for Vegf-induced proliferation, was unaffected by loss of Unc5B function, confirming the previous in vitro results (FIG. 8A). To test the function of Vegfr2-Y949 signaling in Unc5B regulation of BBB permeability. Vegfr2-Y949-F mutant mice were used, which carry an inactivating substitution of tyrosine to phenylalanine and are resistant to Vegf-induced permeability. Western-blot comparison of Y949 wildtype and Y949F mutant mouse brains did not reveal any change in Claudin5 expression levels (FIG. 15A), and injection of anti-Unc5B-2 into Y949F mice still opened the BBB to Cadaverine, leading to significant dye leakage into the brain (FIGS. 8B, 8C, and 15B). These data show that Vegfr2 signaling is not critical to Unc5B effects at the BBB under homeostatic conditions.

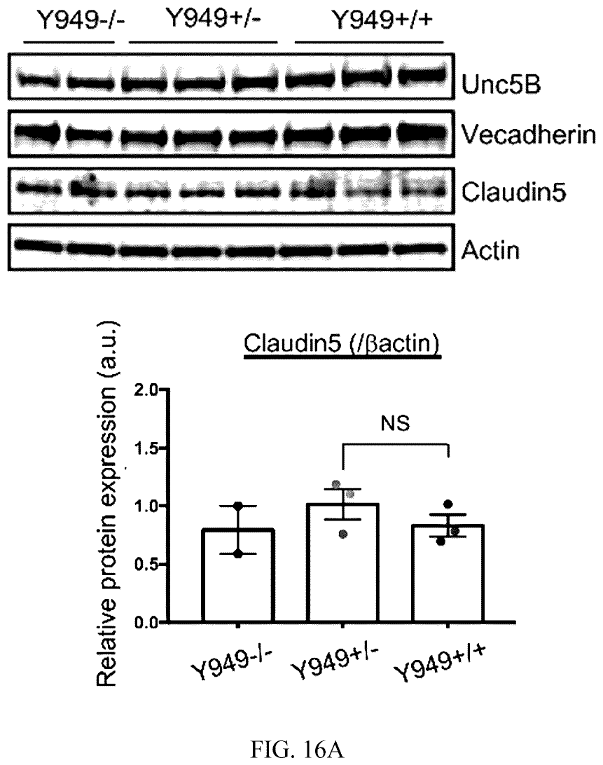

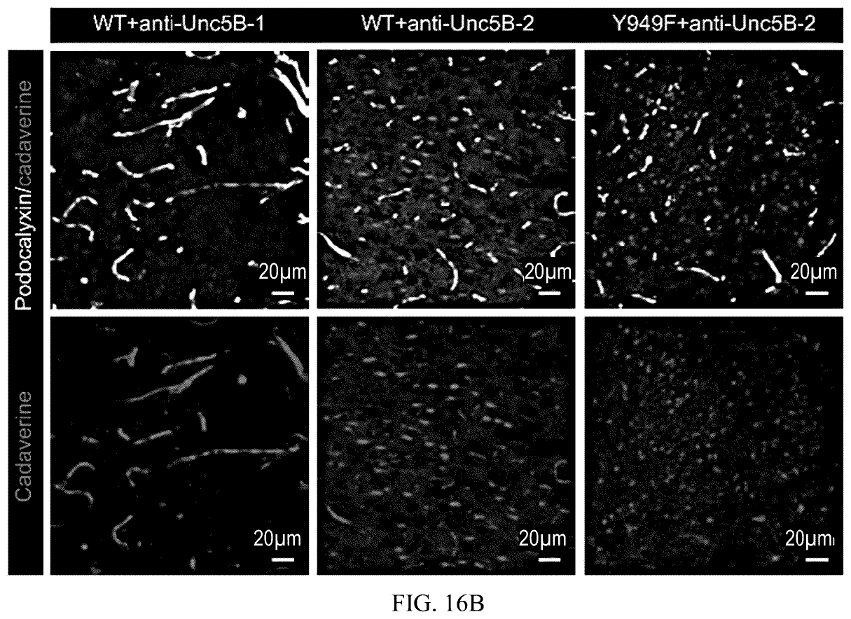

Next, studies were conducted to investigate if Unc5B inhibition affected mRNA levels for BBB components. While anti-Unc5B-1- or -2 treated mice showed similar levels of Claudin5 mRNA (FIG. 7D), qPCR analysis of Unc5BiECko mice revealed a 50% downregulation of Claudin5 mRNA compared to Cre negative littermate controls (FIG. 16A). Further analysis demonstrated that mRNAs for Wnt/b-catenin target genes Slc2a1 (Glut1) was downregulated, while PVLAP, which is decreased by Wnt/b-catenin signaling, was increased in Unc5BiECko mice (FIG. 16A). This suggested that Unc5B affected Wnt/b-catenin signaling, the major known pathway inducing BBB formation and maintenance. Western blotting for various Wnt/b-catenin signaling components such as b-catenin, LEF1 and pLRP6 revealed significant downregulation of all these components in brain lysates from Unc5BiECko mice (FIGS. 8D-8E). Immunostaining of Unc5BiECko mice brains showed a strong decrease in endothelial LEF1 expression upon Unc5B deletion (FIGS. 8F-8G). Similar results were obtained in cultured brain endothelial cells after siRNA mediated Unc5B knockdown where LEF1 was decreased and PLVAP was increased (FIGS. 16B-16C). To test whether reduced Wnt/b-catenin signaling functionally contributed to BBB opening in the absence of Unc5B. Unc5BiECko were crossed with mice overexpressing an activated form of bcatenin, which enhanced Wnt/b-catenin signaling. The resulting offspring (Unc5BiECko-Ctnnb1flex/3) showed an increased Claudin5 expression compared to Unc5BiECko mice (FIG. 8H) and did not show any leakage of cadaverine (FIG. 8I).

In summary, these data reveal Unc5B as a novel and essential regulator of BBB development and maintenance. Without wishing to be limited by any theory. Unc5B maintains Claudin5 expression at TJs by physical interactions and by promoting Wnt/b-catenin signaling to regulate expression of its target genes including Claudin5 (FIG. 8J). As shown herein, antibody-mediated Unc5B blockade can be used for transient, on-demand and reversible BBB opening in mice, and has therapeutic utility for treatment of human pathologies affecting the CNS.

### Example 3: Development of Additional Unc5B Antibodies

A Phage-Fab (antigen-binding fragment) selection was then performed using a naïve Fab library (libF, Yale University) on an immobilized recombinant rat Unc5B-ECD Fc fusion protein (R&D systems). Phage display allows for the rapid selection of highly specific antibodies from libraries of fully humanized synthetic antibody fragments (Fabs) containing billions of unique clones. During selection of Fabs, phage particles fused with Fabs to viral coat proteins are incubated with an unrelated protein (e.g. streptavidin) immobilized on a solid surface and allowed to bind in a step termed counterselection. Non-specific Fabs binding unrelated protein in this step remain while the vast majority of Fabs are removed and incubated with immobilized target antigen. After incubation, unbound particles are washed away and bound particles are eluted and infected into bacteria for overnight amplification. After 3-5 rounds of this process whereby libraries are enriched for Fabs able to bind target, individual clones are grown in 96-well format and tested by ELISA for their ability to bind antigen specifically.

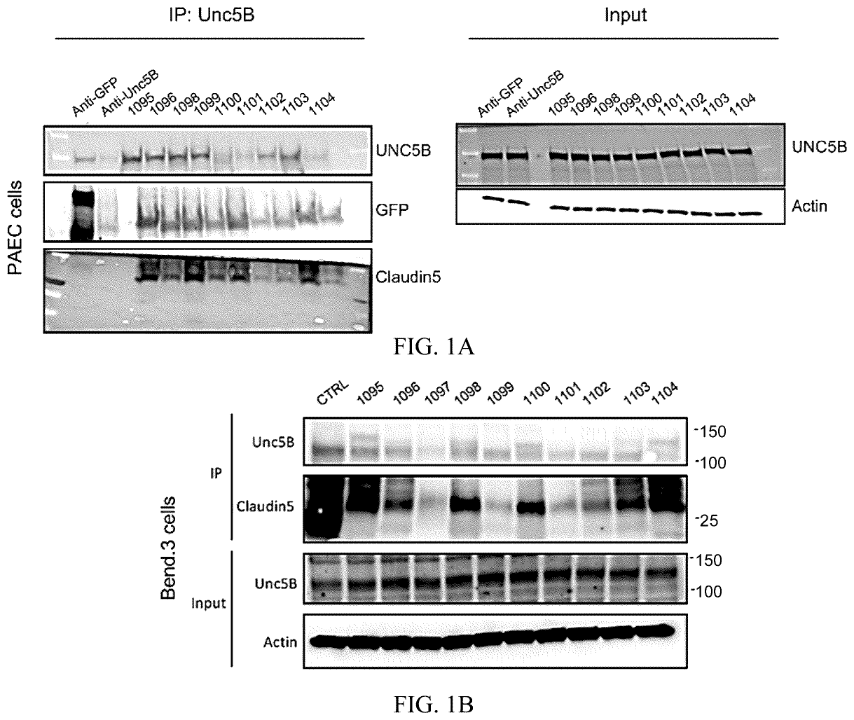

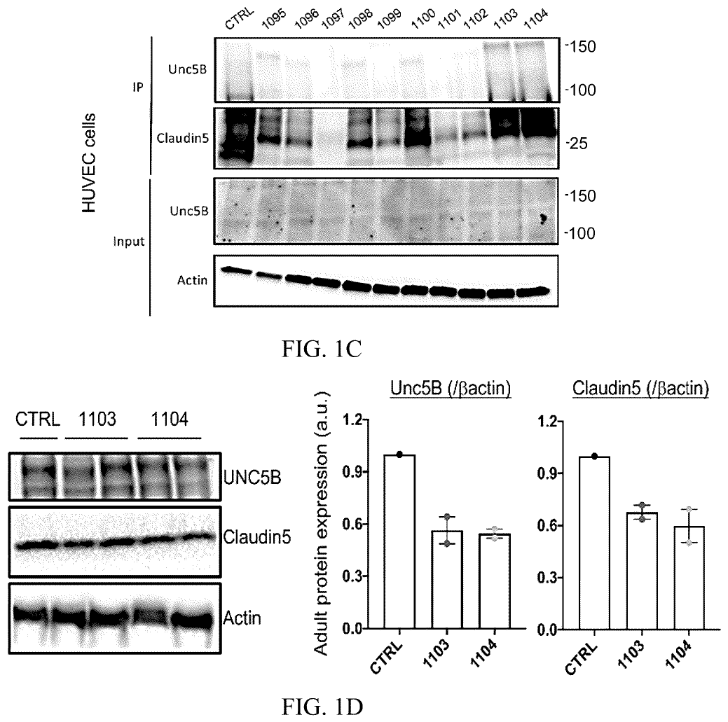

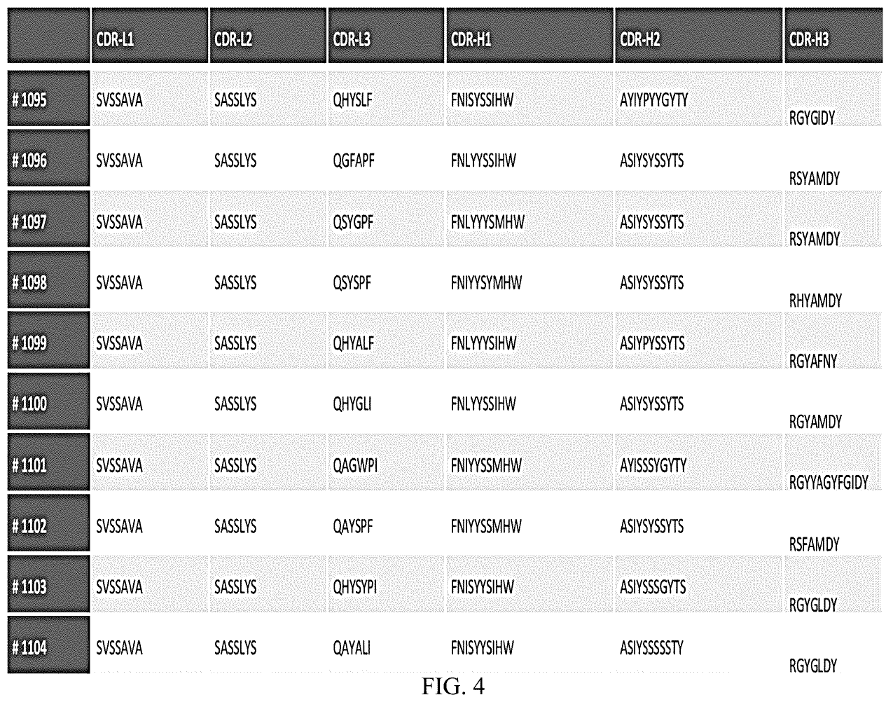

Ten unique and different positive Fab were selected over 5 rounds of selection (name hereafter 1095 to 1104) (FIGS. 3-4), which were subcloned before antibody production (Proteogenix company, France). Light (LC) and Heavy (HC) chains were sequenced for each antibody (FIGS. 1A-ID and 2A-2D). These antibodies were then tested to their abilities to recognize rat Unc5B using Porcine aortic endothelial cells (PAECs) transfected with rat Unc5B coupled to GFP. 1 h after antibody treatment, Unc5B immunoprecipitation revealed that all antibodies could recognize rat-Unc5B as well as Claudin5 (FIG. 1A) indicating physical interaction between the two proteins. Using the same methodology, these antibodies were then observed whether they could also recognize mouse Unc5B using immortalized mouse brain Bend.3 cells as well as human Unc5B using human umbilical vein HUVEC cells (FIG. 1A-1C).

Expression of Unc5B and Claudin5 upon antibody treatment was assessed in Bend.3 cells and both Unc5B and Claudin5 were found to be downregulated about 40% 1h after antibody treatment (FIG. 1D). BBB permeability was assessed in vitro by measurement of real-time transendothelial electrical resistance (TEER) using ECIS® device after control or antibody treatment in HUVEC and Bend.3 cells (FIGS. 2A-2D). Several positive controls were used (83.4 for HUVEC and 83.21 for both HUVEC and Bend.3 cells). Several antibodies were found which were able to induce permeability in both cells line including 1095, 1102, 1103 and 1104 (FIG. 2A). Interestingly, 1097 treatment induced a junction tightening as TEER measurement indicated as decreased basal permeability (FIG. 2B).

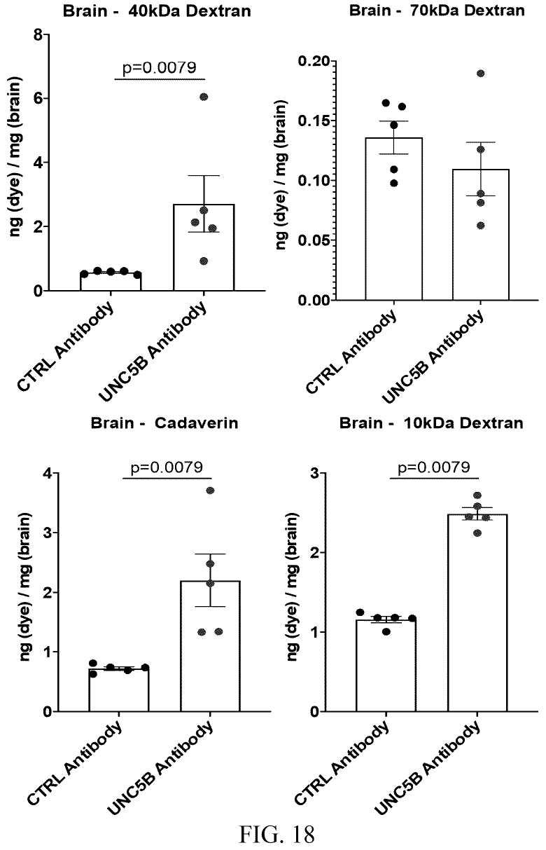

To test if antibodies opened the BBB in vivo, adult mice were injected intravenously (iv) with 1103 and 1104 antibodies (10 mg/kg, 1h) followed by iv injection of tracers of various molecular weights for 30 min before sacrifice and analysis. An increased leak of Cadaverine (MW 950 Da) was observed upon 1103 and 1104 treatment in the adult brain (FIG. 2C). The size-selectivity window seen in Unc5BiECko mice being compatible with delivery of small molecule up to 40 kDa, cadaverine and nanobodies were co-injected 1h after 1103 injection (iv, 10 mg/kg). A large cadaverine leak was observed in the mouse brain which as accompanied with nanobody extravasation (FIG. 2D). Having observed the ability of 1103 and 1104 antibodies to open up permeability in the BBB, follow-up studies were conducted to determine the maximum size of molecules that would be allowed through the opening. Similar animal studies using various sizes of dextran tracers ranging from 10-70 kDa found that tracers up to 40 kDa were able to diffuse into brain tissue, but not the 70 kDa tracer (FIG. 18).

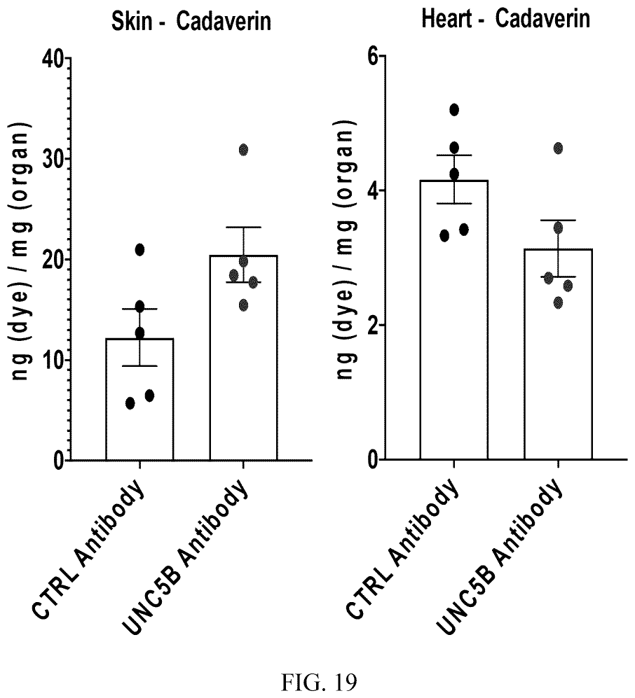

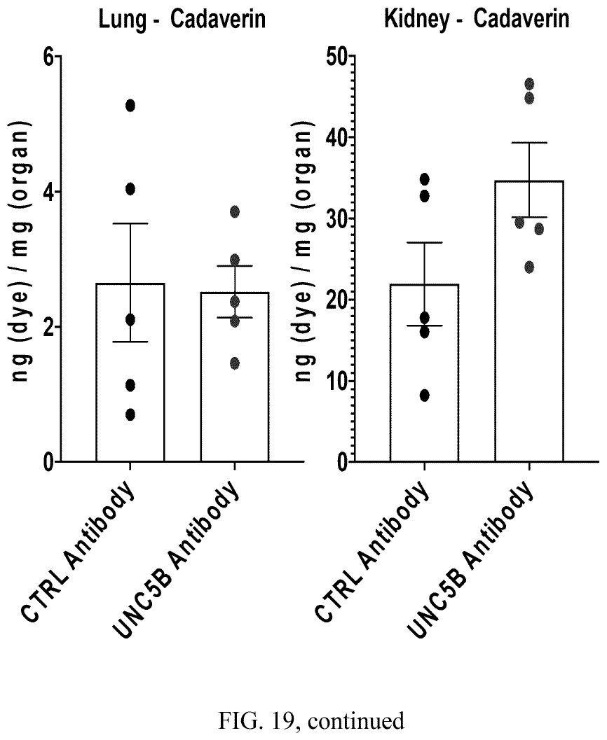

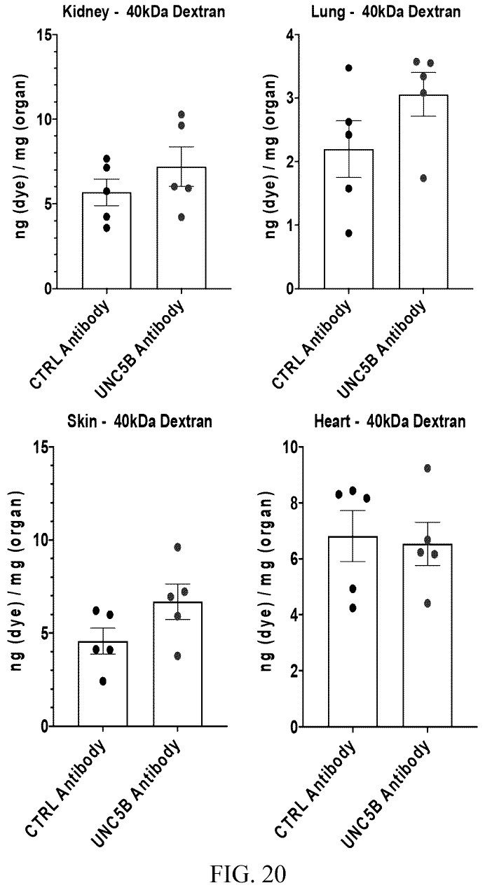

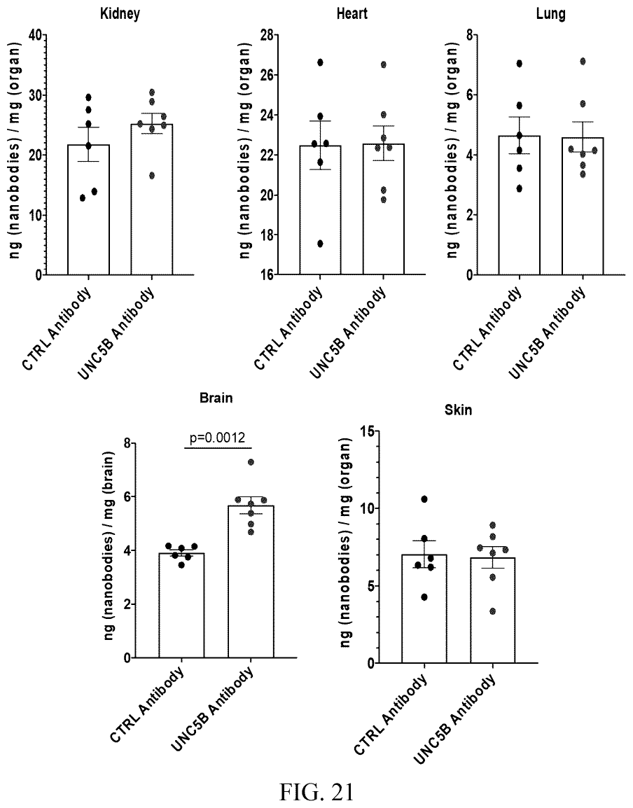

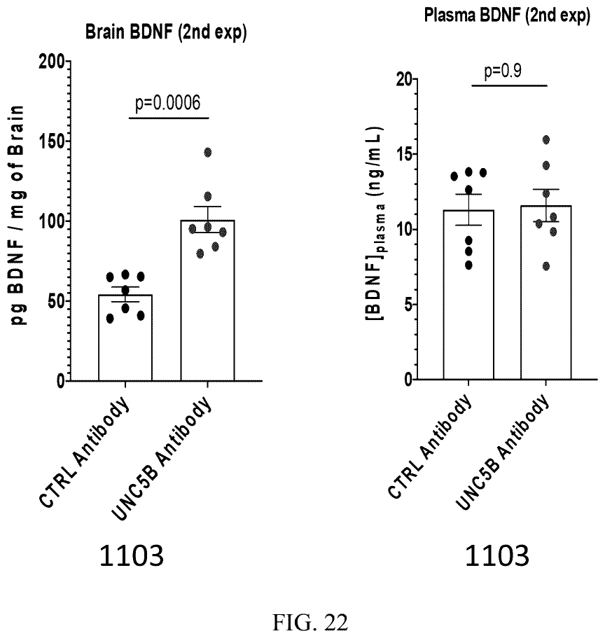

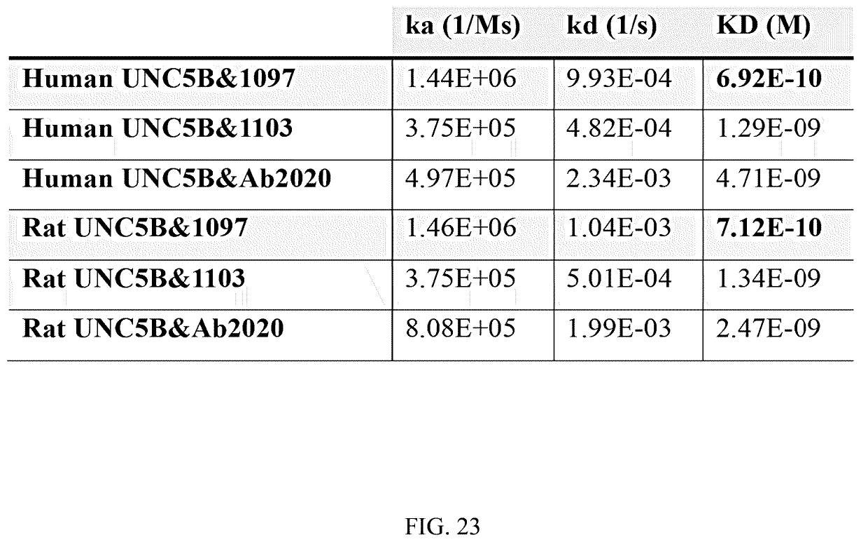

The ability of treatment with anti-Unc5B antibody 1103 to induce permeability in other organ tissue was then assessed. Studies were conducted using cadaverine (FIG. 19), dextran (FIG. 20), and nanobodies (FIG. 21) in combination with 1103 antibody. In each case, treatment with 1103 antibody induced significant permeability only in brain tissue, but not in skin, heart, kidney, and lung. Lastly, the use of anti-Unc5B treatment to open the BBB could be used to deliver growth factors to CNS tissue for the treatment of various neurological diseases. To see if 1103 treatment could allow the transport of an exogenously administered growth factor into CNS tissue, 1103 antibody treatment was followed by the injection of brain-derived neurotrophic factor (BDNF) (FIG. 22). As compared to control, 1103 enabled significant transport of detectable BDNF into brain tissue. Biacore analysis of binding affinity for Abs 1103 and 1097 reveal a KD of 1.3 nM (1103) and 700 pM (1097) (FIG. 23).

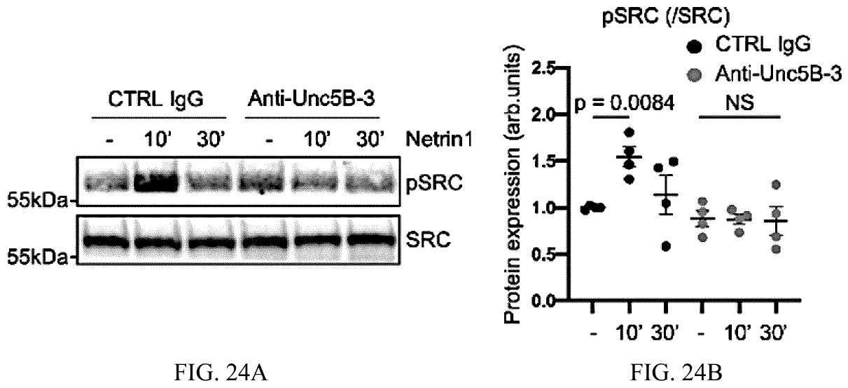

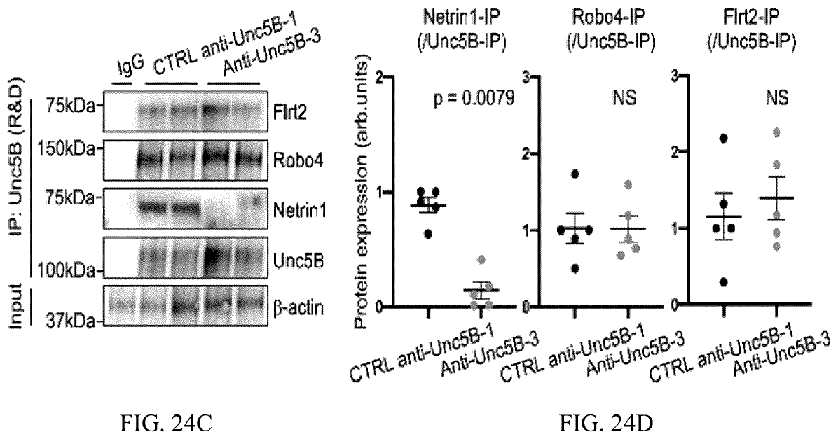

Studies were then conducted to assess the effect of 1103 binding on Netrin-1 binding and signaling function, which found that 1103 blocked Netrin-1-induced Src phosphorylation in brain ECs in vitro (FIGS. 24A-24B). To test effects on ligand binding in vivo, a control antibody or 1103 was injected i.v (10 mg/kg for 1 h), followed by Unc5B immunoprecipitation from brain lysates using a commercial antibody recognizing the Unc5B ECD. Western blotting revealed that 1103 blocked Netrin-1 binding to Unc5B in vivo, while Robo4 and Flrt2 could still interact with Unc5B (FIGS. 24C-24D).

Altogether, novel antibodies were generated, all of which target an endothelial receptor (Unc5B) critically involved in BBB homeostasis. These antibodies can be used to open the BBB to several tracers and are compatible with delivery of small molecules and single-domain antibody therapeutics such as nanobodies.

### Enumerated Embodiments

The following enumerated embodiments are provided, the numbering of which is not to be construed as designating levels of importance.
Embodiment 1 provides an isolated binding polypeptide comprising an antigen-binding domain that specifically binds to an epitope of human, mouse, and/or rat Unc5B.
Embodiment 2 provides the binding polypeptide of embodiment 1, wherein the antigen-binding domain comprises:
A heavy chain variable region that comprises three heavy chain complementarity determining regions (HCDRs), wherein HCDR1 comprises an amino acid sequence selected from the group comprising SEQ ID NOs: 13-19, HCDR2 comprises an amino acid sequence selected from the group comprising SEQ ID NOs: 20-24, and HCDR3 comprises an amino acid sequence selected from the group comprising SEQ ID NOs: 25-32; and A light chain variable region that comprises three light chain complementarity determining regions (LCDRs), wherein LCDR1 comprises the amino acid sequence SVSSAVA (SEQ ID NO. 1), LCDR2 comprises the amino acid sequence SASSLYS (SEQ ID NO. 2), and LCDR3 comprises and amino acid sequence selected from the group comprising SEQ ID NOs: 3-12.
Embodiment 3 provides the binding polypeptide of any one of embodiments 1-2, wherein the binding polypeptide binds an Uncoordinated 5B (Unc5B) protein.
Embodiment 4 provides the binding polypeptide of any one of embodiments 1-3, wherein the binding polypeptide comprises an antibody or an antigen-binding fragment thereof.
Embodiment 5 provides the binding polypeptide of embodiment 4, wherein the antigen-binding fragment is selected from the group consisting of a Fab, a single-chain variable fragment (scFv), and a single-domain antibody.
Embodiment 6 provides the binding polypeptide of any one of embodiments 4-5, wherein the antibody is a full-length antibody.
Embodiment 7 provides the binding polypeptide of any one of embodiments 4-6, wherein the antibody or antigen-binding fragment is a humanized antibody or an antigen-binding fragment thereof.
Embodiment 8 provides the binding polypeptide of any one of embodiments 1-7, wherein the binding polypeptide comprises a heavy chain variable region comprising an amino acid sequence having at least 80%, 85%, 90%, 95%, 96%, 96%, 97%, 98%, or 99% identity to an amino acid sequence of the heavy chain variable region selected from the group comprising SEQ ID NOs: 43-52.
Embodiment 9 provides the binding polypeptide of any one of embodiments 1-8, wherein the binding polypeptide comprises a heavy chain variable region comprising an amino acid sequence selected from the group comprising SEQ ID NOs: 43-52.
Embodiment 10 provides the binding polypeptide of any one of embodiments 1-9, wherein the binding polypeptide consists of a heavy chain variable region consisting of an amino acid sequence selected from the group comprising SEQ ID NOs: 43-52.
Embodiment 11 provides the binding polypeptide of any one of embodiments 1-10, wherein the binding polypeptide comprises a heavy chain variable region encoded by a nucleotide sequence selected from the group comprising SEQ ID NOs: 53-62.
Embodiment 12 provides the binding polypeptide of any one of embodiments 1-11, wherein the binding polypeptide comprises a light chain variable region comprising an amino acid sequence having at least 80%, 85%, 90%, 95%, 96%, 97%, 98%, or 99% identity to an amino acid sequence selected from the group comprising SEQ ID NOs: 33-42.
Embodiment 13 provides the binding polypeptide of any one of embodiments 1-12, wherein the binding polypeptide comprises a light chain variable region comprising an amino acid sequence selected from the group comprising SEQ ID NOs: 33-42.
Embodiment 14 provides the binding polypeptide of any one of embodiments 1-13, wherein the binding polypeptide consists of a light chain variable region comprising an amino acid sequence selected from the group comprising SEQ ID NOs: 33-42.
Embodiment 15 provides the binding polypeptide of any one of embodiments 1-14, wherein the binding polypeptide comprises a light chain variable region encoded by a nucleotide sequence selected from the group comprising SEQ ID NOs: 63-72.
Embodiment 16 provides a pharmaceutical composition comprising the isolated binding polypeptide of any of embodiments 1-15.
Embodiment 17 provides an isolated nucleic acid encoding a binding polypeptide comprising an antigen-binding domain that specifically binds an epitope of human, mouse, and/or rat Unc5b.
Embodiment 18 provides the nucleic acid of embodiment 17, wherein the antigen-binding domain comprises: A heavy chain variable region that comprises three heavy chain complementarity determining regions (HCDRs), wherein HCDR1 comprises an amino acid sequence selected from the group comprising SEQ ID NOs: 13-19, HCDR2 comprises and amino acid sequence selected from the group comprising SEQ ID NOs: 20-24, and HCDR3 comprises an amino acid sequence selected from the group comprising SEQ ID NOs: 25-32; and A light chain variable region that comprises three light chain complementarity determining regions (LCDRs), wherein LCDR1 comprises the amino acid sequence SVSSAVA (SEQ ID NO. 1), LCDR2 comprises the amino acid sequence SASSLYS (SEQ ID NO. 2), and LCDR3 comprises and amino acid sequence selected from the group comprising SEQ ID NOs: 3-12.
Embodiment 19 provides the nucleic acid of any one of embodiments 17-18, wherein the binding polypeptide binds a human, mouse, and/or rat Uncoordinated 5 B protein (Unc5B).
Embodiment 20 provides the nucleic acid of any one of embodiments 17-19, wherein the binding polypeptide comprises an antibody or an antigen-binding fragment thereof. Embodiment 21 provides the nucleic acid of embodiment 20, wherein the antigen-binding fragment is selected from the group consisting of a Fab, a single-chain variable fragment (scFv), and a single-domain antibody.
Embodiment 22 provides the nucleic acid of any one of embodiments 20-21, wherein the antibody is a full-length antibody.
Embodiment 23 provides the nucleic acid of any one of embodiments 20-22, wherein the antibody or antigen-binding fragment is a humanized antibody or an antigen-binding fragment thereof.
Embodiment 24 provides the nucleic acid of any one of embodiments 17-23, wherein the binding polypeptide comprises a heavy chain variable region comprising an amino acid sequence having at least 80%, 85%, 90%, 95%, 96%, 96%, 97%, 98%, or 99% identity to an amino acid sequence of the heavy chain variable region selected from the group comprising SEQ ID NOs: 43-52.
Embodiment 25 provides the nucleic acid of any one of embodiments 17-24, wherein the binding polypeptide comprises a heavy chain variable region comprising an amino acid sequence selected from the group comprising SEQ ID NOs: 43-52.
Embodiment 26 provides the nucleic acid of any one of embodiments 17-25, wherein the binding polypeptide consists of a heavy chain variable region consisting of an amino acid sequence selected from the group comprising SEQ ID NOs: 43-52.
Embodiment 27 provides the nucleic acid of any one of embodiments 17-26, wherein the binding polypeptide comprises a heavy chain variable region encoded by a nucleotide sequence selected from the group comprising SEQ ID NOs: 53-62.
Embodiment 28 provides the nucleic acid of any one of embodiments 17-27, wherein the binding polypeptide comprises a light chain variable region comprising an amino acid sequence having at least 80%, 85%, 90%, 95%, 96%, 97%, 98%, or 99% identity to an amino acid sequence selected from the group comprising SEQ ID NOs: 33-42.
Embodiment 29 provides the nucleic acid of embodiments 17-28, wherein the binding polypeptide comprises a light chain variable region comprising an amino acid sequence selected from the group comprising SEQ ID NOs: 33-42.
Embodiment 30 provides the nucleic acid of embodiments 17-29, wherein the binding polypeptide consists of a light chain variable region consisting of an amino acid sequence selected from the group comprising SEQ ID NOs: 33-42.
Embodiment 31 provides the nucleic acid of embodiment 17-30, wherein the binding polypeptide comprises a light chain variable region encoded by a nucleotide sequence selected from the group comprising SEQ ID NOs: 63-72.
Embodiment 32 provides a vector comprising the isolated nucleic acid of any one of embodiments 17-31.
Embodiment 33 provides the vector of embodiment 32, wherein the vector is an expression vector.
Embodiment 34 provides the vector of any one of embodiments 32-33, wherein the vector is selected from the group consisting of a DNA vector, an RNA vector, a plasmid, a lentiviral vector, an adenoviral vector, an adeno-associated viral vector, and a retroviral vector.
Embodiment 35 provides a host cell comprising the vector of any one of embodiments 32-34.
Embodiment 36 provides the host cell of embodiment 35, wherein the host cell is of eukaryotic or prokaryotic origin.
Embodiment 37 provides the host cell of any one of embodiments 35-36, wherein the host cell is of mammalian origin.
Embodiment 38 provides the host cell of any one of embodiment 35-37, wherein the host cell is of bacterial origin.

### Other Embodiments

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. An isolated binding polypeptide comprising an antigen-binding domain that specifically binds to an epitope of human, mouse, or rat Unc5B.

2. The binding polypeptide of claim 1, wherein the antigen-binding domain comprises:
a heavy chain variable region that comprises three heavy chain complementarity determining regions (HCDRs), wherein
HCDR1 comprises an amino acid sequence selected from the group comprising SEQ ID NOs: 13-19,
HCDR2 comprises an amino acid sequence selected from the group comprising SEQ ID NOs: 20-24, and
HCDR3 comprises an amino acid sequence selected from the group comprising SEQ ID NOs: 25-32; and
a light chain variable region that comprises three light chain complementarity determining regions (LCDRs), wherein
LCDR1 comprises the amino acid sequence SVSSAVA (SEQ ID NO. 1),
LCDR2 comprises the amino acid sequence SASSLYS (SEQ ID NO. 2), and
LCDR3 comprises and amino acid sequence selected from the group comprising SEQ ID NOs: 3-12.

3. The binding polypeptide of claim 1, wherein the binding polypeptide binds an Uncoordinated 5B (Unc5B) protein.

4. The binding polypeptide of claim 1, wherein the binding polypeptide comprises an antibody or an antigen-binding fragment thereof.

5. The binding polypeptide of claim 4, wherein at least one of the following applies:
the antigen-binding fragment is selected from the group consisting of a Fab, a single-chain variable fragment (scFv), and a single-domain antibody;
the antibody is a full-length antibody;
the antibody or antigen-binding fragment is a humanized antibody or an antigen-binding fragment thereof.

6-7. (canceled)

8. The binding polypeptide of claim 1, wherein at least one of the following applies:
the binding polypeptide comprises a heavy chain variable region comprising an amino acid sequence having at least 80%, 85%, 90%, 95%, 96%, 96%, 97%, 98%, or 99% identity to an amino acid sequence of the heavy chain variable region selected from the group comprising SEQ ID NOs: 43-52;
the binding polypeptide comprises a heavy chain variable region comprising an amino acid sequence selected from the group comprising SEQ ID NOs: 43-52;
the binding polypeptide consists of a heavy chain variable region consisting of an amino acid sequence selected from the group comprising SEQ ID NOs: 43-52;
the binding polypeptide comprises a heavy chain variable region encoded by a nucleotide sequence selected from the group comprising SEQ ID NOs: 53-62;
the binding polypeptide comprises a light chain variable region comprising an amino acid sequence having at least 80%, 85%, 90%, 95%, 96%, 97%, 98%, or 99% identity to an amino acid sequence selected from the group comprising SEQ ID NOs: 33-42;
the binding polypeptide comprises a light chain variable region comprising an amino acid sequence selected from the group comprising SEQ ID NOs: 33-42;
the binding polypeptide consists of a light chain variable region comprising an amino acid sequence selected from the group comprising SEQ ID NOs: 33-42;
wherein the binding polypeptide comprises a light chain variable region encoded by a nucleotide sequence selected from the group comprising SEQ ID NOs: 63-72.

9-15. (canceled)

16. A pharmaceutical composition comprising the isolated binding polypeptide of claim 1.

17. An isolated nucleic acid encoding a binding polypeptide comprising an antigen-binding domain that specifically binds an epitope of human, mouse, or rat Unc5b.

18. The nucleic acid of claim 17, wherein the antigen-binding domain comprises:
a heavy chain variable region that comprises three heavy chain complementarity determining regions (HCDRs), wherein
HCDR1 comprises an amino acid sequence selected from the group comprising SEQ ID NOs: 13-19,
HCDR2 comprises and amino acid sequence selected from the group comprising SEQ ID NOs: 20-24, and
HCDR3 comprises an amino acid sequence selected from the group comprising SEQ ID NOs: 25-32; and
a light chain variable region that comprises three light chain complementarity determining regions (LCDRs), wherein
LCDR1 comprises the amino acid sequence SVSSAVA (SEQ ID NO. 1),
LCDR2 comprises the amino acid sequence SASSLYS (SEQ ID NO. 2), and
LCDR3 comprises and amino acid sequence selected from the group comprising SEQ ID NOs: 3-12.

19. The nucleic acid of claim 17, wherein the binding polypeptide binds a human, mouse, and/or rat Uncoordinated 5 B protein (Unc5B).

20. The nucleic acid of claim 17, wherein the binding polypeptide comprises an antibody or an antigen-binding fragment thereof.

21. The nucleic acid of claim 20, wherein at least one of the following applies:
the antigen-binding fragment is selected from the group consisting of a Fab, a single-chain variable fragment (scFv), and a single-domain antibody;
the antibody is a full-length antibody;
the antibody or antigen-binding fragment is a humanized antibody or an antigen-binding fragment thereof.

22-23. (canceled)

24. The nucleic acid of any one of claim 17, wherein at least one of the following applies:
the binding polypeptide comprises a heavy chain variable region comprising an amino acid sequence having at least 80%, 85%, 90%, 95%, 96%, 96%, 97%, 98%, or 99% identity to an amino acid sequence of the heavy chain variable region selected from the group comprising SEQ ID NOs: 43-52;
the binding polypeptide comprises a heavy chain variable region comprising an amino acid sequence selected from the group comprising SEQ ID NOs: 43-52;
the binding polypeptide consists of a heavy chain variable region consisting of an amino acid sequence selected from the group comprising SEQ ID NOs: 43-52;
the binding polypeptide comprises a heavy chain variable region encoded by a nucleotide sequence selected from the group comprising SEQ ID NOs: 53-62;
the binding polypeptide comprises a light chain variable region comprising an amino acid sequence having at least 80%, 85%, 90%, 95%, 96%, 97%, 98%, or 99% identity to an amino acid sequence selected from the group comprising SEQ ID NOs: 33-42;
the binding polypeptide comprises a light chain variable region comprising an amino acid sequence selected from the group comprising SEQ ID NOs: 33-42;
the binding polypeptide consists of a light chain variable region consisting of an amino acid sequence selected from the group comprising SEQ ID NOs: 33-42;
the binding polypeptide comprises a light chain variable region encoded by a nucleotide sequence selected from the group comprising SEQ ID NOs: 63-72.

25-31. (canceled)

32. A vector comprising the isolated nucleic acid of claim 17.

33. The vector of claim 32, wherein the vector is an expression vector.

34. The vector of claim 32, wherein the vector is selected from the group consisting of a DNA vector, an RNA vector, a plasmid, a lentiviral vector, an adenoviral vector, an adeno-associated viral vector, and a retroviral vector.

35. A host cell comprising the vector of claim 32.

36. The host cell of claim 35, wherein at least one of the following applies:
the host cell is of eukaryotic or prokaryotic origin;
the host cell is of mammalian origin; or
the host cell is of bacterial origin.

37-38. (canceled)
